SEQUENCE LISTING (138 sequences)

SEQ ID NO: 1 - protein, 10 aa, Artificial Sequence
  histone H3, residue 4-13
KQTARKSTGG

SEQ ID NO: 2 - protein, 17 aa, Artificial Sequence
  nuclear localization sequence
KRPAATKKAGQAKKKKL

SEQ ID NO: 3 - protein, 4 aa, Artificial Sequence
  v
GGGG

SEQ ID NO: 4 - DNA, 55 bp, Artificial Sequence
  TGF111
gctagatctgggaacctgatcatgtagatcgaatggactctaaatccgttcagcc

SEQ ID NO: 5 - DNA, 55 bp, Artificial Sequence
  TGF112
gatactagttggcggaaaccccgggaatctaacccggctgaacggatttagagtc

SEQ ID NO: 6 - DNA, 35 bp, Artificial Sequence
  TGF113
ctagatctatgactagtatccttagcgaaagctaa

SEQ ID NO: 7 - DNA, 35 bp, Artificial Sequence
  TGF114
atactagtcatagatctagcgttacaagtattaca

SEQ ID NO: 8 - DNA, 37 bp, Artificial Sequence
  TGF117
tcccatggttatgacaacttgacggctacatcattca

SEQ ID NO: 9 - DNA, 37 bp, Artificial Sequence
  TGF118
cgcatatgttaattcctcctgttagcccaaaaaacgg

SEQ ID NO: 10 - DNA, 28 bp, Artificial Sequence
  TGF119
cactgcaggaaatgaggccgctcatggc

SEQ ID NO: 11 - DNA, 29 bp, Artificial Sequence
  TGF120
cggaattcgtaacgccagggttttcccag

SEQ ID NO: 12 - DNA, 37 bp, Artificial Sequence
  TGF121
accatggtggcttactatgttggcactgatgagggtg

SEQ ID NO: 13 - DNA, 26 bp, Artificial Sequence
  TGF122
gactgcagatgcgccgcgtgcggctg

SEQ ID NO: 14 - DNA, 38 bp, Artificial Sequence
  TGF141
agtctagatgcgtttctacaaactcttttgtttatttt

SEQ ID NO: 15 - DNA, 34 bp, Artificial Sequence
  TGF142
agctgcagagtggaacgaaaactcacgttaaggg

SEQ ID NO: 16 - DNA, 49 bp, Artificial Sequence
  TGF156
  misc_feature (31)..(32): n is a, c, g, or t
gggcctggtaaaatcctatcgagttttcgmnnatagttcscaagagtcg

SEQ ID NO: 17 - DNA, 42 bp, Artificial Sequence
  TGF157
  misc_feature (27)..(28): n is a, c, g, or t
cccgagtacatcccgaacccatctgmnngaagttcaccatag

SEQ ID NO: 18 - DNA, 47 bp, Artificial Sequence
  TGF158
  misc_feature (29)..(30): n is a, c, g, or t
gcgttcaagaccaaaacctgcacctatmnntggtttgtcaatacccc

SEQ ID NO: 19 - DNA, 27 bp, Artificial Sequence
  TGF159
ataggtgcaggttttggtcttgaacgc

SEQ ID NO: 20 - DNA, 43 bp, Artificial Sequence
  TGF169-1
aaagttcagtatcattattaatacccattctctccacgtattc

SEQ ID NO: 21 - DNA, 43 bp, Artificial Sequence
  TGF169-1
aaagttcagtatcattattaataccccatctctccacgtattc

SEQ ID NO: 22 - DNA, 43 bp, Artificial Sequence
  TGF169-1
  misc_feature (28)..(28): n is a, c, g, or t
aaagttcagtatcattattaataccahntctctccacgtattc

SEQ ID NO: 23 - DNA, 43 bp, Artificial Sequence
  TGF169-1
aaagttcagtatcattattaatacctkbtctctccacgtattc

SEQ ID NO: 24 - DNA, 26 bp, Artificial Sequence
  TGF170
ggtattaataatgatactgaactttc

SEQ ID NO: 25 - DNA, 49 bp, Artificial Sequence
  TGF171-1
gtaaaatcctatcgagttttcgagcatagttcataagagtcggggcaag

SEQ ID NO: 26 - DNA, 49 bp, Artificial Sequence
  TGF171-2
gtaaaatcctatcgagttttcgagcatagttccaaagagtcggggcaag

SEQ ID NO: 27 - DNA, 49 bp, Artificial Sequence
  TGF171-3
  misc_feature (34)..(34): n is a, c, g, or t
gtaaaatcctatcgagttttcgagcatagttahnaagagtcggggcaag

SEQ ID NO: 28 - DNA, 49 bp, Artificial Sequence
  TGF171-4
gtaaaatcctatcgagttttcgagcatagtttkbaagagtcggggcaag

SEQ ID NO: 29 - DNA, 43 bp, Artificial Sequence
  TGF172-1
cgtgcattatatcaagagtatcccccatgaccatacaggaatc

SEQ ID NO: 30 - DNA, 43 bp, Artificial Sequence
  TGF172-2
cgtgcattatatcaagagtatccccccagaccatacaggaatc

SEQ ID NO: 31 - DNA, 43 bp, Artificial Sequence
  TGF172-3
  misc_feature (28)..(28): n is a, c, g, or t
cgtgcattatatcaagagtatccccahngaccatacaggaatc

SEQ ID NO: 32 - DNA, 43 bp, Artificial Sequence
  TGF172-4
cgtgcattatatcaagagtatcccctkbgaccatacaggaatc

SEQ ID NO: 33 - DNA, 25 bp, Artificial Sequence
  TGF173
ggggatactcttgatataatgcacg

SEQ ID NO: 34 - DNA, 43 bp, Artificial Sequence
  TGF174-1
gtttgtcaataccccattctctatccatagaaactggcccgac

SEQ ID NO: 35 - DNA, 43 bp, Artificial Sequence
  TGF174-1
gtttgtcaataccccattctctatcccaagaaactggcccgac

SEQ ID NO: 36 - DNA, 43 bp, Artificial Sequence
  TGF174-1
  misc_feature (28)..(28): n is a, c, g, or t
gtttgtcaataccccattctctatcahnagaaactggcccgac

SEQ ID NO: 37 - DNA, 43 bp, Artificial Sequence
  TGF174-1
gtttgtcaataccccattctctatctkbagaaactggcccgac

SEQ ID NO: 38 - DNA, 25 bp, Artificial Sequence
  TGF175
gatagagaatggggtattgacaaac

SEQ ID NO: 39 - DNA, 51 bp, Artificial Sequence
  TGF179
gtatctgcgcagtaagatgcgccccgcattgggaacctgatcatgtagatc

SEQ ID NO: 40 - DNA, 50 bp, Artificial Sequence
  TGF180
agcctgctcgttgagcaggcttttcgaatttggcggaaaccccgggaatc

SEQ ID NO: 41 - DNA, 50 bp, Artificial Sequence
  TGF181
gcattttgctattaagggattgacgagggcgtatctgcgcagtaagatgc

SEQ ID NO: 42 - DNA, 38 bp, Artificial Sequence
  TGF182
gtctcgagcatgcaaaaaagcctgctcgttgagcaggc

SEQ ID NO: 43 - DNA, 47 bp, Artificial Sequence
  TGF183
ggcctgctgactttctcgccgatcaaaaggcattttgctattaaggg

SEQ ID NO: 44 - DNA, 40 bp, Artificial Sequence
  TGF184
cagtgcacggctaactaagcggcctgctgactttctcgcc

SEQ ID NO: 45 - DNA, 30 bp, Artificial Sequence
  TGF185
cgactagtatggataaaaaaccattagatg

SEQ ID NO: 46 - DNA, 34 bp, Artificial Sequence
  TGF193
gcactagtaattcctcctgttagcccaaaaaaac

SEQ ID NO: 47 - DNA, 30 bp, Artificial Sequence
  TGF194
aagtcgaccatcatcatcatcatcattgag

SEQ ID NO: 48 - DNA, 60 bp, Artificial Sequence
  TGF255
gctcaaaagcaggtaactatataacaagactaaggcaaaccggatccccgggttaattaa

SEQ ID NO: 49 - DNA, 60 bp, Artificial Sequence
  TGF256
ccagacctgatgaaattcttgcgcataacgtcgccatctggaattcgagctcgtttaaac

SEQ ID NO: 50 - DNA, 22 bp, Artificial Sequence
  TGF257
gttactgaagagtacgtgagcg

SEQ ID NO: 51 - DNA, 22 bp, Artificial Sequence
  TGF258
atgaaagaagaacctcagtggc

SEQ ID NO: 52 - DNA, 37 bp, Artificial Sequence
  TGF281
ctcaagcttatggtatatcgtaacaggtcaaagagcg

SEQ ID NO: 53 - DNA, 51 bp, Artificial Sequence
  TGF283
tattctagagactacagtataaaaataaataaaaatgggcatcacagaaac

SEQ ID NO: 54 - DNA, 60 bp, Artificial Sequence
  TGF293
tctctagagaaaacctgtattttcagtccggcgccggcaccccggtgaccgccccgctgg

SEQ ID NO: 55 - DNA, 60 bp, Artificial Sequence
  TGF294
gtgaccgccccgctggcgggcactatctggaaggtgctggccagcgaaggccagacggtg

SEQ ID NO: 56 - DNA, 60 bp, Artificial Sequence
  TGF295
gcgaaggccagacggtggccgcaggcgaggtgctgctgattctggaagccatgaagatgg

SEQ ID NO: 57 - DNA, 59 bp, Artificial Sequence
  TGF296
ctggaagccatgaagatggaaaccgaaatccgcgccgcgcaggccgggaccgtgcgcgg

SEQ ID NO: 58 - DNA, 60 bp, Artificial Sequence
  TGF297
ggccgggaccgtgcgcggtatcgcggtgaaagccggcgacgcggtggcggtcggcgacac

SEQ ID NO: 59 - DNA, 44 bp, Artificial Sequence
  TGF298
ggagatcttcacgccagggtcatcagggtgtcgccgaccgccac

SEQ ID NO: 60 - DNA, 21 bp, Artificial Sequence
  TGF317
ggttaattcctcctgttagcc

SEQ ID NO: 61 - DNA, 21 bp, Artificial Sequence
  TGF318
gttagcaaaggtgaagaactg

SEQ ID NO: 62 - DNA, 60 bp, Artificial Sequence
  TGF319
ggctaacaggaggaattaaccatgaaaaggccggcggccacgaaaaaggccggccaggca

SEQ ID NO: 63 - DNA, 60 bp, Artificial Sequence
  TGF320
gaaaaaggccggccaggcaaaaaagaaaaagggtggcggcggtagcaagcagaccgcccg

SEQ ID NO: 64 - DNA, 53 bp, Artificial Sequence
  TGF321
cagttcttcacctttgctaacccctccggtggactaacgggcggtctgcttgc

SEQ ID NO: 65 - DNA, 53 bp, Artificial Sequence
  TGF322
cagttcttcacctttgctaacccctccggtggatttacgggcggtctgcttgc

SEQ ID NO: 66 - DNA, 45 bp, Artificial Sequence
  TGF339
ggcggtagcgttagcaaaggtgaagaactgtttaccggcgttgtg

SEQ ID NO: 67 - DNA, 44 bp, Artificial Sequence
  TGF340
tttgctaacgctaccgccgccaccctttttcttttttgcctggc

SEQ ID NO: 68 - DNA, 60 bp, Artificial Sequence
  TGF341
ttaaccatgaagcagaccgcccgttagtccaccggaggggttagcaaaggtgaagaactg

SEQ ID NO: 69 - DNA, 32 bp, Artificial Sequence
  TGF342
tctgcttcatggttaattcctcctgttagccc

SEQ ID NO: 70 - DNA, 60 bp, Artificial Sequence
  TGF343
ttaaccatgaagcagaccgcccgtaaatccaccggaggggttagcaaaggtgaagaactg

SEQ ID NO: 71 - DNA, 60 bp, Artificial Sequence
  TGF344
ttaaccatgaagcagaccgcccgttagagtaccggaggggttagcaaaggtgaagaactg

SEQ ID NO: 72 - DNA, 60 bp, Artificial Sequence
  TGF345
ttaaccatgaagcagaccgcccgttagagcaccggaggggttagcaaaggtgaagaactg

SEQ ID NO: 73 - DNA, 47 bp, Artificial Sequence
  TGF329
agaatttactatggtgaacttctttcagatgggttcgggatgtactc

SEQ ID NO: 74 - DNA, 52 bp, Artificial Sequence
  TGF346
catgctggcccccaccctgtataactacgcgcggaaactggaccggatcctg

SEQ ID NO: 75 - DNA, 49 bp, Artificial Sequence
  TGF347
gcagccgctgcccatctgaaagaagttgaccattgtaaactcttccagg

SEQ ID NO: 76 - DNA, 50 bp, Artificial Sequence
  TGF348
catgctggcccccaacctgtataactacgcgcggaaactggatcgcgctc

SEQ ID NO: 77 - DNA, 48 bp, Artificial Sequence
  TGF349
gcagccgctgcccatctgaaaaaagttcagcattgtaaactcttccag

SEQ ID NO: 78 - DNA, 20 bp, Artificial Sequence
  TGF350
ggtggcgctagccagcttgg

SEQ ID NO: 79 - DNA, 19 bp, Artificial Sequence
  TGF351
gcggccgctttaaacccgc

SEQ ID NO: 80 - DNA, 41 bp, Artificial Sequence
  TGF352
ccaagctggctagcgccaccatgaagcagaccgcccgttag

SEQ ID NO: 81 - DNA, 44 bp, Artificial Sequence
  TGF353
gcgggtttaaagcggccgcttacttcttcttctttgcctggccg

SEQ ID NO: 82 - DNA, 20 bp, Artificial Sequence
  TGF354
ggtggcaagcttccgtgcag

SEQ ID NO: 83 - DNA, 20 bp, Artificial Sequence
  TGF355
acccgctgatcagcctcgac

SEQ ID NO: 84 - DNA, 41 bp, Artificial Sequence
  TGF356
ctgcacggaagcttgccaccatgaagcagaccgcccgttag

SEQ ID NO: 85 - DNA, 45 bp, Artificial Sequence
  TGF357
gtcgaggctgatcagcgggtttacttcttcttctttgcctggccg

SEQ ID NO: 86 - DNA, 49 bp, Artificial Sequence
  TGF387
ggatttagagtccgttcggtctccctgaccaggtttccggtgtttcgtc

SEQ ID NO: 87 - DNA, 49 bp, Artificial Sequence
  TGF388
gaacggactctaaatccgttcagccgggttcgattcccggggtttccgc

SEQ ID NO: 88 - DNA, 31 bp, Artificial Sequence
  TGF379
cccgtaaaagcaccggaggggtgtccaaggg

SEQ ID NO: 89 - DNA, 32 bp, Artificial Sequence
  TGF380
ggtgcttttacgggcggtctgcttcatggtgg

SEQ ID NO: 90 - DNA, 42 bp, Artificial Sequence
  TGF389
ccgcttgagagacttactctttaaacccgctgatcagcctcg

SEQ ID NO: 91 - DNA, 37 bp, Artificial Sequence
  TGF390
agtaagtctctcaagcggtggtagtccgcctccgcca

SEQ ID NO: 92 - DNA, 37 bp, Artificial Sequence
  TGF396
gcagctaaacccgctgatcagcctcgactgtgccttc

SEQ ID NO: 93 - DNA, 37 bp, Artificial Sequence
  TGF397
cagcgggtttagctgcccttgtacagctcgtccatgc

SEQ ID NO: 94 - DNA, 29 bp, Artificial Sequence
  TGF431
ccttgctcacccctccggtgctctaacgg

SEQ ID NO: 95 - DNA, 31 bp, Artificial Sequence
  TGF432
gctgtacaagcaccatcaccatcaccatggc

SEQ ID NO: 96 - DNA, 29 bp, Artificial Sequence
  TGF433
ccttgctcacccctccggtgcttttacgg

SEQ ID NO: 97 - DNA, 29 bp, Artificial Sequence
  TGF434
caccggaggggtgagcaagggcgaggagg

SEQ ID NO: 98 - DNA, 31 bp, Artificial Sequence
  TGF435
ggtgatggtgcttgtacagctcgtccatgcc

SEQ ID NO: 99 - DNA, 19 bp, Artificial Sequence
  TGF375
ggtggcgctagccagcttg

SEQ ID NO: 100 - DNA, 19 bp, Artificial Sequence
  TGF376
atggtgagcaagggcgagg

SEQ ID NO: 101 - DNA, 35 bp, Artificial Sequence
  TGF425
cgaaagcgctcctaggccaaaaagagtgtgcaaag

SEQ ID NO: 102 - DNA, 36 bp, Artificial Sequence
  TGF426
ctaggagcgctttcgtgcaagtggaagacgactttc

SEQ ID NO: 103 - DNA, 31 bp, Artificial Sequence
  TGF427
tggaacatccgtgtccaagggcgaggaactg

SEQ ID NO: 104 - DNA, 31 bp, Artificial Sequence
  TGF428
gtttttgttcgctgcccttgtacagctcgtc

SEQ ID NO: 105 - DNA, 37 bp, Artificial Sequence
  TGF429
ccttggacacggatgttccagatgtttccaggtaact

SEQ ID NO: 106 - DNA, 49 bp, Artificial Sequence
  TGF430
caagggcagcgaacaaaaacttatttctgaagaagatctgtaactcgag

SEQ ID NO: 107 - DNA, 34 bp, Artificial Sequence
  TGF105
gaggaatcccatatggataaaaaaccattagatg

SEQ ID NO: 108 - DNA, 49 bp, Artificial Sequence
  TGF106
  misc_feature (31)..(32): n is a, c, g, or t
  misc_feature (40)..(41): n is a, c, g, or t
gggcctggtaaaatcctatcgagttttcgmnnatagttmnnaagagtcg

SEQ ID NO: 109 - DNA, 29 bp, Artificial Sequence
  TGF107
cgaaaactcgataggattttaccaggccc

SEQ ID NO: 110 - DNA, 42 bp, Artificial Sequence; identical to SEQ ID NO: 17
  TGF157
  misc_feature (27)..(28): n is a, c, g, or t

SEQ ID NO: 111 - DNA, 25 bp, Artificial Sequence
  TGF109
cagatgggttcgggatgtactcggg

SEQ ID NO: 112 - DNA, 31 bp, Artificial Sequence
  TGF110
gaaactgcagttatagattggttgaaatccc

SEQ ID NO: 113 - DNA, 19 bp, Artificial Sequence
  MYC-ChIP-F
aagggaggcgaggatgtgt

SEQ ID NO: 114 - DNA, 19 bp, Artificial Sequence
  MYC-ChIP-F
ttcgccctggtttttccaa

SEQ ID NO: 115 - DNA, 17 bp, Artificial Sequence
  PABPC1- ChIP-F
cagcggcagtggatcga

SEQ ID NO: 116 - DNA, 24 bp, Artificial Sequence
  PABPC1- ChIP-F
caaccggaattgaaaactactcaa

SEQ ID NO: 117 - protein, 9 aa, Artificial Sequence
  PylRS partial sequence
AAANNKTTC

SEQ ID NO: 118 - protein, 9 aa, Artificial Sequence
  PylRS partial sequence
CCANNKAAA

SEQ ID NO: 119 - DNA, 39 bp, Artificial Sequence
  Py1Rs random clone
actcttagtaactatgatcgaaaaaacttcccgcagatg

SEQ ID NO: 120 - DNA, 39 bp, Artificial Sequence
  Py1Rs random clone
actcttggtaactatgctcgaaaaaacttctcgcagatg

SEQ ID NO: 121 - DNA, 39 bp, Artificial Sequence
  Py1Rs random clone
actcttaataactattggcgaaaaaacttcactcagatg

SEQ ID NO: 122 - DNA, 39 bp, Artificial Sequence
  Py1Rs random clone
actcttcagaactatagtcgaaaaaacttcatgcagatg

SEQ ID NO: 123 - DNA, 39 bp, Artificial Sequence
  Py1Rs random clone
actcttgggaactataagcgaaaaaacttcaatcagatg

SEQ ID NO: 124 - DNA, 39 bp, Artificial Sequence
  Py1Rs random clone
actcttgtgaactatgtgcgaaaaaacttcctgcagatg

SEQ ID NO: 125 - DNA, 39 bp, Artificial Sequence
  Py1Rs random clone
actcttcttaactatattcgaaaaaacttccttcagatg

SEQ ID NO: 126 - DNA, 39 bp, Artificial Sequence
  Py1Rs random clone
actcttctgaactattggcgaaaaaacttccgtcagatg

SEQ ID NO: 127 - DNA, 24 bp, Artificial Sequence
  Py1Rs random clone
actcttaggaactatgcgcgaaaa

SEQ ID NO: 128 - DNA, 39 bp, Artificial Sequence
  Py1Rs random clone
actctttataactatgctcgaaaaaacttctttcagatg

SEQ ID NO: 129 - DNA, 39 bp, Artificial Sequence
  Py1Rs random clone
actcttgggaactataagcgaaaaaacttcccgcagatg

SEQ ID NO: 130 - DNA, 39 bp, Artificial Sequence; identical to SEQ ID NO: 119
  Py1Rs random clone

SEQ ID NO: 131 - DNA, 39 bp, Artificial Sequence; identical to SEQ ID NO: 119
  Py1Rs random clone

SEQ ID NO: 132 - protein, 16 aa, Artificial Sequence
  sfGFP150 Kfu insertion
LEYNFNSHKVYITADK

SEQ ID NO: 133 - protein, 5 aa, Artificial Sequence
  V1 partial sequence
KQTAR

SEQ ID NO: 134 - protein, 10 aa, Artificial Sequence
  V1C partial sequence
KQTARKSTGG

SEQ ID NO: 135 - protein, 11 aa, Artificial Sequence
  V3C partial sequence
MKQTARKSTGG

SEQ ID NO: 136 - protein, 6 aa, Artificial Sequence
  V3 partial sequence
MKQTAR

SEQ ID NO: 137 - protein, 12 aa, Artificial Sequence
  H3 partial sequence
QTARKSTGGVSK

SEQ ID NO: 138 - protein, 5 aa, Artificial Sequence
  Scrambled H3 partial sequence
GRGTA

(19) United States
(12) Patent Application Publication
(10) Pub. No.: US 2023/0272370 A1
(43) Pub. Date: Aug. 31, 2023

(54) GENETICALLY ENCODED YEATS DOMAIN PROBE AND APPLICATIONS THEREOF

(71) Applicant: THE UNIVERSITY OF HONG KONG, Hong Kong (CN)

(72) Inventors: Xiang David LI, Hong Kong (CN); Xin LI, Hong Kong (CN); Gaofei TIAN, Hong Kong (CN)

(21) Appl. No.: 18/006,977

(22) PCT Filed: Jul. 27, 2021
(86) PCT No.: PCT/CN2021/108680

§ 371 Date: Jan. 26, 2023

Related U.S. Application Data
(60) Provisional application No. 63/057,933, filed on Jul. 29, 2020.

(51) Int. Cl. C12N 15/10; C12N 9/10; C07D 307/68; C07K 14/47; C12N 15/70; C12N 15/85

(52) U.S. Cl. CPC C12N 15/1055; C12N 9/1025; C12Y 203/02003; C07D 307/68; C07K 14/47; C12N 15/70; C12N 15/85

(57) ABSTRACT

Provided is a product, composition that contains genetically encoded YEATS domain probe. Provided is a method of making the genetically encoded YEATS domain probes. Also provided are methods of modulating YEATS domain proteins using the genetically encoded probes.

This international patent application claims the benefit of U.S. Provisional Patent Application No. 63/057,933 filed on Jul. 29, 2020, the entire content of which is incorporated by reference for all purpose.

### 1. FIELD

Provided is a product, composition that contains genetically encoded YEATS domain probe. Provided is a method of making the genetically encoded YEATS domain probes. Also provided are methods of modulating YEATS domain proteins using the genetically encoded probes.

### 2. BACKGROUND

Histone posttranslational modifications (PTMs) represent an essential mechanism for the epigenetic regulation of gene functions². Notably, histone PTM dynamics are regulated by “writer” and “eraser” proteins that catalyze the addition or removal of histone PTMs². Moreover, histone PTMs also serve as a docking site for “reader” proteins that recognize and specifically bind to them, thus signaling downstream biological events (for example, gene transcription, DNA replication and repair)³⁻⁵. Given their importance in maintaining normal cell physiology, dysregulation of these “writers”, “erasers”, or “readers” have been implicated in many human diseases, such as cancer⁶⁻⁸. Thus, it is of fundamental important to elucidate the regulatory mechanisms as well as the biological functions of histone PTMs.

The development of chemical epigenetic probes, mostly inhibitors, has provided useful tools to probe the regulatory mechanisms and biological significances of histone PTMs. For example, several histone deacetylase (HDAC) inhibitors have been used to probe the biological functions of histone lysine acetylation (Kac)⁹. Many chemical probes for Bromodomain (BrD), which recognizes Kac, have also been developed to assist the functional studies of BrDs^{10, 11}.

YEATS domain is an emerging reader module that selectively recognizes histone lysine acylation with a preference for crotonylation (Kcr) over acetylation (Kac)^{12, 13} The human genome encodes four YEATS domain containing proteins: AF9, ENL, YEATS2, and GAS41¹⁴. AF9 plays roles in embryonic development and the self-renewal of human hematopoietic stem cells^{15, 16}. YEATS domain of AF9 has been reported to mediate active transcription through ‘reading’ the histone Kac and Kcr marks, with a preference to Kcr^{12, 17, 18} ENL was reported to be essential for oncogenic gene expression in aggressive leukemia, in a YEATS domain dependent manner^{19, 20} YEATS2 was found to regulate the gene transcription in non-small cell lung cancer through the interaction between its YEATS domain and histone H3K27ac mark²¹. By reading H3K14ac and H3K27ac marks, GAS41 serves an essential role in H2A.Z deposition, which further maintains embryonic stem cell (ESC) identity and promotes cancer cell growth in non-small cell lung cancer^{22, 23}. In spite of these discoveries, a comprehensive understanding of the functional outcomes of YEATS-Kac/Kcr interactions is still lacking. Our lab has provided a series of peptide-based YEATS domain inhibitors²⁴. Based on the reported crystal structure of AF9 YEATS domain complexed with H3K9cr peptide, in which a π-π-π stacking formed by the crotonyl group and two conserved aromatic residues (Phe59 and Tyr78 in AF9) prominently contributes to the binding toward crotonylation marks¹⁷. We replaced the crotonyl group of H3K9 with an expanded n system to enhance such interaction and serve as potential YEATS domain inhibitors.

Genetic code expansion (GCE) technology, which utilizes the engineered aminoacyl-tRNA synthetase/tRNA (aaRS/tRNA) pairs to code for unnatural amino acids (uAAs), has enabled the site-specific incorporation of varying uAAs into proteins^{25, 26}. The pyrrolysyl-tRNA synthetase/pyrrolysyl-tRNA (PylRS/PyltRNA) pair, which is the most popular aaRS/tRNA pair developed in GCE, has been used for the genetic incorporation of different kinds of lysine derivatives^{27, 28}.

The genetically encoded YEATS domain probe described in the present disclosure possesses some advantages over the peptide or small molecule-based chemical probes. Such as better intracellular stability, better subcellular localization control. The present disclosure demonstrates its potential applications and advantages in the functional studies of YEATS domain proteins.

### 3. SUMMARY

In one embodiment, provided herein is a 2-furancarbonyl lysine having the formula:

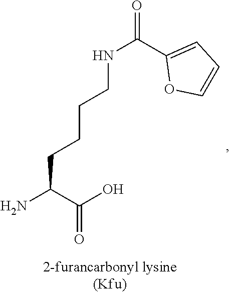 [SMILES: N[C@@H](CCCCNC(=O)c1ccco1)C(=O)O]

or salts thereof.

In one embodiment, provided herein is a method of making a polypeptide comprising a 2-furancarbonyl lysine, said method comprises translation of a RNA encoding said polypeptide, wherein said RNA comprises an amber stop codon, and wherein said translation is carried out in the presence of a tRNA charged with 2-furancarbonyl lysine and the translation terminates at the amber stop codon.

In one embodiment, the tRNA charged with 2-furancarbonyl lysine is supplied by providing a combination of tRNA capable of being charged with 2-furancarbonyl lysine, a tRNA synthetase capable of charging said tRNA with 2-furancarbonyl lysine, and in the presence of 2-furancarbonyl lysine.

In one embodiment, the tRNA synthetase capable of charging said tRNA with 2-furancarbonyl lysine comprises Methanosarcina barkeri pyrrolysyl-tRNA synthetase (MbPylRS) with three mutations relative to the wild-type sequence wherein the mutations are L274A and C313F and Y349F.

In one embodiment, the tRNA capable of being charged with 2-furancarbonyl lysine comprises Methanosarcina barkeri tRNACUA.

In one embodiment, provided herein is a polypeptide comprising: (i) a histone H3-derived decapeptide; and (ii) a partner protein that fused with the H3-derived decapeptide.

In one embodiment, the histone H3-derived decapeptide is from histone H3 residue 4-13 (K4Q5T6A7R8K9S10T11G12G13) (SEQ ID NO: 1) comprising a 2-furancarbonyl lysine at lysine 9 position.

In one embodiment, the H3-derived decapeptide is capable of binding with YEATS domains.

In one embodiment, the polypeptide comprises AF9.

In one embodiment, the partner protein comprises a superfolder GFP (sfGFP) protein.

In one embodiment, provided herein is a method of disrupting the interaction of YEATS domain of AF9 with chromatin comprises the step of expressing the polypeptide in mammalian cells.

In one embodiment, the partner protein comprises DNA binding proteins, transcription factors and dCas9 protein.

In one embodiment, the DNA binding protein comprises Lac repressor (LacR) protein.

In one embodiment, provided herein is a method of recruiting YEATS domain protein to a specific genomic locus comprising the step of expressing the polypeptide in mammalian cells comprising a genome.

In one embodiment, the YEATS domains comprise AF9.

In one embodiment, the specific genomic locus comprises Lac operator (LacO) arrays which is stably integrated into the genome of the mammalian cells.

In one embodiment, the DNA binding protein comprises GAL4 DNA binding domain (DBD).

In one embodiment, provided herein is a method of recruiting YEATS-VP64 fusion protein to the GAL4 upstream activating sequence (UAS) sequence upstream of a luciferase gene by genetically express the polypeptide in mammalian cells.

In one embodiment, the YEATS domains comprise AF9.

In one embodiment, provided herein is a vector expressing the polypeptide wherein said histone H3-derived decapeptide comprises one or more 2-furancarbonyl lysine.

In one embodiment, provided herein is a system or a cell comprising the vector.

In one embodiment, provided herein is a method of treating a disorder comprising administering the polypeptide.

In one embodiment, provided herein is a method of screening for a molecule that modulates YEATS domain proteins, said method comprises: (i) providing the vector; (ii) detecting binding between the polypeptide and a target molecule; and (iii) identifying a target molecule.

In one embodiment, the method further comprises the steps of: (i) determining whether the binding between the polypeptide and a target molecule is capable of moderating a DNA binding protein; and (ii) producing a detectable effect.

In one embodiment, the method of screening is a high throughput screening.

In one embodiment, the target molecule is further assessed as a candidate drug.

In certain embodiments, disclosed herein are proteins where 2-furancarbonyl lysine is genetically and site-specifically incorporated using an engineered PylRS/PyltRNA pair. In one embodiment, provided herein is a decapeptide (histone H3, residue 4-13, K₄Q₅T₆A₇R₈K₉S₁₀T₁₁G₁₂G₁₃) (SEQ ID NO:1) with a 2-furancarbonyl lysine at lysine 9 position exhibited a tighter binding with AF9 YEATS domain. In one embodiment, provided herein is a YEATS domain inhibitor (FIG. 1). The decapeptide, with a 2-furancarbonyl lysine site-specifically installed at lysine 9 position by PylRS/PyltRNA pair, is expressed inside the cell as a protein tag (FIG. 2). This genetically encoded protein tag, which could bind with YEATS domain, serves as a genetically encoded probe to modulate (e.g., inhibit or recruit) YEATS domain proteins when fused with different protein partners (FIG. 2).

In one embodiment, the disclosure provides a genetically encoded YEATS domain probe, that is a histone H3-derived decapeptide (H3 residue 4-13, K₄Q₅T₆A₇RK₉S₁₀T₁₁G₁₂G₁₃) (SEQ ID NO:1) bearing a 2-furancarbonyl lysine, which is co-translationally installed at K9 position by PylRS/PyltRNA pair (FIG. 2). The probe binds with YEATS domain and is used to manipulate YEATS domain proteins in cells (FIG. 2).

The use of the genetically encoded probe allows inhibition of YEATS domain-Kac/Kcr interaction. For example, when fusing the probe with superfolder green fluorescence protein (sfGFP), the fusion protein may compete with AF9 YEATS domain for chromatin Kac/Kcr mark binding, thus reduce the chromatin and genomic localization of AF9 protein (FIG. 2).

The genetically encoded YEATS domain probe can also be used to recruit YEATS domain proteins to a specific genome locus (FIG. 2). For example, when fusing the probe with LacR repressor, which binds tightly to LacO DNA sequence, the fusion protein could localize to LacO arrays that integrated into the genome and recruit AF9 protein to the same locus. Moreover, when the probe is fused with dCas9 protein, the fusion protein may target YEATS domain proteins to any positions in the genome.

Thus, in one aspect, the present disclosure provides a method of making polypeptide comprising 2-furancarbonyl lysine, wherein the method comprises the following steps:
i) introducing an amber stop codon at the desired site, or replacing a specific codon in the nucleotide sequence encoding the polypeptide;
ii) introducing the PylRS/PyltRNA expression system as described herein into a cell; and
iii) growing the cell in a medium with the 2-furancarbonyl lysine as described herein present in the medium.

A still further aspect of the present disclosure relates to the use of genetically encoded YEATS domain probe according to the disclosure in modulating (e.g., inhibiting or recruiting) YEATS domain proteins.

Provided herein is a 2-furancarbonyl lysine having the formula shown in FIG. 3a, or salts thereof.

Provided herein is a polypeptide comprising a 2-furancarbonyl lysine.

Provided herein is a method of making a polypeptide comprising a 2-furancarbonyl lysine, said method comprising translation of a RNA encoding said polypeptide, wherein said RNA comprises an amber stop codon, wherein said translation is carried out in the presence of a tRNA charged with 2-furancarbonyl lysine and the translation terminates at said amber stop codon.

In certain embodiments, the tRNA charged with 2-furancarbonyl lysine is produced by combining tRNA capable of being charged with 2-furancarbonyl lysine, a tRNA synthetase capable of charging said tRNA with 2-furancarbonyl lysine, and in the presence of 2-furancarbonyl lysine.

In certain embodiments, the tRNA synthetase capable of charging said tRNA with 2-furancarbonyl lysine comprises Methanosarcina barkeri pyrrolysyl-tRNA synthetase (MbPylRS) with three mutations relative to the wild type sequence wherein the mutations are L274A and C313F and Y349F.

In certain embodiments, the tRNA capable of being charged with 2-furancarbonyl lysine comprises Methanosarcina barkeri tRNA_{CUA}.

In certain embodiments, the polypeptide comprises: (i) a histone H3-derived decapeptide; and (ii) a partner protein that fused with the H3-derived decapeptide.

The said histone H3-derived decapeptide is from histone H3 residue 4-13 (K₄Q₅T₆A₇R₈K₉S₁₀T₁₁G₁₂G₁₃) (SEQ ID NO:1) comprising a 2-furancarbonyl lysine at lysine 9 position prepared according to the disclosed methods herein.

The said H3-derived decapeptide is capable of binding with YEATS domains.

In certain embodiments, the YEATS domains comprise AF9.

In certain embodiments, the partner protein comprises a superfolder GFP (sfGFP) protein.

Provided herein is a method of disrupting the interaction of YEATS domain of AF9 with chromatin by genetically expresses the polypeptide as disclosed herein in mammalian cells.

Provided herein is a method of controlling the subcellular localization of said polypeptide as disclosed herein by fusing a localization sequence to the C terminal of said polypeptide.

In certain embodiments, the localization sequence comprises the nuclear localization sequence (NLS) and nuclear export sequence (NES).

In certain embodiments, the partner protein comprises DNA binding proteins, such as transcription factors and dCas9 protein.

In certain embodiments, the DNA binding protein comprises a sfGFP-LacR fusion protein.

Provided herein is a method of recruiting YEATS domain protein to a specific genomic locus by genetically expressing the polypeptide as disclosed herein in mammalian cells.

In certain embodiments, the YEATS domains comprise AF9.

In certain embodiments, the specific genomic locus comprises LacO arrays sequence that integrated into the genome.

### 4. BRIEF DESCRIPTION OF THE DRAWINGS

FIG. 1 Development of peptide-based YEATS domain inhibitor by targeting π-ππ stacking in the AF9 YEATS-Kcr complex.

FIG. 2 Schematic illustrating the disclosure. The upper pane described the generation of the genetically encoded YEATS domain probe by an orthogonal PylRS/PyltRNA pair. The lower left pane described application of the genetically encoded YEATS domain probe for YEATS domain inhibition. The lower right pane described application of the genetically encoded YEATS domain probe for YEATS domain recruitment.

FIGS. 3A-3B (A) Structure of pyrrolysine and 2-furancarbonyl lysine. (B) Structure of the active site of M. mazei PylRS bound to pyrrolysine (PDB: 2Q7H). The residues shown are conserved between M. mazei PylRS and M. barkeri PylRS.

FIGS. 4A-4B PCR-based method for PylRS library construction. (A) Randomization mutations are introduced by PCR with primers carrying a degenerate codon “NNK” at mutation sites. Then, PylRS gene fragments from the PCR are joined by overlap extension PCR to facilitate the full-length PylRS gene-carrying randomization mutations. (B) Sequencing chromatogram of the PylRS gene after overlap extension PCR. The chromatogram of two representative randomized sites is shown.

FIG. 5 Alignment of the sequencing results of randomly chosen clones.

FIGS. 6A-6C Directed evolution of PylRS for Kfu. (A) Schematic diagram illustrating the double-sieve selection. (B) The conditions used in the selection. (C) Mutated residues in KfuRS.

FIGS. 7A-7B KfuRS incorporating Kfu into sfGFP150TAG protein. (A) The assessment of fluorescence for cells expressing the sfGFP150TAG gene along with KfuRS. E. coli cultures were induced in the presence of 1 mM Kcr (black) and 1 mM Kfu (gray), or in the absence of uAA (white). (B) The Coomassie Blue-stained SDS-PAGE of lysate from cultures shown in panel a. The red arrow indicates the position of the sfGFP protein. The expression of sfGFP was verified by western blotting with His tag antibody. Results are shown as mean±SD, n=3.

FIGS. 8A-8B Characterization of Kfu-containing sfGFP. (A) ESI-MS spectra of Kfu-containing sfGFP expressed and purified from E. coli. (B) The deconvolution results of panel a were calculated using UniDec. The major peak corresponded to the full-length sfGFP containing Kfu at the 150 position, while the minor peak corresponded to the first methionine removed product.

FIG. 9 Site-specific incorporation of Kfu into the sfGFP150 site was verified by the tryptic digestion of the protein followed by ESI-MS/MS analysis of the resulting peptides.

FIGS. 10A-10B Comparison of the pBK-KfuRS and pAC-sfGFP(150TAG) pair and the pEVOL-KfuRS and pBad-sfGFP(150TAG) pair. (A) Plasmid map of pBK-KfuRS, pAC-sfGFP(150TAG), pEVOL-KfuRS, and pBad-sfGFP(150TAG). (B) Comparison of the expression elements of pBK & pAC vector and pEVOL vector for KfuRS and PylT.

FIGS. 11A-11B Comparison of the Kfu incorporation efficiency of the pBK-KfuRS and pAC-sfGFP(150TAG) pair and the pEVOL-KfuRS and pBad-sfGFP(150TAG) pair. (A) Assessment of fluorescence for cells expressing the sfGFP150TAG gene from the pBK-KfuRS and the pAC-sfGFP(150TAG) pair and the pEVOL-KfuRS and the pBad-sfGFP(150TAG) pair. Cultures were expressed in the presence of 1 mM Kfu or in the absence of uAA. (B) Coomassie Blue-stained SDS-PAGE of lysate from cultures shown in panel a. Red arrows indicate the position of the sfGFP protein. Results are shown as mean±SD, n=3.

FIGS. 12A-12D Development of the genetically encoded YEATS domain inhibitor. (A) Different designs of genetically encoding the YEATS domain inhibitor. Nuclear localization sequence (NLS). “KQTARKSTGG” (SEQ ID NO:1) was from histone H3 (4-13). These constructs were all encoded by pBad vectors and used along with the pEVOL vector. (B) Assessment of fluorescence for cells expressing sfGFP150TAG, V1, or V1C in the presence of KfuRS. (C) Expression of V2C and V3C was assessed by sfGFP fluorescence. (D) Comparison of the amber suppression of sfGFP150TAG and V3 in the presence of KfuRS. Amber suppression was calculated by dividing the fluorescence from sfGFP150TAG or V3 by the wild-type sfGFP or V3C. Results are shown as mean±SD, n=3.

FIGS. 13A-13D Enhancement of V3 inhibitor expression by targeting the context effect of amber suppression. (A and B) V3 inhibitor with different codons downstream of the amber stop codon. (C) Expression of V3, V3-1, and V3-2 was assessed by sfGFP fluorescence in the presence of KfuRS. (D) Amber suppression of sfGFP150TAG, V3, V3-1, and V3-2 in the presence of KfuRS. Results are shown as mean±SD, n=3.

FIG. 14 Coomassie Blue-stained SDS-PAGE showing the purification of V3.

FIG. 15 Gel filtration chromatography presenting the purity of V3 protein.

FIGS. 16A-16B ESI-MS assessed the identity of V3 protein. (A) ESI-MS results for V3 protein expressed in the presence of 1 mM Kfu. The right panel shows the deconvolution results of the left spectrum. Peak 1 corresponds to V3 protein and peak 2 corresponds to V3 minus 2-furancarbonyl. (B) As per (A), with an additional 20 mM NAM in the expression medium.

FIGS. 17A-17D Optimization of NAM concentration. ESI-MS results for V3 expressed in the presence of 1 mM Kfu and 1 mM NAM (A), 2 mM NAM (B), 5 mM NAM (C), or 10 mM NAM (D). The right panel shows the deconvolution results of the left spectrum. Peak 1 corresponds to V3 protein and peak 2 corresponds to V3 minus 2-furancarbonyl.

FIG. 18 Site-specific incorporation of Kfu into V3 at the K9 site was verified by Lys-C digestion of the protein followed by ESI-MS/MS of the resulting peptides.

FIG. 19 V3 bind with AF9 YEATS domain. AF9 YEATS domain was incubated with recombinant V3C-sfGFP or V3-sfGFP, followed by pull-down with GFP-Trap magnetic beads. The eluates were resolved by SDS-PAGE gel, and proteins were stained by Silver staining. For the control sample, AF9 YEATS was directly incubated with GFP-Trap beads.

FIGS. 20A-20E ITC measurements for the binding affinity of V3 toward (A) AF9 YEATS domain, (C) ENL YEATS domain, (D) Gas41 YEATS domain, (E) YEATS2 YEATS domain, as well as (B) V3C toward AF9 YEATS domain. ND, not detected.

FIGS. 21A-21B Optimization of V3 expression in mammalian cells. (A) Schematics of the vectors used. PylT is the gene encoding PyltRNA, and PylT-M15 is a variant of PylT. U6 indicates the U6 promoter, CMV is the CMV promoter, MmPylRS is PylRS from Methanosarcina mazei archaea, MbPylRS is PylRS from Methanosarcina barkeri archaea, and hMbPylRS is human codon-optimized MbPylRS. All the PylRS contain the aforementioned mutations of KfuRS. (B) Assessment of fluorescence from HEK 293T cells transfected with the indicated plasmid combinations with 1:1 ratio and cultured for 24 hours with or without 1 mM Kfu. Results are shown as mean±SEM, n=3.

FIGS. 22A-22B Optimization of the plasmid ratio and plasmid amount to enhance V3 expression. (A) The total plasmid amount was fixed at 500 ng/well (24-well plate), and different ratios of c and 3 were used to transfect HEK 293T cells. The fluorescence of cell lysates was measured 24 hours after transfection. (B) The ratio of c and 3 was fixed to 1:1, and then different amounts of plasmid were used to transfect HEK 293T cells. Fluorescence was measured 24 hours after transfection. Results are shown as mean±SEM, n=3.

FIGS. 23A-23B Time course of V3 expression in mammalian cells. (A) Assessment of fluorescence from HEK 293T cells transfected with plasmids (c+3) for 24, 48, and 72 h. (B) Amber suppression efficiency at 24, 48, and 72 h. Results are shown as mean±SEM, n=3.

FIG. 24 Fluorescence imaging showing the expression of V3 in HEK 293T cells. HEK 293T cells were transfected with plasmids (c+3) and cultured with or without Kfu for 24 hours. BF (bright field), scale bar (200 μm).

FIGS. 25A-25B (A) Western blotting of HEK 293T cells expressing V3 or V3C. To achieve the equal expression level of V3C and V3, the V3C plasmid used for transfection was 30% of the amount of V3. (B) Site-specific incorporation of Kfu into V3 at the K9 site was verified by Lys-C digestion of the protein followed by ESI-MS/MS analysis of the resulting peptides.

FIG. 26 V3 expression does not affect the expression of AF9. AF9 expression in cells expressing V3 (with or without Kfu in the culture) or V3C was checked by immunoblotting with the AF9 antibody 48 hours after transfection. To obtain equal expression levels of V3C and V3, the amount of V3C plasmid used for transfection was 30% of the amount of V3.

FIG. 27 Fluorescence images of HEK 293T cells expressing V3, V3-NLS, or V3-NES. Nuclear DNA was stained with DAPI, tubulin was labeled with Alexa 594.

FIG. 28 V3 engaged with endogenous AF9. GFP-Trap pull-down was performed with HEK293T cell nuclear extracts expressing V3-sfGFP or V3C-sfGFP. The eluates were probed with AF9 and ENL antibody.

FIGS. 29A-29B Schematics of the FRAP experiment. (A) Protein is labeled by fluorescent protein (stage 1, green dots represent labeled proteins), and a small proportion of labeled proteins (proteins inside the red circle) are bleached by an intense laser (stage 2, black dots represent bleached proteins). Then, the bleached proteins will diffuse to the unbleached region and unbleached proteins will diffuse to the bleached region (stage 3), resulting in the gradual recovery of the fluorescence of the bleached region (stage 4). (B) The FRAP recovery curve is obtained by plotting the intensity of the bleached region over time. The immobile fraction, which is the protein pool that does not undergo exchange, can be obtained by subtracting the post-bleached fluorescent intensity from the pre-bleached intensity. The mobile fraction will then become the remaining fraction. The half time of equilibrium, which is the time that the fluorescence intensity takes to recover to half of the mobile fraction level, is usually indicated as t_{1/2}.

FIGS. 30A-30C V3 inhibitor binds to AF9 and reduces its chromatin localization. (A) Nuclei of HEK 293T cells transfected with plasmids encoding AF9-sfGFP (WT, V3 and V3C) or AF9(F59A)-sfGFP (F59A). For V3 and V3C, additional plasmids for V3-NLS (mCherry) or V3C-NLS (mCherry) expression were co-transfected and 2.5 μM SAHA was added during transfection. For the V3 and V3C group, only cells co-expressing sfGFP and mCherry were selected for FRAP analysis. Red circles indicate the bleached area. (B) Fluorescence recovery curve in the bleached area. Curves represent the means of each time point with at least ten cells in each group. Error bars depict the standard error of the mean. (C) Half-times of fluorescence recovery (t_{1/2}) in panel b. Bars represent the mean t_{1/2} calculated from an individual recovery curve of at least ten cells per group, and error bars depict the standard error of the mean. The p values are based on the Student's t-test. ****p<0.0001.

FIGS. 31A-31B ChIP-qPCR results indicate that V3 disrupts the genomic localization of AF9 on its target gene. (A) HEK 293T cells were transfected with plasmids expressing V3-NLS or V3C-NLS. ChIP was performed 48 h after transfection. Localization of AF9 on MYC and PABPC1 was examined by qPCR. (B) Localization of ENL on MYC was examined by ChIP-qPCR. Error bars indicate mean±SEM; n=4 for AF9 ChIP, n=2 for ENL ChIP. The p values are based on the Student's t-test. *p<0.05, NS, no significance.

FIGS. 32A-32B (A) Schematic showing the genetically encoded YEATS domain probe V3 or a control probe V3C (scramble of H3(4-13) sequence), fused with sfGFP and LacR protein. (B) schematic illustrating the recruitment of AF9-mCherry protein to LacO array by V3-sfGFP-LacR fusion protein.

FIG. 33 Immunofluorescence microscopy of U20S-LacO cells transiently transfected with pcDNA3.1-AF9-mCherry, pNEU-KfuRS and pCMV-V3-sfGFP-LacR or pCMV-V3C-sfGFP-LacR. Live cell imaging is performed 24 hours after the transfection. Arrows indicated the position of LacO arrays that were light up by GFP concentrated dots. Scale bar 10 μm.

FIG. 34A-34D Luciferase-based two hybrid assay. (A) Schematic of the Luciferase-based two hybrid assay. (B) Schematic showing V3-GAL4 and V3C-GAL4 fusion proteins. (C) Immunoblotting analysis the expression of V3-GAL4 and V3C-GAL4 in the presence or absence of 1 mM Kfu in HEK 293T cells. (D) Relative luciferase signal of V3-GAL4 or V3C-GAL4 ‘bait’ in the presence of AF9-VP64 ‘prey’ protein, and with the addition of different concentrations of iMLLT. The luminescence of all the samples was normalized to the V3C-GAL4 sample. Error bars represent SEM from three biological replicates.

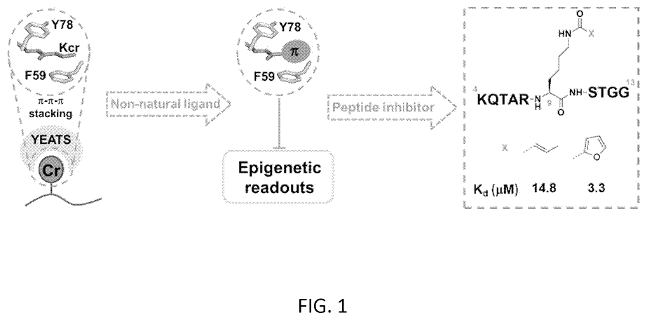

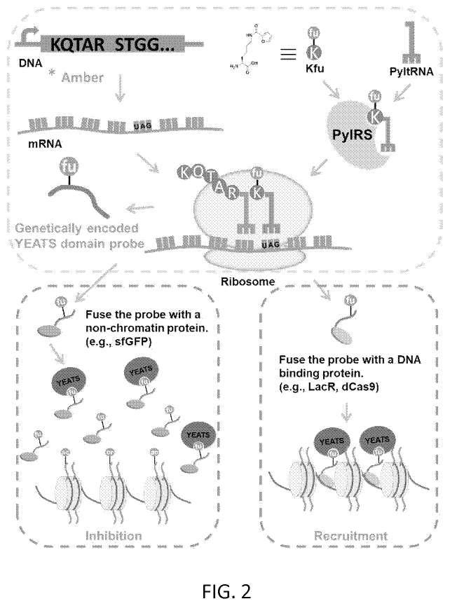

### 4.1 DEFINITION

The term “about” as used herein means within ±5% of a given value or range.

### 5. DETAILED DESCRIPTION

In one aspect, provided herein is a polypeptide that is able of regulating gene expression in mammalian cells via an epigenetic control mechanism. In one embodiment, the polypeptide is capable of providing one or more epigenetic modifications to a genome of a host cell. In one embodiment, the polypeptide is a YEATS domain probe. In one embodiment, the epigenetic modification refers to stably-heritable chromosomal alterations without alterations to the DNA sequence. Epigenetic modifications typically involve changes in the chromatin structure that can result in overexpression and/or repression of genes that control cellular processes such as differentiation, proliferation, and/or apoptosis. Such modifications can involve, e.g., DNA methylation and histone acetylation. See, e.g., Gnyszka, A., et al., Anticancer Res. 33:2989-2996 (2013). In certain embodiments, epigenetic modifications lead to treatment or prevention of a disease or disorder. In one embodiment, the disease is cancer.

In certain embodiments, the polypeptides as disclosed herein is an epigenetic modulating agent refers to an agent, e.g., a therapeutic agent, which can affect, e.g., block, reduce, reverse, or alleviate, a disease-causing epigenetic modification, thereby treating the disease, e.g., cancer.

In one aspect, provided herein is a modified amino acid residue. In one embodiment, the modified amino acid residue is a modified lysine. In one embodiment, the modified lysine is 2-furancarbonyl lysine. Also provided herein is a polypeptide that is capable of epigenetic interactions. Provided herein is a method of making the polypeptide as disclosed. The present disclosure further provides methods for modulating YEATS domain, for treating or preventing cancer in a subject, and for modulating transcription in a subject. In certain embodiments, the disclosure provides methods of recruiting YEATS domain proteins to a specific location in a genome where the polypeptide as disclosed is expressed. Also provided are methods for the cell-based evaluation and screening of YEATS domain inhibitors.

### 5.1 Polypeptides

In one aspect, provided herein is a polypeptide that is capable for epigenetic interactions. In one embodiment, the polypeptide comprises: (i) a modified histone fragment that comprises a modified amino acid residue; and (ii) a partner protein. In one embodiment, the polypeptide comprises a histone H3-derived peptide. In one embodiment, the H3-derived peptide comprises 4-8, 8-10, 10-13, 13-15, 15-20, 20-25 amino acid residues. In one embodiment, the modified amino acid residue is 2-furanocarbonyl lysine. In one embodiment, the H3-derived peptide fragment comprises amino acid residues 4-13 of the H3 protein. In one embodiment, the modified amino acid residue is at lysine 9 position of the H3 protein. In one embodiment, the polypeptide is K₄Q₅T₆A₇R₈K₉S₁₀T₁₁G₁₂G₁₃ (SEQ ID NO: 1). In one embodiment, the modified histone fragment comprising the modified amino acid residue is capable of binding to YEATS domains. In one embodiment, the polypeptide is a YEATS domain inhibitor. In one embodiment, the polypeptide is a YEATS domain probe. In one embodiment, the YEATS domain is AF9. In one embodiment, the partner protein is a non-chromatin protein. In one embodiment the partner protein is associated with a marker. In certain embodiments, the marker is DAPI, fluorescein isothiocyanate, europium (III) hydroxide, ferrocene, europium (III) chloride, beta-galactosidase, yellow fluorescent protein, fluorophore, Texas red, TRITC, firefly luciferase, fluorescent tag or a combination thereof. In one embodiment, the partner protein is a fluorescent protein. In one embodiment, the partner protein is a superfolder green fluorescent protein (“GFP”) (sfGFP), Yellow fluorescent protein (YFP), mCherry and other fluorescent proteins. In one embodiment, the partner protein is a chromatin protein. In one embodiment, the partner protein is a DNA binding protein. In one embodiment, the partner protein is a transcription factor. In one embodiment, the partner protein is dCas9. In one embodiment, the partner protein is Lac repressor protein (“LacR”). In one embodiment, the partner protein is a transcription factor. Useful transcription factors include but are not limited to Gal 4, repressor Lex A, Tet repressor proteins (TetR), heat shock factor (HSF), estrogen receptors (ERs), hypoxia inducible factor (HIF), homeobox genes, (Hox) and TATA binding proteins (GTFs).

In one embodiment, the polypeptide as disclosed herein encompasses a singular polypeptide as well as plural polypeptides, and refers to a molecule composed of monomers (amino acids) linearly linked by amide bonds (also known as peptide bonds). The term “polypeptide” refers to any chain or chains of two or more amino acids, and does not refer to a specific length of the product. Thus, in certain embodiments, the polypeptide of the present disclosure comprises peptides, dipeptides, tripeptides, oligopeptides, protein, or a chain or chains of two or more amino acids. In one embodiment, the polypeptide as disclosed herein comprises the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology, but is not necessarily translated from a designated nucleic acid sequence. In one embodiment, the polypeptide can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

In one embodiment, the polypeptide disclosed herein is an isolated polypeptide or a fragment, variant, or derivative thereof. In one embodiment, the polypeptide is a polypeptide that comprises one or more non-naturally occurring amino acid residue. In certain embodiments, no particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

In certain embodiments, other polypeptides disclosed herein are fragments, derivatives, analogs, or variants of a naturally occurring polypeptide, and any combination thereof. The terms “fragment,” “variant,” “derivative” and “analog” as disclosed herein include any polypeptides which retain at least some of the properties of the corresponding native polypeptide, for example, specifically binding to the DNA or another polypeptide. Fragments of polypeptides include, for example, proteolytic fragments, as well as deletion fragments. Variants of, e.g., a polypeptide include fragments as described above, and also polypeptides with altered amino acid sequences due to amino acid substitutions, deletions, or insertions. In certain aspects, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine or 2-furancarbonyl lysine or 5-oxazolecarbonyl lysine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the amino acid sequences of the polypeptides of the present disclosure abrogate the binding of the polypeptide containing the amino acid sequence, to which the binding molecule binds. In certain embodiments, conservative substitutions in the sequences of the polypeptides of the present disclosure do not abrogate the binding of the polypeptide containing the amino acid sequence, to which the binding molecule binds.

### 5.2 Methods of Making a Polypeptide Comprising a Modified Amino Acid Residue

In one embodiment, provided herein is an engineered aminoacyl-tRNA synthetase/tRNA (aaRS/tRNA) pairs which encode for unnatural amino acids (uAAs) and provide site-specific incorporation of varying uAAs into proteins. In one embodiment, provided herein is the pyrrolysyl-tRNA synthetase/pyrrolysyl-tRNA (PylRS/PyltRNA) pair, which is an aaRS/tRNA pair, for the genetic incorporation of lysine derivatives. In one embodiment, provided herein is a method of making a polypeptide comprising a 2-furancarbonyl lysine. In one embodiment, the polypeptide comprises a 5-oxazolecarbonyl lysine. The method comprises arranging for the translation of a RNA encoding the polypeptide wherein the RNA comprises an amber stop codon and wherein the translation is carried out in the presence of a tRNA charged with 2-furancarbonyl lysine and the translation terminates at the amber stop codon. In one embodiment, the tRNA charged with 2-furancarbonyl lysine is prepared by providing a combination of tRNA capable of being charged with 2-furancarbonyl lysine, a tRNA synthetase capable of charging the tRNA with 2-furancarbonyl lysine and in the presence of 2-furancarbonyl lysine. In one embodiment, the tRNA synthetase comprises Methanosarcina barkeri pyrrolysyl-tRNA synthetase (MbPylRS) with three mutations relative to the wild-type sequence wherein the mutations are L274A, C313F and Y349F. In one embodiment, the tRNA comprises Methanosarcina barkeri tRNA_{CUA}.

### 5.3 Nucleic Acid Molecules and Vectors

Provided herein are nucleic acid molecules and vectors that encode the polypeptides as disclosed herein.

In certain embodiments, provided herein are polynucleotides or nucleic acid molecules. In one embodiment, the nucleic acid molecules are DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse .beta.-glucuronidase.

Provided herein is a vector used as a vehicle to transfer genetic material into a cell. In one embodiment, the vector encompasses—but is not restricted to—plasmids, viruses, cosmids and artificial chromosomes. In general, engineered vectors comprise an origin of replication, a multicloning site and a selectable marker. The vector itself is generally a nucleotide sequence, commonly a DNA sequence that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences such as a promoter sequence that drives expression of the transgene. Insertion of a vector into the target cell is usually called “transformation” for bacteria, “transfection” for eukaryotic cells, although insertion of a viral vector is also called “transduction”.

In one embodiment, the polypeptide is produced by expression of a nucleic acid molecule or a vector that encodes the polypeptide. In certain embodiments, the steps involved in the production of a polypeptide described herein including, but not limited to, transcription, post-transcriptional modification, translation, post-translational modification, and secretion.

In one embodiment, the nucleic acid molecule or vector comprises control sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A nucleic acid molecule comprises nucleic acid sequences that are operably linked when it is placed into a functional relationship with one another. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Provided herein are host cells into which a nucleic acid encoding the polypeptide described herein is introduced by way of transformation, transfection and the like. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. In certain embodiments, the host cell includes any individual cell or cell culture that can be or has been recipients for vectors or the incorporation of exogenous nucleic acid molecules, polynucleotides, vectors and/or proteins. The cells may be prokaryotic or eukaryotic, and include but are not limited to bacteria, yeast cells, animal cells, and mammalian cells, e.g., murine, rat or human. Suitable eukaryotic host cells include yeasts, fungi, insect cells and mammalian cells.

### 5.4 Therapeutic Agent

The disclosed polypeptide, variant or derivative thereof can be useful as an epigenetic modulating agent. Administration of the epigenetic modulating agent can be useful for the treatment of various malignant and non-malignant tumors. By “anti-tumor activity” is intended a reduction in the rate of malignant cell proliferation or accumulation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. “Anti-tumor activity” can also comprise promotion of immune infiltration into the tumor, a shift toward functional, tumor-specific, IFNγ-secreting CD8⁺ cytotoxic T cells, an increase in the ratio of T effector cells to T regulatory cells, increased T-cell activity, infiltration and activation of antigen presenting cells that cross-present tumor antigens and locally activate tumor-specific T cells, reduced tumor-associated angiogenesis, inhibition of tumor progression, and enhanced survival. For example, therapy with an epigenetic modulating agent can elicit a physiological response, for example, inhibition, delay or reduction of tumor or malignant cell growth and metastases, which is beneficial with respect to treatment of cancer.

In certain aspects, combination therapy with an epigenetic modulating agent can be used as a medicament, in particular for use in the treatment or prophylaxis of cancer or for use in a precancerous condition or lesion. In certain aspects, combination therapy with an epigenetic modulating agent can be used for the treatment of cancer.

In accordance with the treatment methods provided herein, administration of an epigenetic modulating agent can be used to promote a positive therapeutic response in the subject with cancer or predisposed to contract cancer. A “positive therapeutic response” with respect to cancer is intended to include an improvement in the disease in association with the “anti-tumor” activity is intended a reduction in the rate of malignant cell proliferation or accumulation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such positive therapeutic responses are not limited to the route of administration. The methods provided herein can be drawn to inhibiting, delaying, or reducing tumor growth, malignant cell growth, and metastases in a subject with cancer. Thus, as a non-limiting example, an improvement in the disease can be characterized as a reduction in tumor growth or absence of tumors. As described elsewhere herein, the therapeutic response achieved upon administration of the provided combination therapy can be greater than the corresponding therapeutic response achieved upon administering an epigenetic modulating agent. In certain aspects the therapeutic response achieved is greater than the additive response expected upon administration of the two agents. In other words, the therapeutic response achieved is synergistic. In certain aspects, the synergistic response can result in a more effective treatment, a faster treatment, or can allow treatment with reduced dosages of the agents in the combination therapy.

### 5.5 Pharmaceutical Composition

In one embodiment, the composition comprises a polypeptide as disclosed herein. In one embodiment, the composition comprises a nucleic acid or a vector that expresses the polypeptide as disclosed herein. In one embodiment, provided herein is a composition disclosed herein for administration to a patient, preferably a human patient. In one embodiment, the composition comprises a YEATS domain targeting polypeptide. In one embodiment, the composition is a pharmaceutical composition comprising suitable formulations of carriers, stabilizers and/or excipients. In one embodiment, the pharmaceutical composition comprises a composition for parenteral, transdermal, intraluminal, intraarterial, intrathecal and/or intranasal administration or by direct injection into tissue. In one embodiment, the composition is administered to a patient via infusion or injection. Administration of the suitable compositions may be effected by different ways, e.g., by intravenous, intraperitoneal, subcutaneous, intramuscular, topical or intradermal administration. In particular, the present disclosure provides for an uninterrupted administration of the suitable composition. As a non-limiting example, uninterrupted, i.e. continuous administration may be realized by a small pump system worn by the patient for metering the influx of therapeutic agent into the body of the patient.

The continuous administration may be transdermal by way of a patch worn on the skin and replaced at intervals. One of skill in the art is aware of patch systems for drug delivery suitable for this purpose. It is of note that transdermal administration is especially amenable to uninterrupted administration, as exchange of a first exhausted patch can advantageously be accomplished simultaneously with the placement of a new, second patch, for example on the surface of the skin immediately adjacent to the first exhausted patch and immediately prior to removal of the first exhausted patch.

The compositions may further comprise a pharmaceutically acceptable carrier. Examples of suitable pharmaceutical carriers are well known in the art and include solutions, e.g. phosphate buffered saline solutions, water, emulsions, such as oil/water emulsions, various types of wetting agents, sterile solutions, liposomes, etc. Compositions comprising such carriers can be formulated by well-known conventional methods. Formulations can comprise carbohydrates, buffer solutions, amino acids and/or surfactants. Carbohydrates may be non-reducing sugars, preferably trehalose, sucrose, octasulfate, sorbitol or xylitol. In general, as used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counter-ions, such as sodium, polyhydric sugar alcohols; amino acids, such as alanine, glycine, asparagine, 2-phenylalanine, and threonine; sugars or sugar alcohols, such as trehalose, sucrose, octasulfate, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; sulfur containing reducing agents, such as glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Such formulations may be used for continuous administrations which may be intravenuous or subcutaneous with and/or without pump systems. Amino acids may be charged amino acids, preferably lysine, lysine acetate, arginine, glutamate and/or histidine. Surfactants may be detergents, preferably with a molecular weight of >1.2 KD and/or a polyether, in one embodiment, with a molecular weight of >3 KD. Non-limiting examples for detergents are Tween 20, Tween 40, Tween 60, Tween 80 or Tween 85. Non-limiting examples for polyethers are PEG 3000, PEG 3350, PEG 4000 or PEG 5000. Buffer systems used in the present disclosure can have a preferred pH of 5-9 and may comprise citrate, succinate, phosphate, histidine and acetate.

In one embodiment, the compositions of the present disclosure can be administered to the subject at a suitable dose which can be determined e.g. by dose escalating studies by administration of increasing doses of the polypeptide described herein exhibiting cross-species specificity described herein to non-chimpanzee primates, for instance macaques. As set forth above, the composition described herein can be advantageously used in identical form in preclinical testing in non-chimpanzee primates and as drug in humans. The composition can also be administered in combination with additional other proteinaceous and non-proteinaceous drugs. These drugs may be administered simultaneously with the composition described herein as defined herein or separately before or after administration in timely defined intervals and doses. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like. In addition, the composition of the present invention might comprise proteinaceous carriers, like, e.g., serum albumin or immunoglobulin, preferably of human origin. In certain embodiments, the composition comprises, in addition to the polypeptide described herein defined herein, further biologically active agents, depending on the intended use of the composition. Such agents might be drugs acting on the gastro-intestinal system, drugs acting as cytostatica, drugs preventing hyperuricemia, drugs inhibiting immunoreactions (e.g. corticosteroids), drugs modulating the inflammatory response, drugs acting on the circulatory system and/or agents such as cytokines known in the art. In one embodiment, the composition comprises the polypeptide as disclosed herein in a formulations is applied in an additional co-therapy, i.e., in combination with another medicament. In one embodiment, the medicament is an anti-cancer drug.

### 5.6 Cancer Therapy

In one aspect, provided herein is cancer therapy. Most if not all cancers undergo epigenetic changes, including significantly the down-regulation and silencing of tumor suppressor genes and the up-regulation of oncogenes. Reactivation of tumor suppressor genes can ameliorate cancer phenotype as can down-regulation of oncogenes. Hence, a method of controlling gene expression and cell fate decisions in vivo is a very promising avenue to cancer therapy. In certain aspects, the subject's cancer is a solid tumor or metastasis thereof. The solid tumor can be, e.g., a sarcoma, a carcinoma, a melanoma, any metastases thereof, or any combination thereof. Examples of solid tumors that can be treated according to the method provided herein include, without limitation, squamous cell carcinoma, adenocarcinoma, basal cell carcinoma, renal cell carcinoma, ductal carcinoma of the breast, soft tissue sarcoma, osteosarcoma, melanoma, small-cell lung cancer, non-small cell lung cancer, adenocarcinoma of the lung, cancer of the peritoneum, hepatocellular carcinoma, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, any metastases thereof, or any combination thereof.

In certain aspects, the cancer is a hematologic malignancy or metastasis thereof. Examples of hematologic malignancies that can be treated according to the method provided herein include, without limitation, leukemia, lymphoma, myeloma, acute myeloid leukemia, chronic myeloid leukemia, acute lymphocytic leukemia, chronic lymphocytic leukemia, hairy cell leukemia, Hodgkin lymphoma, non-Hodgkin lymphoma, multiple myeloma, any metastases thereof, or any combination thereof.

In certain aspects, the cancer treatment method provided herein can further include administration of an additional cancer therapy. The additional cancer therapy can take place simultaneously with the administration of combination therapy provided herein, before the combination therapy provided herein, or after the combination therapy provided herein. In certain aspects the additional therapy can include, without limitation, surgery, chemotherapy, radiation therapy, administration of a cancer vaccine, administration of an immunostimulatory agent, adoptive T cell or antibody therapy, administration of an immune checkpoint blockade inhibitor, administration of a regulatory T cell (Treg) modulator, or a combination of such therapies.

### 5.7 Methods of Treatment

In one aspect, the present disclosure teaches a method of treatment for a disorder or disease. In certain embodiments, the method comprises administration of a therapeutically effective amount of the disclosed polypeptide, nucleic acid molecule or a vector to a host cell or a subject. In one embodiment, the treatment refers to therapeutic measures that cure, slow down, lessen symptoms of, and/or halt or slow the progression of an existing diagnosed pathologic condition or disorder. In one embodiment, the method is a prevention of a disorder or disease refers to prophylactic or preventative measures that prevent the development of an undiagnosed targeted pathologic condition or disorder. In one embodiment, the treatment or prevention is for a subject that is in need which can include those already with the disorder; those prone to have the disorder; and those in whom the disorder is to be prevented.

The method comprises of administering a therapeutically effective amount of a drug effective to treat a disease or disorder in a subject or mammal. In the case of cancer, the therapeutically effective amount of the drug can reduce the number of cancer cells; retard or stop cancer cell division, reduce or retard an increase in tumor size; inhibit, e.g., suppress, retard, prevent, stop, delay, or reverse cancer cell infiltration into peripheral organs including, for example, the spread of cancer into soft tissue and bone; inhibit, e.g., suppress, retard, prevent, shrink, stop, delay, or reverse tumor metastasis; inhibit, e.g., suppress, retard, prevent, stop, delay, or reverse tumor growth; relieve to some extent one or more of the symptoms associated with the cancer, reduce morbidity and mortality; improve quality of life; or a combination of such effects. To the extent the drug prevents growth and/or kills existing cancer cells, it can be referred to as cytostatic and/or cytotoxic.

In one embodiment, the subject for the treatment or prevention is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears etc.

### 5.8 Methods for Disrupting Interaction of Yeats Domain

In one aspect, provided herein is a method to regulate gene expression in mammalian cells via an epigenetic control mechanism. In one embodiment, the disclosure provides a method to disrupt the interaction of YEATS domain. In one embodiment, the method comprises expressing a vector. In one embodiment, the vector comprises a histone H3 fragment, sfGFP protein, His tag and an amber stop codon. In one embodiment, the amber stop codon is located at the histone H3 K9 position. In one embodiment, the codon following lysine 9 TCC is replaced with AGT. In one embodiment, the vector is V3-2. In one embodiment, the method comprises expressing a separate vector comprises a PylRS/PyltRNA pair. In one embodiment, the vector comprises at least one PylRS/PyltRNA pair and V3-2. In one embodiment, the vector comprises a human codon-optimized hMbPylRS gene under the control of the CMV promoter and PylT-M15 gene. In one embodiment, the PylT-M15 gene contains A10G, U14G, U16G, U20C, U25C and A52C under the control of U6 promoter. In one embodiment, the vector is construct c. In one embodiment, the vector is construct 3. In one embodiment, the method comprises expressing construct c and construct 3 in the ratio of 2:3. In one embodiment, the method comprises expressing a polypeptide as described in the disclosure. In one embodiment, the polypeptide is V3. In one embodiment, the polypeptide comprising a histone fragment that is modified with a non-natural amino acid residue and a partner protein. In one embodiment, the partner protein is a non-chromatin protein. In one embodiment, the partner protein is a marker. In one embodiment, the marker is identified by fluorescence. In one embodiment, the partner protein is GFP. In one embodiment, the partner protein comprises 5-10, 10-20, 20-30 amino acid residues. In one embodiment, the histone fragment is a H3-derived decapeptide comprising a 2-furancarbonyl lysine. In one embodiment, the polypeptide is a histone fragment analogue. In one embodiment, the histone H3-derived decapeptide is from histone H3 residue 4-13 comprising a 2-furancarbonyl lysine at lysine 9 position. In one embodiment, the H3-derived decapeptide is capable of binding to YEATS domains. In one embodiment, the YEATS domain comprises AF9. In one embodiment, the partner protein comprises a superfolder GFP (sfGFP) protein.

### 5.9 Methods for Recruiting Yeats Domain Protein to a Specific Genomic Locus

In one embodiment, the disclosure provides a method of recruiting YEATS domain protein to a specific genomic locus. In one embodiment, the method comprises expressing a vector. In one embodiment, the vector comprises a YEATS domain probe, such as a histone H3 fragment, and a lacO-lacR tethering system. In one embodiment, the vector comprises a YEATS domain probe, a sfGFP protein and a lacR repressor. In one embodiment, the vector comprises a V3-sfGFP-lacR and AF9-mCherry. In one embodiment, the V3-sfGFP-lacR and AF9-mCherry are on separate vectors. The method comprises expressing a polypeptide comprising a histone fragment that is modified with a non-natural amino acid residue and a partner protein that is a chromatin protein. In one embodiment, the partner protein is a nucleic acid binding protein. In one embodiment, the partner protein is a DNA binding protein. In one embodiment, the partner protein is a transcription factor. In one embodiment, the partner protein is GFP and LacR. In one embodiment, the partner protein is dCas9 or LacR protein. In one embodiment, the partner protein is GAL4 DNA binding domain (DBD). In one embodiment, the YEATS domain protein is AF9. In one embodiment, the histone fragment is a H3-derived decapeptide comprising a 2-furancarbonyl lysine. In one embodiment, the method comprises recruiting YEATS-VP64 fusion protein to the GAL4 upstream activating sequence (UAS) upstream of a luciferase gene. In one embodiment, the polypeptide is expressed in mammalian cells.

### 5.10 Methods of Screening for Binding Molecules

In one aspect, provided herein is a method of screening molecules for binding to the polypeptides as disclosed. The method can be performed via automated high-throughput screening procedures. The disclosure provides methods for identifying complex interacting molecules via detection of a positive binding interaction between the disclosed polypeptide and a target molecule. Further screening steps may be used to determine whether the identified positive binding interaction is of pharmacological importance—i.e. whether the target molecule is capable of moderating YEATS domain and/or YEATS domain complex biological activity or function. If a molecule with a positive moderating effect is identified, the molecule is classified as a ‘hit’ and can then be assessed as a potential candidate drug. Additional factors may be taken into consideration at this time or before, such as the absorption, distribution, metabolism and excretion, bio-availability and toxicity profiles of the molecule, for example. If the potential drug molecule satisfies the pharmacological requirements it is deemed to be pharmaceutically compatible. Suitable compositions can be formulated for testing the activity in-vitro and in-vivo in accordance with standard procedures known in the art. In one embodiment, provided is a method of screening for a molecule that modulates YEATS domain proteins, said method comprises: (i) providing a vector as disclosed herein; (ii) detecting binding between the polypeptide and a target molecule; and (iii) identifying a target molecule. In one embodiment, the method further comprises the steps of: (i) determining whether the binding between the polypeptide and a target molecule is capable of moderating a DNA binding protein; and (ii) producing a detectable effect.

### 6. EXAMPLES

### 6.1 Genetically Incorporate Kfu into Protein Via PylRS/PyltRWA Pair

Amber codon suppression via the PylRS/PyltRNA pair enables site-specifically incorporated different unnatural amino acids (uAAs) into proteins in both E. coli and mammalian cells^{25, 27, 29}. The key to genetically inserting a uAA into protein involves finding PylRS mutants catalyzing the conjunction of amber-decoding PyltRNA with that uAA and directing its incorporation at an amber stop codon. PylRS is a highly promiscuous enzyme whose specificity could be manipulated by directed evolution to coordinate structurally divergent uAAs.

### 6.1.1 Construction of MbPylRS Library

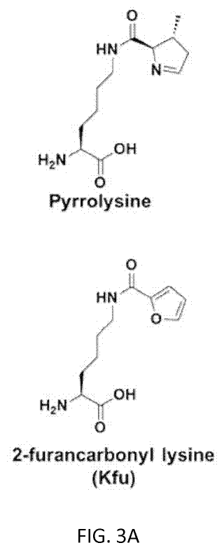

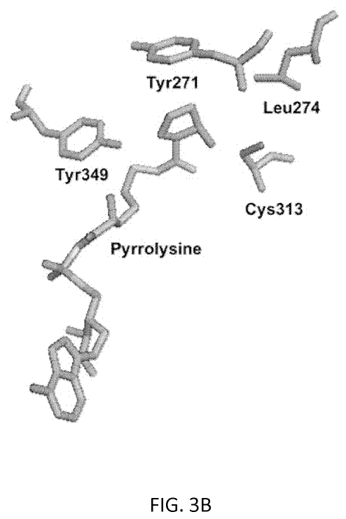

To screen for PylRS mutants that are specific to Kfu, I generated a library of M. barkeri PylRS (MbPylRS) mutants in which three residues surrounding the pyrroline ring of pyrrolysine (Tyr271, Leu274, and Cys313) were randomized. Furthermore, Tyr349 residue was fixed to Phe, which was reported to enhance the aminoacylation activity of MbPylRS. These residues were chosen based on the crystal structure of PylRS bound to adenylated pyrrolysine (FIG. 3b).

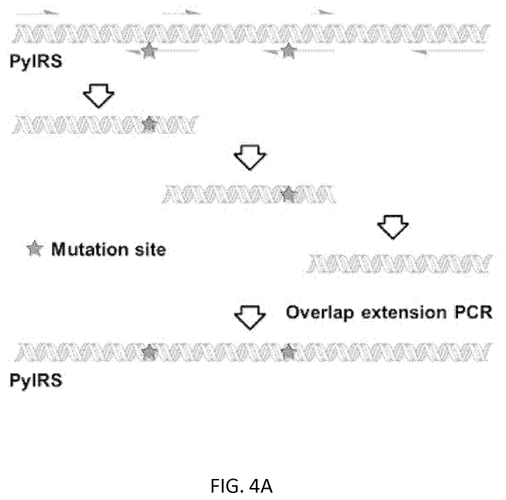

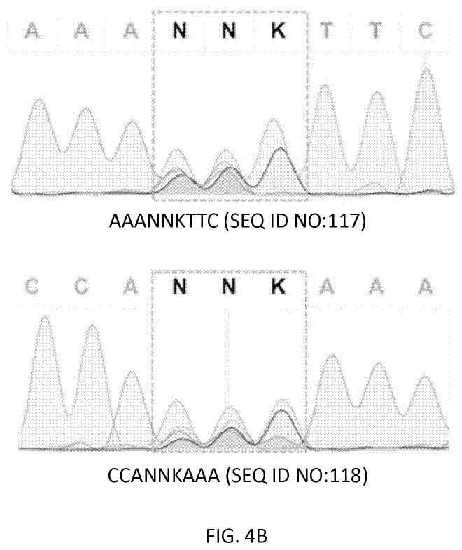

An overlapping PCR-based method was used to introduce the randomization mutation to these residues³⁰. As shown in FIG. 4a, the codon of randomization sites was replaced by the degenerate codon “NNK” (N represents A, T, C, or G, and K represents G or T), which can code for all 20 genetically encoded amino acids. Three pairs of primers carrying the degenerate codon “NNK” at mutation sites were used for PCR, and the full-length MbPylRS gene fragment was obtained by overlapping individual DNA fragments from the PCR. The quality of the full-length MbPylRS gene fragment was then examined by Sanger sequencing (FIG. 4b).

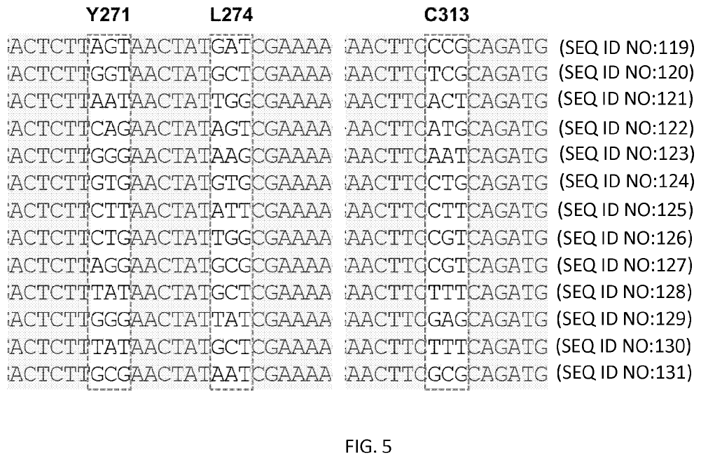

As expected, multiple peaks were observed at the randomization sites from the sequencing chromatogram. Then, the MbPylRS gene fragment was double digested and ligated into the pBK vector and used to transform DH10B competent cells. Transformants from the transformation were combined and amplified. Then, plasmids encoding the MbPylRS library were extracted from E. coli cells. The quality of the library was verified by sequencing randomly chosen clones (FIG. 5), and sequencing results showed no bias in the randomized residues. The library contains approximately 3.6×10⁵ individual transformants, which is 11-fold the size of the theoretical library (˜3.3×10⁴ PylRS mutants), thus ensuring full library coverage.

### 6.1.2 Directed Evolution of MbPylRS for Kfu

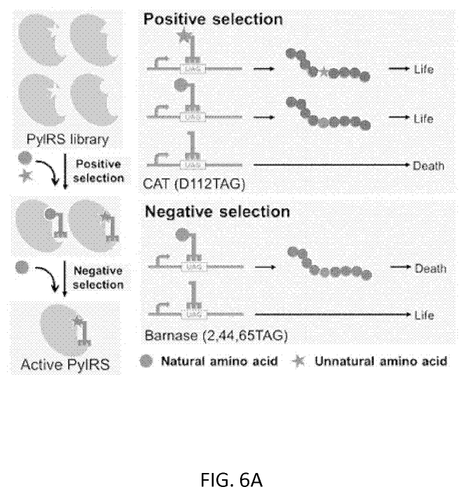

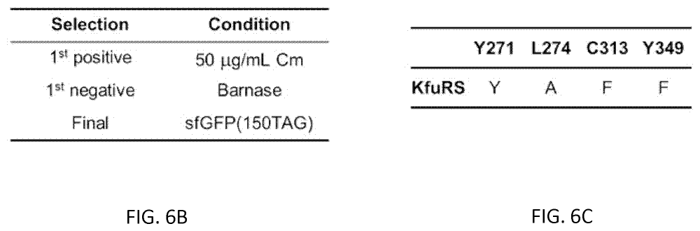

To evolve the MbPylRS mutants toward Kfu from the library, we adopted the well-developed double-sieve selection method³¹ (FIG. 6a), in which positive selection is based on the chloramphenicol acetyltransferase (CAT) antibiotic gene. An amber stop codon is inserted at a permissive aspartic acid 112 (D112TAG) position. The MbPylRS library was first transformed into E. coli cells bearing the positive selection plasmid. E. coli cells containing MbPylRS mutants that failed to incorporate a Kfu or natural amino acid at the CAT (D112TAG) position will generate a truncated inactive CAT and die during positive selection. Cells with MbPylRS mutants that can acylate the PyltRNA with either Kfu or an endogenous amino acid will survive during positive selection.

Plasmids encoding active MbPylRS mutants passed the positive selection and were then transformed into E. coli cells bearing the negative selection plasmid. Selections were then performed in the absence of Kfu. Negative selection is based on the presence of a toxic barnase gene, which encodes a nuclease that kills cells. Three amber stop codons are introduced at the glutamine 2 (Q₂TAG), aspartic acid 44 (D44TAG), and glycine 65 (G65TAG) positions. Then, E. coli cells containing MbPylRS mutants that utilized natural amino acids and read through the amber stop codons will generate the full-length toxic barnase protein and die. This double-sieve selection leads to the isolation of the MbPylRS mutant that can specifically incorporate Kfu in response to the amber stop codon.

Typically, it takes three to five rounds of positive and negative selection to gradually enrich the MbPylRS mutants that specifically utilize the unnatural amino acid from the library. One round of positive selection followed by one round of negative selection and employed a non-lethal GFP-based method to evaluate the efficiency of MbPylRS mutants that resulted from the selection³² (FIG. 6b).

In the GFP-based selection, MbPylRS mutants were allowed to suppress an amber stop codon at the sfGFP asparagine 150 (sfGFP150TAG) position in the presence of Kfu. E. coli colonies showing high sfGFP signal brightness under 365 nm UV irradiation were selected and sent for sequencing. The sequences of MbPylRS mutants from 61 colonies all converged to a unique clone, KfuRS (Tyr271, Leu274Ala, Cys313Phe, Tyr349Phe) (FIG. 6c). Interestingly, the same MbPylRS mutant has been reported to incorporate epsilon-N-crotonyllysine (Kcr) into proteins³³. For convenience, KfuRS will be used from this point forward to denote the MbPylRS mutant (Tyr271, Leu274Ala, Cys313Phe, Tyr349Phe).

### 6.1.3 Characterization of KfuRS

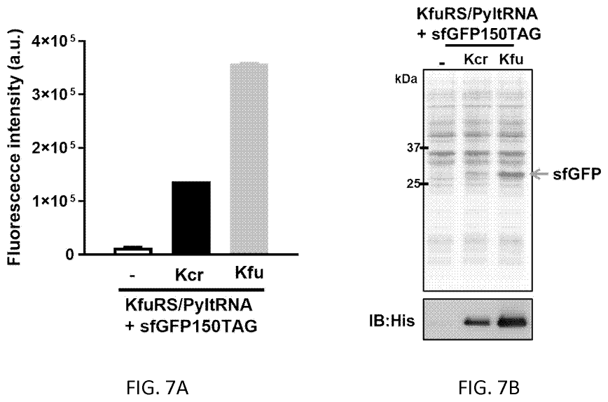

To assess the specificity and fidelity of the KfuRS/PyltRNA pair, E. coli cells containing the KfuRS/PyltRNA pair and sfGFP150TAG reporter were cultured with or without Kfu, and the sfGFP fluorescence of the culture was measured using a plate reader. As shown in FIG. 7a, sfGFP fluorescence can only be detected in the presence of Kfu, but not in the absence of Kfu, thus demonstrating the specificity of KfuRS. Results from fluorescence measurement were also verified by the Coomassie Blue-stained SDS-PAGE of E. coli cell lysates and western blotting of the lysate with His tag antibody (FIG. 7b).

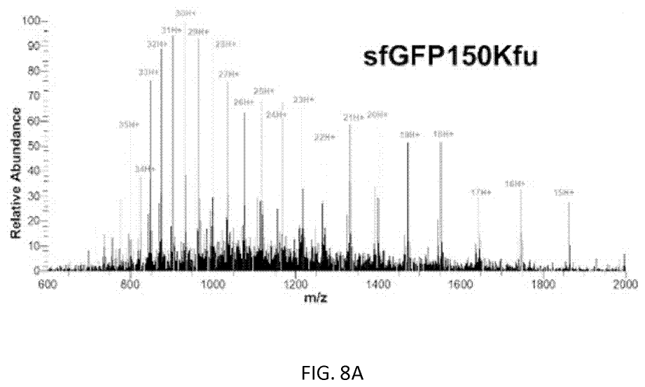

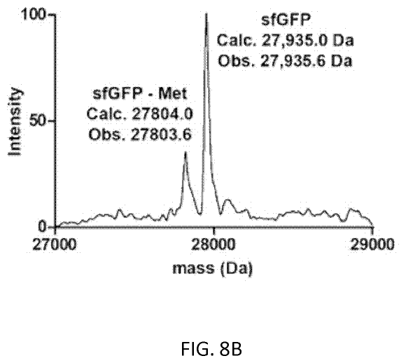

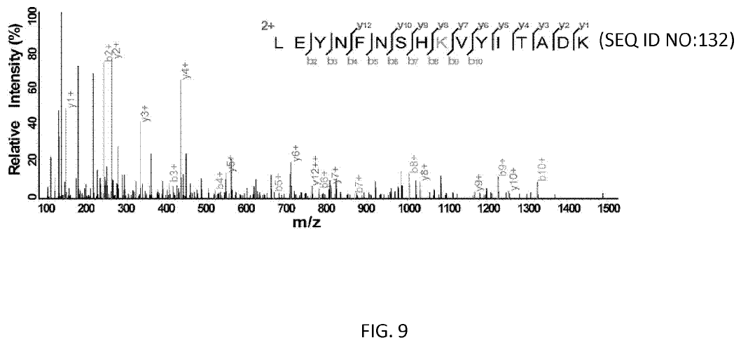

The sfGFP150TAG protein is expressed in the presence of the KfuRS/PyltRNA pair and 1 mM Kfu and then purified the protein using a Ni-NTA agarose column. Electrospray ionization (ESI)-MS mass spectra of the purified sfGFP protein demonstrate the incorporation of a single Kfu (FIGS. 8a and b). MS/MS analysis of a tryptic peptide from the sfGFP protein demonstrates the site-specific incorporation of Kfu at the sfGFP150 position (FIG. 9).

Since the KfuRS mutant was also reported to incorporate Kcr into protein, I compared the incorporation efficiency of KfuRS toward Kfu and Kcr. The results showed that although the KfuRS mutant was first reported for Kcr incorporation, it was 2.5-fold more active toward Kfu (FIG. 7a). It is also necessary to mention that poly-specificity is a common feature of PylRS. Different PylRS mutants may incorporate the same unnatural amino acid, and a single PylRS mutant may act on diverse unnatural amino acids^{34, 35}. Both Kfu and Kcr are from exogenous sources and do not naturally exist inside the cell; therefore, the poly-specificity of KfuRS will not cause heterogenous expression of protein in practice.

### 6.1.4 Optimization of KfuRS/PyltRNA Pair Amber Suppression in E. coli

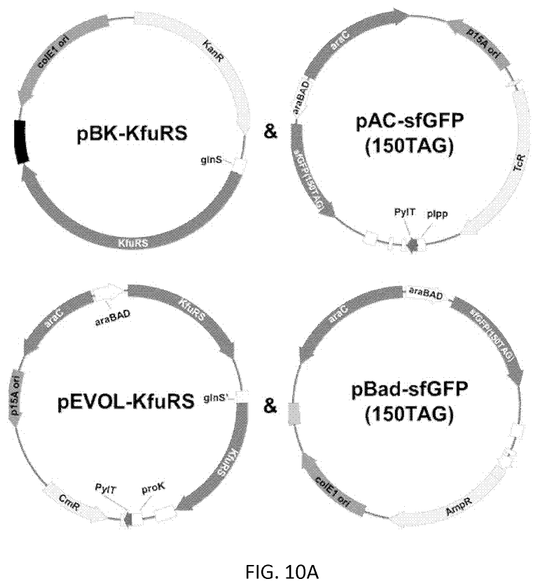

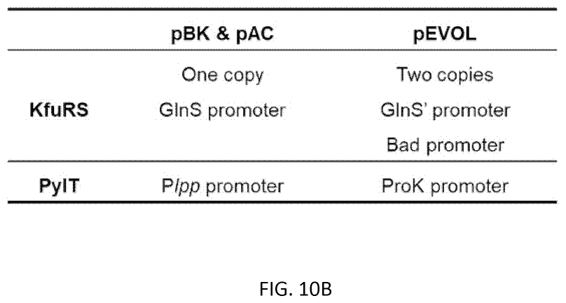

In the aforementioned experiments, the PylRS gene was encoded by a pBK-PylRS vector. The expression of the PylRS gene was under the control of the constitutive glnS promotor and terminator. Moreover, PyltRNA was under the control of the plpp promoter and rrnC terminator and encoded by the positive selection plasmid (pRep-PylT), negative selection plasmid (pAC-Bar), and GFP selection plasmid (pAC-sfGFP150TAG) (FIG. 10a). However, the amber suppression efficiency of these constructs is far from optimal. Thus, it is necessary to optimize the expression of the PylRS/PyltRNA pair to maximize the expression of the target proteins, especially for PylRS mutants with low efficiency.

To enhance the performance of Methanocaldococcus jannaschii aminoacyl tRNA synthetase (aaRS)/suppressor tRNA pairs in E. coli, in one embodiment, provided herein is a vector encodes two copies of the M. jannaschii aaRS gene: one under the control of a constitutive modified glnS′ promoter and another under the control of an inducible araBAD (arabinose induce) promoter. The expression of M. jannaschii suppressor tRNA is driven by a stronger proK promoter and terminator. These improvements increase the expression of both aaRS and suppressor tRNA, thus afford much higher amber suppression efficiency and protein yields. Moreover, unlike the pBK and pAC vector that encode the aaRS and suppressor tRNA separately, pEVOL vector encodes them on a single plasmid, enabling it's convenient application couple with the commonly used vectors for protein expression (e.g., pBad vector). A comparison between the pBK and pEVOL vectors is presented in FIG. 10b. I reasoned that these improvements should also apply to KfuRS/PyltRNA pair.

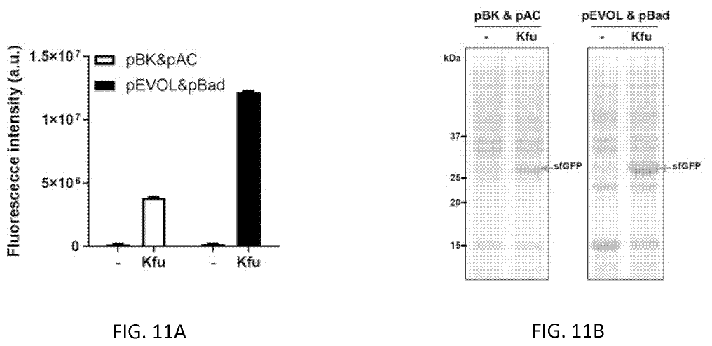

The PyltRNA and KfuRS gene is sequentially cloned into the pEVOL vector and compared the efficiency of pBK with the pEVOL vector. E. coli cells co-transformed pBK-KfuRS and pAC-sfGFP150TAG or pEVOL-KfuRS, and pBad-sfGFP150TAG were cultured. Thereafter, protein expression was induced by arabinose. sfGFP fluorescence from the culture was measured using a plate reader. The results indicate that the pEVOL-KfuRS and pBad-sfGFP150TAG pair results in a three-fold enhancement of sfGFP protein expression (FIG. 11a). The expression of sfGFP from these two pairs was also assessed by the Coomassie Blue-stained SDS-PAGE of E. coli cell lysates (FIG. 11b). Therefore, the pEVOL-KfuRS vector was used for Kfu incorporation in E. coli in subsequent studies.

### 6.2 Apply the Genetically Encoded YEATS Domain Probe for YEATS Domain Inhibition

### 6.2.1 Development of the Genetically Encoded YEATS Domain Inhibitor

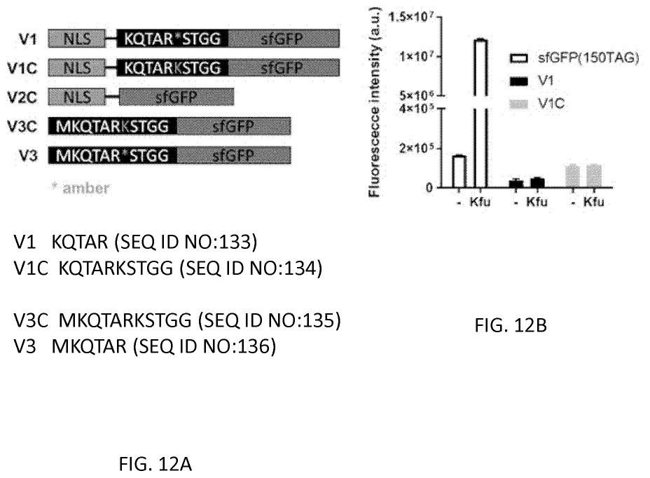

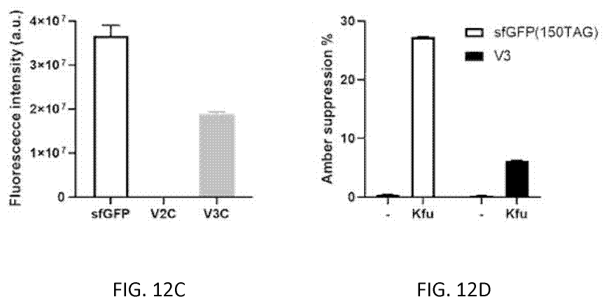

With the highly efficient KfuRS in hand, I next sought to design the genetically encoded YEATS domain inhibitor and test Kfu incorporation into it. The initial design of the genetically encoded YEATS domain inhibitor is presented in FIG. 12a (V1). A nuclear localization sequence (NLS, KRPAATKKAGQAKKKKL (SEQ ID NO:2)) from the nucleoplasmin protein was linked to the amino-terminal (N-terminal) of histone H3 (4-13) by a flexible “GGGG” (SEQ ID NO:3) linker. Then, sfGFP protein was directly fused to the carboxyl-terminal of histone H3 (4-13) and a 6×His tag was also fused to the carboxyl-terminal (C-terminal) of sfGFP to facilitate protein purification. Since all the YEATS domain-containing proteins localize in the nucleus, the N-terminal NLS will make the genetically encoded inhibitors concentrated in the cell nucleus, which enhances the inhibitory effect of the genetically encoded YEATS domain inhibitor when expressed in mammalian cells. Moreover, the fusion of sfGFP to the C-terminal of histone H3 (4-13) peptide increased the size of the inhibitor to a reasonable size for expression inside the cell and also facilitated the detection of the inhibitor. In the V1 inhibitor, the codon of H3 K9 was replaced by an amber stop codon TAG to facilitate the site-specific incorporation of Kfu. A control inhibitor with a normal codon of lysine at the H3 K9 position (VIC) was also constructed (FIG. 12a).

Then, I tested the expression of the inhibitor V1 and control inhibitor VIC in E. coli. Since the C-terminal sfGFP could only be expressed when the amber stop codon was suppressed, sfGFP fluorescence was used as an indicator for the successful expression of the inhibitor. However, no expression of V1 inhibitor in the presence of KfuRS/PyltRNA pair and Kfu was observed. Moreover, VIC also failed to express (FIG. 12b). Given that the sfGFP protein alone had a very robust expression, I reasoned that the N-terminal NLS or H3 (4-13) sequence might cause the failure of V1 and V1C expression.

In order to resolve this problem, another two versions of control inhibitors were constructed (FIG. 12a). V2C was constructed by fusing NLS to the N-terminal of sfGFP, while V3C was constructed by fusing H3 (4-13) to the N-terminal of sfGFP. These two control inhibitors were then expressed in E. coli. The results indicate that V3C has robust expression, but V2C fails to express (FIG. 12c). Failure in V2C expression indicated that the N-terminal NLS sequence might be unfavorable for translation initiation in E. coli. Since the NLS sequence only functions in mammalian cells and is dispensable when expressed in E. coli, I removed the NLS sequence and “GGGG” linker from the V1 inhibitor to generate the V3 inhibitor.

The expression of V3 was tested in the presence of the KfuRS/PyltRNA pair and Kfu. The results indicate that the V3 inhibitor could only express in the presence of Kfu (FIG. 12d). I also compared the amber suppression efficiency of V3 and sfGFP150TAG. The amber suppression efficiency of V3 was 6%, which was much lower than that of sfGFP150TAG (27%) (FIG. 12d).

### 6.2.2 Enhance the Expression of V3 Inhibitor by Targeting the Context Effect of Amber Suppression

Based on previous results, I observed that the amber suppression of V3 was much lower than that of sfGFP150TAG (6% vs. 27%, respectively). This difference in amber suppression efficiency is attributed to the context effects of amber suppression. Context effects were first observed and defined when studying how neighboring codons influence natural stop codon suppressors in E. coli⁴¹⁻⁴³. Several systematic studies on the context effect of amber suppression revealed that the identity of nucleotides downstream of the amber stop codon had the most striking influence on amber suppression efficiency. This influence follows the order A>G>C>U, with nucleotide A downstream of the amber stop codon providing the highest level of amber suppression, while nucleotide U downstream provides the lowest⁴⁰.

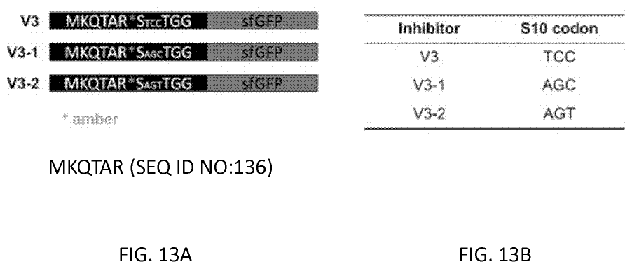

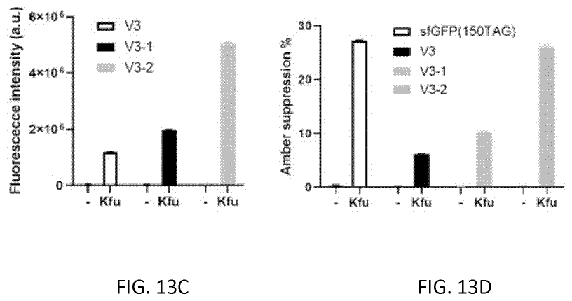

In the V3 inhibitor, the amber stop codon locates at the histone H3 K9 position. The codon following lysine 9 is TCC, which codes for a serine. Based on the context effect on amber suppression, nucleotide T (U on mRNA) is unfavorable for high-efficiency amber suppression⁴⁰. To address this problem, I replaced the TCC codon of serine with AGC or AGT (two synonymous codons of TCC), which also code for serine to generate inhibitors V3-1 and V3-2 (FIGS. 13a and b).

I then compared the protein expression and amber suppression efficiency of V3, V3-1, and V3-2. The results indicate that replacement of the unfavorable U downstream of the amber stop codon to A indeed enhanced protein expression level and amber suppression efficiency (FIGS. 13c and d). I also noted that the amber suppression efficiency of V3-2—only one nucleotide different from V3-1(AGC vs. AGT)—is 2.5 times that of V3-1, which further demonstrates the importance of context effect in amber suppression (FIG. 13d). I also compared the amber suppression efficiency of V3-2 with sfGFP150TAG and determined that the amber suppression efficiency of V3-2 is similar to sfGFP150TAG (FIG. 13d). Since V3-2 has exactly the same amino acid sequences with V3 and only differs in the nucleotide sequence, V3 will be used hereafter to denote the protein inhibitor.

### 6.2.3 In Vitro Characterization of V3 Inhibitor

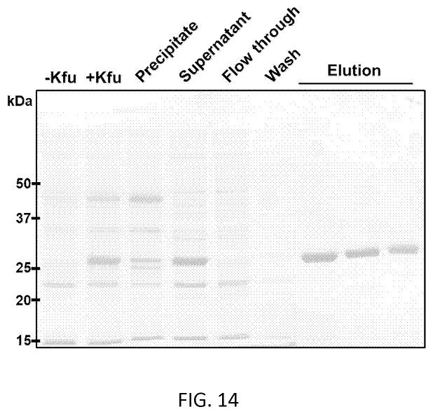

Following the optimization of V3 inhibitor expression, I expressed and purified V3 protein from DH10B E. coli cells. V3 was expressed in the presence of KfuRS/PyltRNA pair and 1 mM Kfu and purified using a Ni-NTA column (FIG. 14).

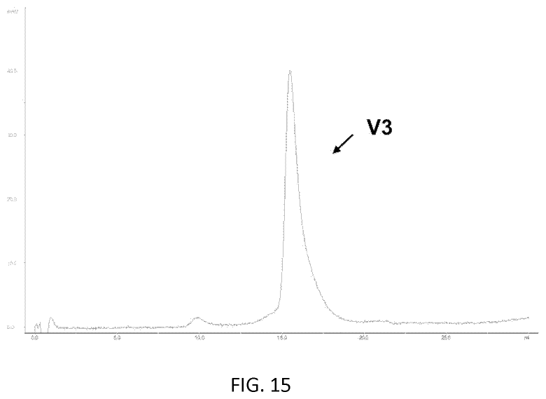

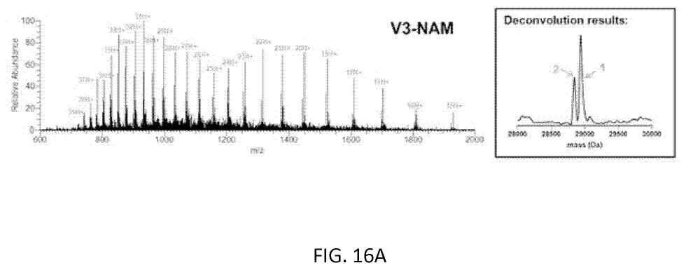

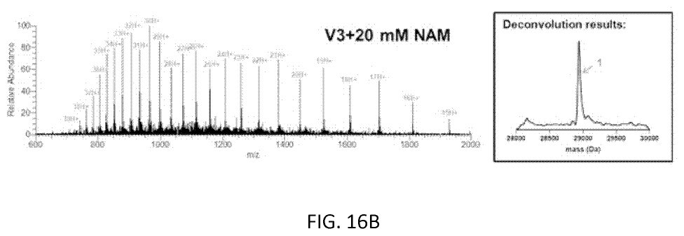

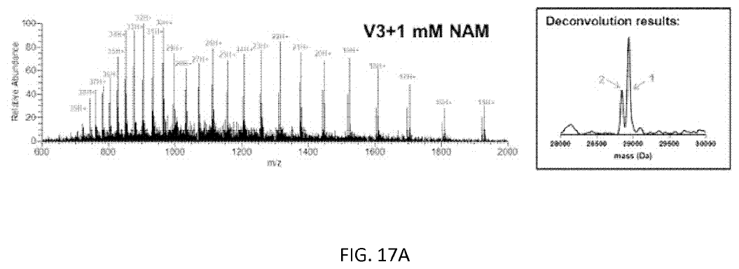

The purity and homogenity of purified V3 protein was then assessed by gel filtration (FIG. 15). Results showed that V3 is pure and exists as monomer. The identity of V3 protein was assessed by ESI-MS, and deconvolution results showed that—besides the major peak corresponding to V3 protein—there was a minor peak whose mass was equal to 2-furancarbonyl deacylated V3 protein (FIG. 16a).

Since nicotinamide (NAM) is a known inhibitor of the sirtuin family of enzymes, I expressed V3 in the presence of 20 mM NAM. The ESI-MS analysis of V3 produced in the presence of 20 mM NAM exhibited a single peak corresponding to the V3 protein, with no peak observed for 2-furancarbonyl deacylated protein (FIG. 16b). Thus, I concluded that CobB would remove 2-furancarbonyl from Kfu incorporated into V3 and that NAM completely inhibits the deacylation activity of CobB in E. coli.

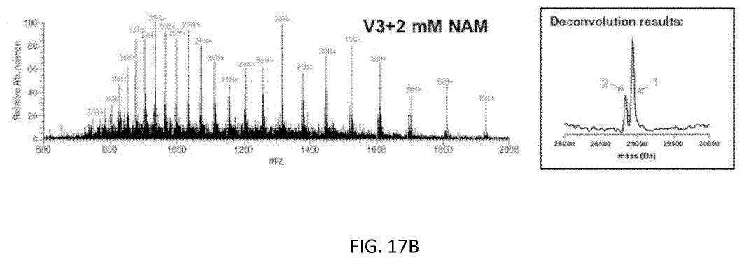

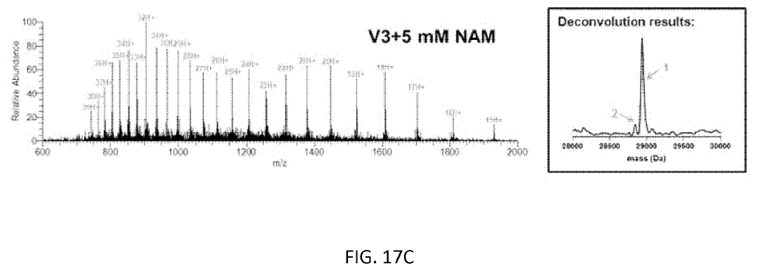

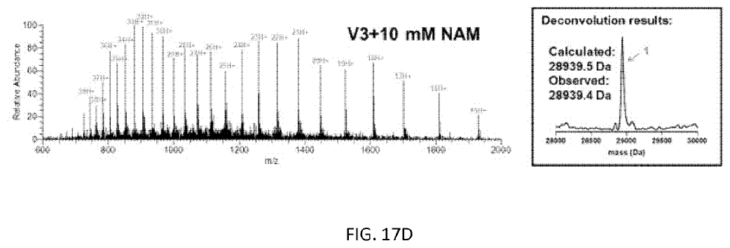

However, the V3 protein expression level was severely attenuated in the presence of 20 mM NAM (data not shown). Therefore, I attempted several lower NAM concentrations and assessed the purified V3 protein using ESI-MS. The results indicate that 10 mM NAM was sufficient to inhibit the deacylation activity of CobB (FIG. 17) while having a negligible impact on V3 protein expression level (data not shown). Thus, 10 mM NAM was added to the culture medium for the subsequent expression of V3 in E. coli.

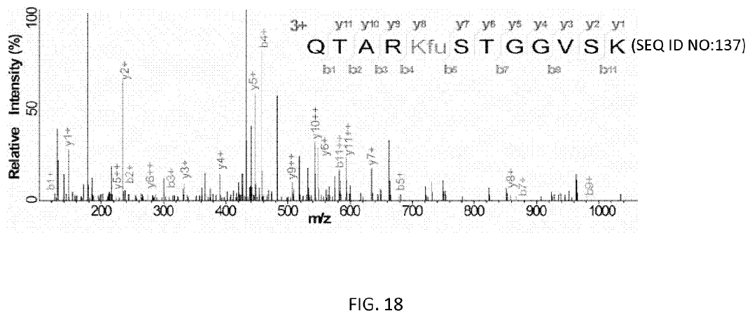

To demonstrate that the incorporation of Kfu into V3 protein occurs in a site-specific manner, purified V3 protein was digested with Lys-C, and the resulting peptides were analyzed by HPLC-MS/MS. The MS/MS spectrum of a peptide of V3 clearly demonstrates that Kfu was site-specifically incorporated at the V3 amber position (FIG. 18).

Next, I tested whether V3 protein could bind to the YEATS domains and act as a YEATS domain inhibitor. Isothermal titration calorimetry (ITC) was performed to determine the dissociation constant (Kd) between V3 and all four YEATS domain-containing proteins in humans (i.e. AF9, ENL, Gas41, and YEATS2).

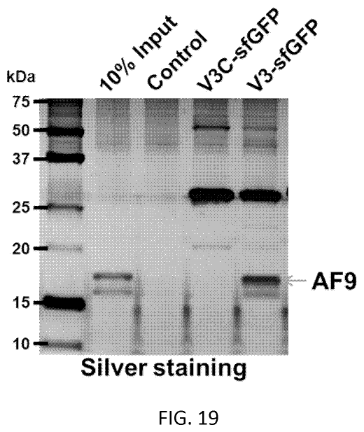

ITC was first performed with V3 protein and the AF9 YEATS domain. The obtained K_{d} (2.35 μM) was in strong agreement with a previously reported result for the peptide-based inhibitor (inhibitor 4.7, K_{d}=3.3 μM, data from my colleague LiXin's thesis) (FIG. 19a). To demonstrate whether the binding of V3 to AF9 YEATS domain is indeed dependent on Kfu incorporated at the H3 K9 position of V3, I expressed and purified V3C protein (contains a normal lysine at the H3 K9 site) and performed ITC with V3C protein and the AF9 YEATS domain. The results indicate no detectable binding between V3C and the AF9 YEATS domain (FIG. 19b).

Then, I performed ITC with V3 protein and the other three YEATS domain-containing proteins in humans to evaluate the selectivity of V3. The results indicate V3 protein has no detectable binding with ENL, Gas41, and YEATS2 proteins (FIG. 19c, d, e).

### 6.2.4 Optimization of the Expression of V3 Inhibitor in Mammalian Cells

Next, I sought to express V3 in mammalian cells. The PylRS/PyltRNA pair from archaea is orthogonal with the translational machinery in mammalian cells; therefore, the KfuRS/PyltRNA pair evolved from E. coli can also be used in mammalian cells. However, the protein yield of the PylRS/PyltRNA pair in mammalian cells is relatively low, which may cause a low cellular concentration of target protein and hamper the utility of V3 as an inhibitor.

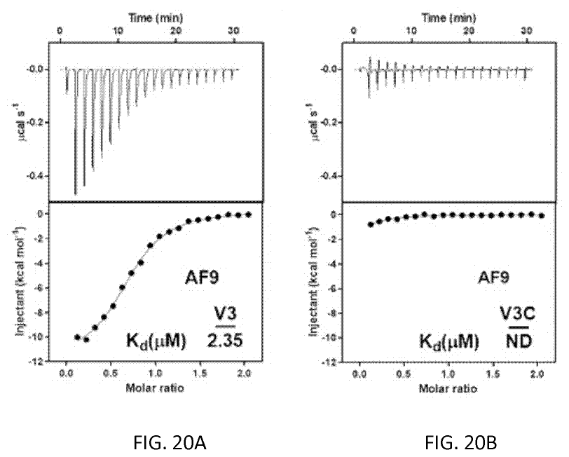

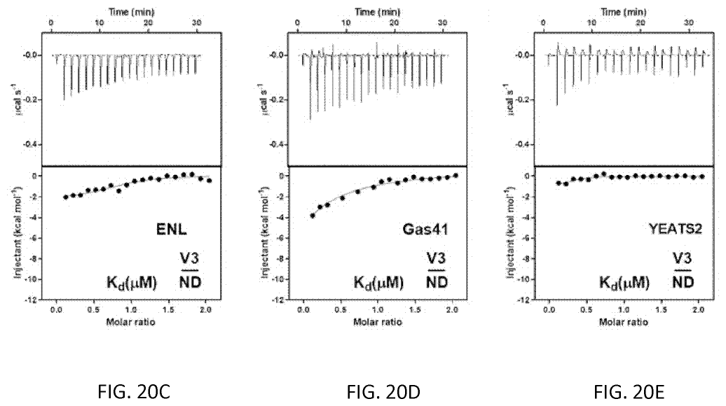

To address this issue, we constructed different plasmid constructs to express a PylRS/PyltRNA pair. As shown in FIG. 20a, construct a, b, and c code for the PylRS and PyltRNA genes. Construct a contained one copy of the PylT gene under the control of the U6 promoter and one copy of the MmKfuRS gene under the control of the CMV promoter. Construct b was the same as a, except for the PylRS gene, which was from Methanosarcina barkeri archaea (MbPylRS). Construct c contained a human codon-optimized hMbPylRS gene under the control of the CMV promoter and four copies of the PylT-M15 gene (containing A10G, U14G, U16G, U20C, U25C, and A52C, confer higher amber suppression in mammalian cells⁴⁶) under the control of the U6 promoter. Constructs 1, 2, and 3 were used for the expression of the target protein (i.e., V3) and contained additional copies of the PylT or PylT-M15 gene to increase PylT expression.

Different combinations of the constructs were co-transfected to HEK 293T cells. The expression of V3 from each pair of constructs was quantified by measuring the sfGFP fluorescence of cell lysates. The results indicate that the highest expression of V3 originated from constructs c+3 (FIG. 20b).

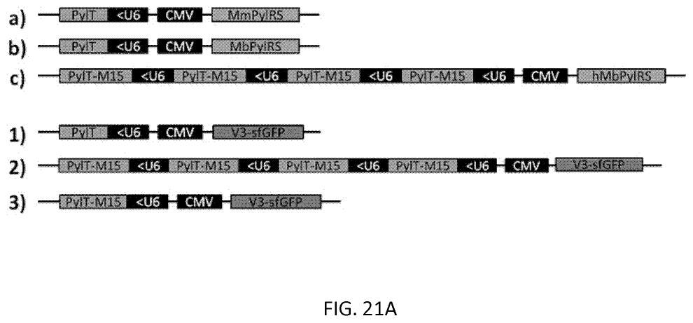

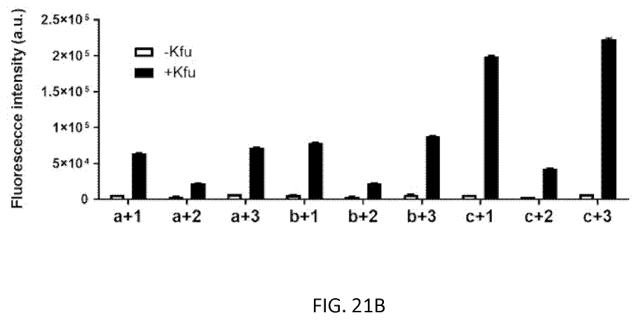

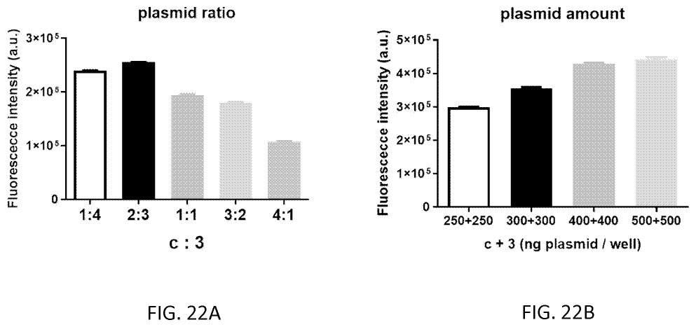

To further enhance the expression of V3, I attempted different ratios of construct c and construct 3 for transfection. To complete this, the total amount of c+3 plasmids was fixed at 500 ng/well (24-well plate). Then, different ratios of c and 3 were used to transfect HEK 293T cells cultured in a 24-well plate. Twenty-four hours after transfection, fluorescence from the cell lysates was measured using a plate reader. The results suggest that the transfection of cells with a plasmid ratio of c:3=2:3 gave the highest V3 expression (FIG. 21a). I also attempted to increase the total amount of plasmids used for transfection with the ratio of c:3 fixed to 1:1 and transfect cells with varying amounts of plasmids. Fluorescence from the cell lysates was measured 24 hours after transfection. The results indicate that V3 expression reached a plateau when 800 ng of plasmids were used for transfection (FIG. 21b). The expression of V3 at different time points was also examined, and the results indicate that the V3 expression level increased with time (FIG. 22a). I also calculated the amber suppression efficiency of V3 at different time points. The results showed that the amber suppression efficiency was as high as 42% at 24 hours, while at 48 hours and 72 hours, amber suppression efficiency remained at 30% (FIG. 22b).

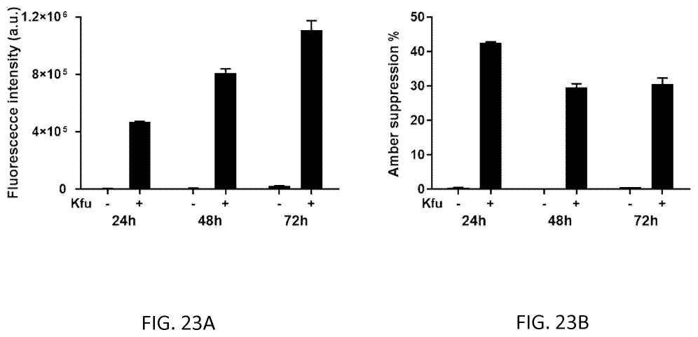

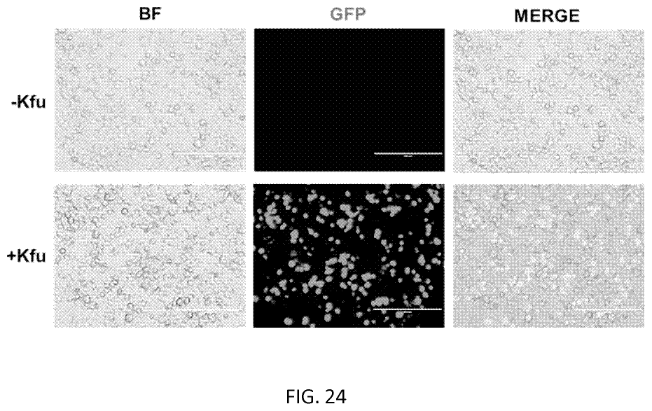

The expression of V3 in mammalian cells was further verified by fluorescence imaging. HEK 293T cells transfected with c and 3 and cultured with or without Kfu were examined under a fluorescence microscope. As expected, V3 could only express in the presence of Kfu (FIG. 23). The expression of V3 in HEK 293T cells was also verified by immunoblotting with an anti-GFP antibody (FIG. 24a).

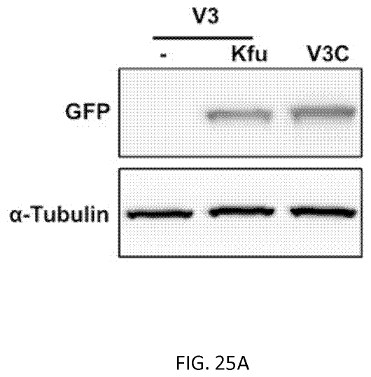

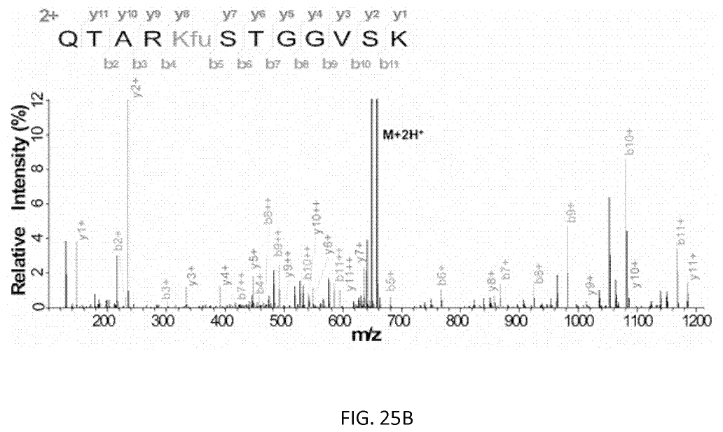

To verify that the incorporation of Kfu into V3 was in a site-specific-manner. V3 was expressed and purified from HEK 293T cells by Ni-NTA agarose. Then, V3 was in-gel digested by Lys-C, and the resulting peptides were analyzed by LC-MS/MS. The results indicate that Kfu was site-specifically installed at the K9 position of V3 (FIG. 24b). The effect of V3 expression on endogenous AF9 expression was also examined. The results show that the expression of V3 or V3C would not influence the expression of endogenous AF9 (FIG. 25).

### 6.2.5 Genetically Controlling the Subcellular Localization of V3 Inhibitor

Since all four YEATS domain-containing proteins in humans localize to the cell nucleus¹⁴, it is reasonable to restrict the localization of V3 to the nucleus, which may increase its nuclear concentration and enhance its inhibitory effects. The subcellular localization control of V3 is also a benefit of genetically encoded inhibitors, which could not be achieved by the peptide-based inhibitors.

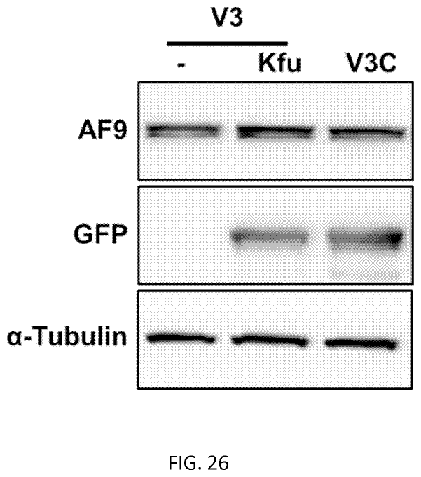

To achieve this, I fused the NLS from nucleoplasmin protein to the C-terminal of V3 (V3-NLS). To better demonstrate the ability to control the subcellular localization of V3 inhibitor genetically, I constructed another version of V3 with a nuclear export sequence (NES) fused to the C-terminal of V3 (V3-NES). Confocal images of cells expressing V3 with different subcellular localization sequences showed that, without any subcellular localization sequence, the V3 inhibitor mainly localized in the nucleus with a small amount in the cytosol. When NLS was added to the C-terminal of the V3 inhibitor, V3-NLS exclusively localized to the nucleus; in contrast, the majority of V3-NES localized to cytosol when NES was added (FIG. 26). In summary, the subcellular localization of the genetically encoded V3 inhibitor can be controlled, which may enhance its inhibitory effects in cells.

### 6.2.6 V3 Inhibitor Disrupts the Chromatin Binding of AF9 Protein

In one embodiment, fluorescence recovery after photobleaching (FRAP) may be used to analyze diffusion and binding of proteins in living cells. It has also been used to assess the cellular efficiency of bromodomain inhibitors in living cells.

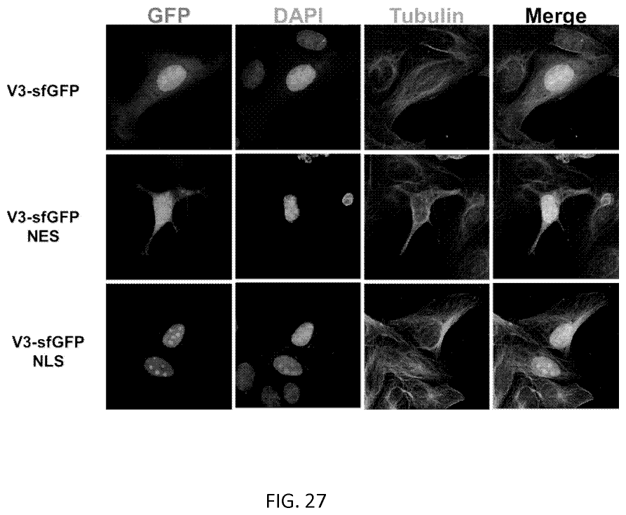

In the FRAP experiments, the protein of interest is genetically tagged with a fluorescent protein (e.g., GFP) and expressed inside the cell. Then a portion of the fluorescent-tagged protein are photobleached by an intense laser pulse, and the fluorescence recovery of that photobleached region, which is caused by the migration of fluorescent-tagged proteins back into the bleached area, is monitored over time (FIG. 27a). A fluorescence recovery curve can be generated by plotting the fluorescence intensity with time, and the half time (t_{1/2}), which is the time that the fluorescence intensity takes to recover to half, could also be calculated (FIG. 27b).

Take the chromatin associated bromodomain-containing proteins as an example, after photobleaching, the migration of unbleached fluorescently-tagged proteins back into the bleached region is retarded by the binding of bromodomain-containing proteins to chromatin and is thus slower compared to freely diffusible proteins.

Since AF9 protein was reported to associate with chromatin by its YEATS domain, we reasoned that the V3 inhibitor would reduce the binding of AF9 to chromatin and increase its mobility. To test this hypothesis, I fused sfGFP to the C-terminal of AF9 protein and replaced the C-terminal sfGFP of V3-NLS and V3C-NLS to a red fluorescence mCherry protein to avoid fluorescence overlap in FRAP experiments.

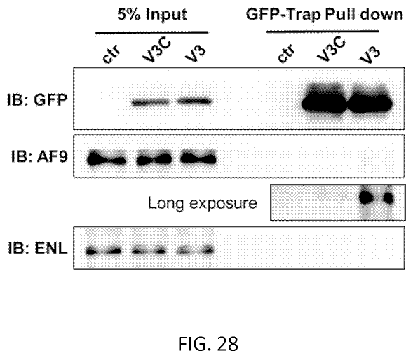

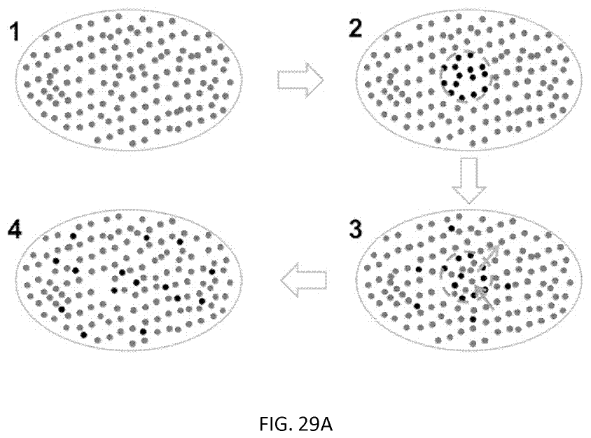

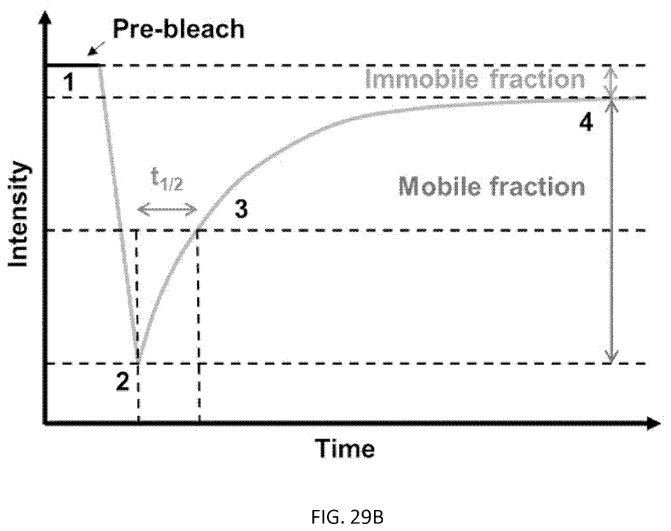

In the FRAP experiment, HEK 293T cells were transfected with AF9-sfGFP, and FRAP was performed 24 hours after transfection. The AF9-sfGFP fusion protein was exclusively localized to the nucleus (FIG. 29a). Photobleaching of a 4.5 μm² area of the nucleus resulted in a gradual recovery of >90% of the initial intensity, indicating that the majority of AF9-sfGFP protein was mobile, with a half recovery time (t_{1/2}) of 2.5±0.1 seconds (FIG. 28). If the cells were treated with the histone deacetylase (HDAC) inhibitor suberoylanilide hydroxamic acid (SAHA, 2.5 μM) for 18 hours before FRAP analysis—which was expected to increase the global acetylation level of chromatin—the half recovery time (t_{1/2}) of AF9-sfGFP would increase to 4.7±0.2 seconds (FIG. 28).

Notably, the final fluorescence (fluorescence at plateau) of SAHA-treated cells was only 75% of the initial intensity, and thus much lower than the untreated cells (75% vs. 90%) (FIG. 28a). A reasonable explanation for this is that the increased chromatin acetylation caused by SAHA treatment enhanced the chromatin binding of AF9-sfGFP, thereby reducing the mobile fraction pool of AF9-sfGFP (FIG. 27b). Together, these observations demonstrated the feasibility of FRAP to assess the chromatin binding state of AF9 protein.

Then, I performed AF9-sfGFP FRAP analysis with cells expressing either V3-NLS (mCherry) inhibitor or V3C-NLS (mCherry) inhibitors. I also generated a construct expressing AF9(F59A)-sfGFP protein bearing the F59A mutation on AF9, which abolished its binding to Kac and Kcr marks¹⁷, thus acting as a positive control. To generate a larger window for FRAP analysis, 2.5 μM SAHA was added to all of the groups. The results indicate that V3-NLS (mCherry) inhibitor significantly reduced the half recovery time (t_{1/2}=3.1±0.2 s) when compared to the no inhibitor control (t_{1/2}=4.7±0.2 s) and the V3C-NLS (mCherry) control (t_{1/2}=4.2±0.2 s) (FIG. 29). Cells expressing AF9(F59A)-sfGFP exhibited the fastest recovery, with a half recovery time of 2.3 seconds (t_{1/2}=2.3±0.1 s) (FIG. 29). In summary, the FARP analysis clearly demonstrates that the V3 inhibitor could bind to AF9 protein and reduce its binding to chromatin in living cells.

### 6.2.7 V3 Inhibitor Reduces the Genomic Localization of AF9 Protein on its Target Genes

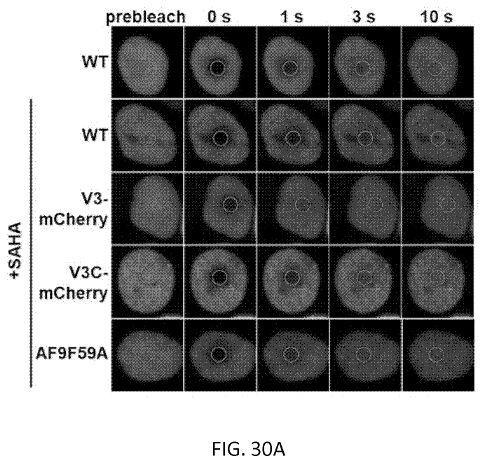

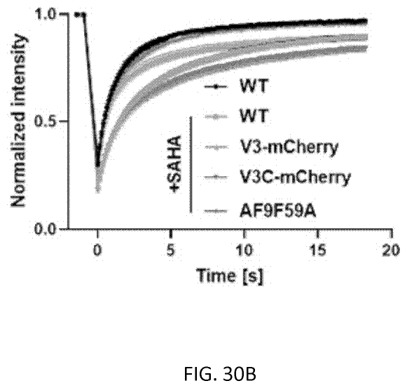

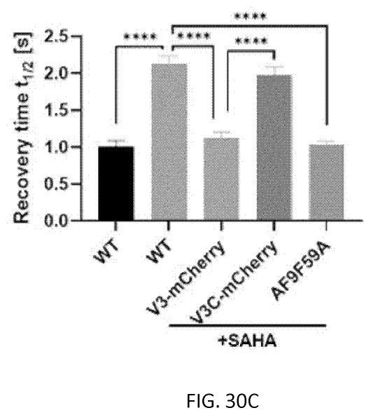

Next, I investigated whether the V3 inhibitor will inhibit the cellular function of AF9 protein. Given the fact that AF9—as a component of the super elongation complex (SEC)—positively regulates gene transcription in a YEATS domain-dependent manner^{12, 17}, I performed chromatin immunoprecipitation (ChIP) of the AF9 protein in HEK 293T cells expressing either V3-NLS or V3C-NLS and examined the genomic localization of AF9 protein on its target genes (MYC and PABPC1) by quantitative PCR (qPCR). The results indicate that the expression of V3 significantly reduced the occupancy of AF9 on MYC and PABPC1 (FIG. 30a). The ChIP of ENL was also performed to evaluate the selectivity of V3 in vivo. The results showed that the localization of ENL on its target gene, MYC, was not influenced by V3 expression (FIG. 30b).

### 6.2.8 Discussion

### 6.2.8.1 Double-Sieve Selection for the Directed Evolution of PylRS

Various strategies have been developed to evolve the active aminoacyl tRNA synthetase (aaRS) toward a uAA from the massive aaRS library. However, until now, the double-sieve selection strategy remains the most widely used strategy for aaRS evolution due to its simplicity and robustness.

In this chapter, I constructed a double-sieve selection system for the directed evolution of PylRS toward Kfu. A highly active KfuRS mutant was discovered from the selection, thereby demonstrating the utility of this system. The replacement of Kfu with Koxa in our genetically encoded YEATS may generate a stronger inhibitor. In one embodiment, provided herein is a method of screening the PylRS mutant specific for Koxa via this double-sieve selection strategy.

In certain embodiments, the double-sieve selection system could not only be used for the directed evolution of aaRS but for the directed evolution of other components in GCE (e.g., tRNA, elongation factor Tu (EF-Tu), and even ribosome).

### 6.2.8.2 Advantages of a Genetically Encoded Inhibitor

In one embodiment, provided herein is a strategy using genetically encoded ubiquitin variants (UbVs) to modulate enzymes in the ubiquitin system. UbVs were engineered by phage display to target different components in the ubiquitin system. Using this strategy, highly specific and potent UbVs that bind to deubiquitinating enzymes (DUBs), E3 ligases, E2 ligases, and ubiquitin-binding proteins may be generated. These UbVs provide valuable tools for the study of ubiquitin systems. In certain embodiment, the method may be used to generate UbVs targeting other cellular proteins (i.e., transcription factors, PTM readers) and the selected UbVs may be potent inhibitors for these proteins. The binding affinity of the UbVs with their target proteins ranged from several nanomolar to several hundred nanomolar.

In certain embodiment, provided herein is a method of engineering a genetically encoded protein inhibitor targeting the KEAP1-NRF2 interaction (K_{d}=300 μM) based on the fibronectin type III domain (FN3) scaffold to probe the biological functions of KEAP1-NRF2 interaction. Unlike the aforementioned genetically encoded inhibitors that execute their functions by binding to their target proteins, this inhibitor shuts down the protein synthesis of the cell by posttranslationally modifying the 28S rRNA of the ribosome. Cell-type-specific and temporal control of the inhibitor is achieved by the cell-type-specific and inducible promoters.

Genetically encoded inhibitors show several advantages over peptide-based inhibitors. As summarized in table 1, poor cell-membrane permeability and low stability toward cellular proteases are two common limitations of peptide-based inhibitors; however, such limitations are no longer a problem for genetically encoded inhibitors, which are directly expressed in the cell and well folded to protect themselves from intracellular protease cleavage. Peptide-based inhibitors have a smaller size and more conformational flexibility, which restricts its affinity and specificity toward target proteins. Genetically encoded inhibitors have a more extensive interaction interface, which enables higher affinity and specificity toward its targets⁶⁸. Subcellular localization and cell-type-specific control, which are infeasible for peptide-based inhibitors, can easily be achieved for genetically encoded inhibitors by fusing cellular localization sequences and using cell-type-specific or tissue-specific promoters.

TABLE 1
Comparison of peptide-based inhibitors
with genetically encoded inhibitors.

| | Peptide-based | Genetically encoded |
|---|---|---|
| | inhibitor | inhibitor |
| Cell permeability | Low | Expressed inside cell |
| Intracellular stability | Low | High |
| Affinity | Low to medium | Medium to very high |
| Specificity | Low to medium | Medium to very high |
| Subcellular localization | No | Yes |
| Cell-type-specific | No | Yes |

### 6.2.8.3 Outlook for Genetically Encoded YEATS Domain Inhibitor

Regarding the genetically encoded YEATS inhibitor described in this chapter, we first incorporated an uAA, Kfu, into the genetically encoded YEATS inhibitor. The 2-furancarbonyl group—which does not exist in natural amino acids—of the genetically encoded YEATS inhibitor forms stronger π-π-π stacking with its target protein AF9, and thus acts as a competitive inhibitor of AF9. The 20 genetically encoded amino acids only carry a limited number of functional groups, thereby limiting the diversity of genetically encoded inhibitors. Since uAAs carrying much more diverse functional groups could be incorporated into proteins^{34, 69-74} incorporating uAAs into the genetically encoded inhibitors should become a general strategy to expand the diversity of genetically encoded inhibitors.

Secondly, in vitro ITC data showed that the genetically encoded YEATS domain inhibitor V3 binds to the AF9 YEATS domain with a moderate binding affinity (K_{d}=2.35 μM). Moreover, the V3 inhibitor has an inert sfGFP scaffold that should not be involved in the interaction with AF9. To further enhance the binding affinity of V3 toward AF9, other protein scaffolds that introduce additional interaction to AF9 could be used. For example, Dot1L is known to interact with the C-terminal of AF9; therefore, inserting the AF9-interacting peptide sequences of Dot1L into the genetically encoded inhibitor may generate an inhibitor with a better affinity and specificity toward AF9.

Lastly, the incorporation of Kfu in mammalian cells in the present study is based on transient expression, which limits its application to cell lines with high transfection efficiency. Since transient expression leads to heterogeneous expression levels in mammalian cells, the ability of investigators to study the global cellular effects resulting from the heterogeneous expression of V3 inhibitor remains limited. An ideal method for incorporating Kfu in mammalian cells would express the KfuRS/PyltRNA pair and the genetically encoded inhibitor from an integrated locus, thereby facilitating the uniform expression of V3 for all cells in a clonal population.

In one embodiment, cells are transfected with vectors encoding one copy of PylRS under the control of the EF1 promoter with eight copies of PylT under the control of the U6 promoter as well as an amber-bearing fluorescent reporter. Cells showing high-level amber suppression were screened out using flow cytometry. Using this strategy, they constructed mouse embryonic stem cell and mouse embryonic fibroblast cell lines with stable amber suppression and investigated the effect of genetically encoded histone acetylation on gene transcription.

In one embodiment, cells are transfected by vector encoding one copy of PylRS under the control of CMV promoter with 18 copies of the PylT gene under the control of the U6 promoter. Cells with high amber suppression were screened out using flow cytometry.

Since YEATS domain-containing proteins are frequently implicated in cancers¹³, expression of the genetically encoded YEATS inhibitor in physiologically relevant cancer cell lines would be very helpful in studying the biological functions of YEATS-Kcr/Kac interaction. In one embodiment, the KfuRS/PylT pair is integrated and the genetically encoded YEATS domain inhibitor into the genome of certain cancer cell lines and generate a cell line with stable amber suppression to aid in future YEATS domain studies.

### 6.3 Apply the Genetically Encoded YEATS Domain Probe for YEATS Domain Recruitment

### 6.3.1 Introduction

In last section, we demonstrated the application of the genetically encoded YEATS domain probe for inhibiting AF9, which is one of the four human YEATS domain proteins. Administration of the genetically encode YEATS domain inhibitor to the cells disrupted the chromatin localization, as well as genomic localization of AF9 proteins. In this section, we further extended the application of the genetically encoded YEATS domain probe for targeting YEATS domain protein to a specific genomic locus. To this end, we first chose the lacO-lacR tethering system.

LacO-lacR tethering system is based on the lactose operon (lac operon), which is widely adopted by E. coli and many other enteric bacteria to control the expression of genes which are required the transport and metabolism of lactose⁷⁷. The lac operon has also been engineered to turn on recombinant protein expression in E. coli⁷⁸. The bacterial lac operon contains a handful of genes, a promoter sequence and an operator sequence (lacO), which is bind by a transcription repressor (lacR). LacR bind tightly with lcaO sequence, thus preventing the transcription of downstream genes⁷⁷. The tight and specific binding between lacR and lacO has been utilized for the development of lacO-lacR tethering system.

LacO-lacR tether system is initially designed for visualization of the dynamic of centromere over time in yeast. For this purpose, lacO arrays DNA sequences are integrated into a genomic region, which is close to a specific centromere. LacR is fused with a fluorescent protein (e.g., GFP), and the fusion protein is constitutively expressed inside the cells. When combined, the lacR-GFP fusion protein would specifically bind with lacO arrays and light up only the genome locus bearing lacO arrays sequences.

### 6.3.2 Recruit YEATS Domain Protein to a Specific Genome Locus by the Genetically Encoded YEATS Domain Probe

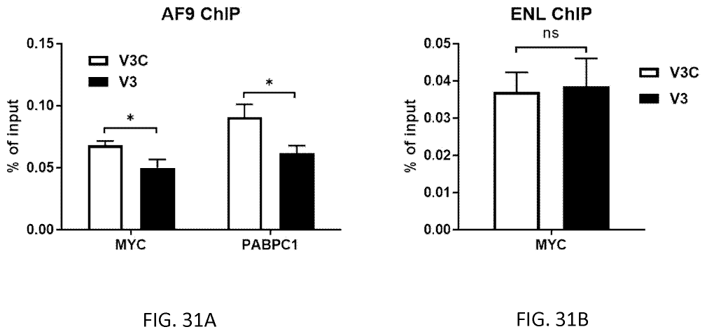

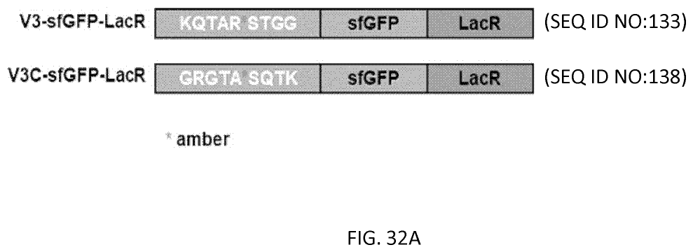

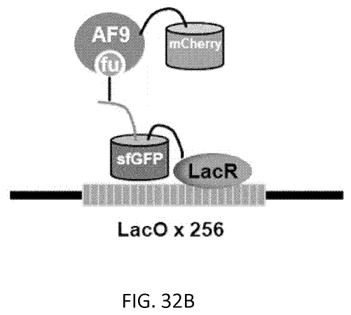

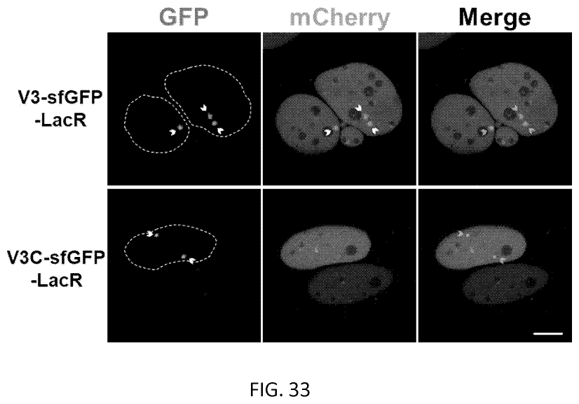

To extend the application of the genetically encoded YEATS domain probe for YEATS domain proteins recruitment, we planned to utilize the lacR-lacO system to showcase. As shown in FIG. 31a the genetically encode YEATS domain probe is fused with sfGFP protein at N-terminal, and the lacR repressor is fused with sfGFP at C-terminal. When this fusion protein is expressed in cell which bearing lacO arrays, the interaction between lacR and lacO would recruit the fusion protein to lacO arrays and result in a brightly sfGFP concentrated dot (FIG. 31b, FIG. 32). To see whether the genetically encoded YEATS probe would recruit YEATS domain proteins to the lacO arrays, we also expressed mCherry tagged AF9 protein in the cell, if the probe could recruit AF9 protein, we expected to observe a mCherry concentrated dot at lacO arrays (FIG. 31b).

Then we constructed the plasmids for expression of V3-sfGFP-lacR and AF9-mCherry, in addition, we also constructed a plasmid for expression of a control probe which have a scramble V3 sequence (V3C-sfGFP-lacR). A U2OS cell line with 256 copies of lacO arrays (U2OS-lacO) stably integrated into two chromosomal locations was purchased from kerafast company.

As we have expected, when KfuRS, V3-sfGFP-lacR and AF9-mCherry were co-expressed in U2OS-lacO cell line, we could observe the mCherry concentrated dot which is co-localized with sfGFP concentrated dot, suggesting the recruitment of AF9-mCherry to the lacO arrays (FIG. 32). In contrast, we didn't observe mCherry concentrated dot that is colocalized with lacO arrays in cells expressing V3C-sfGFP-lacR (FIG. 32), indicating the recruitment of AF9-mCherry protein is dependent on the genetically encoded YEATS domain probe. Together, these data demonstrated the ability of the genetically encoded YEATS domain probe for YEATS domain protein recruitment.

### 6.3.3 Discussion

Our genetically encoded YEATS domain probe enables modulating YEATS domain proteins, thus, aid to investigate the biological role of YEATS domain proteins. In this section, we showed that, besides acting as a YEATS domain inhibitor when fused to a non-chromatin protein, the genetically encoded YEATS domain probe could also recruit YEATS domain proteins to a specific genome locus by fusing to a DNA binding protein.

In the presented example, we used lacR protein that specifically bind to lacO DNA sequence. Other DNA binding proteins (e.g. transcription factors) could also be used. When the genetically encoded YEATS domain probe is fused with a transcription factor, the binding to the transcription factor to its target genes could also recruit YEATS domain proteins to the same genes, enabling elucidation of the effect of YEATS domain proteins on gene transcription at high resolution.

In one embodiment, the genetically encoded YEATS domain probe may be fused to dCas9 protein (a mutation of Cas9 without endonuclease activity), which could target any gene within the genome with guidance of small guide RNA (sgRNA), thereby recruiting YEATS domain proteins to a certain gene. This makes it possible to examine the effects of YEATS domain proteins on certain gene without the need to manipulate the YEATS domain proteins globally.

Altogether, in this section, we applied the genetically encoded YEATS domain probe for YEATS domain proteins recruitment, which further demonstrated the potential of the probe as a tool in modulating YEATS domain proteins.

### 6.4 Apply the Genetically Encoded YEATS Domain Probe for Cell-Based Screen of YEATS Domain Inhibitors

6.4.1 YEATS domain is a family of epigenetic reader module that specifically recognize histone acylations, such as acetylation and crotonylation. By reading histone acylations, YEATS domain participates in lots of chromatin-associated biological processes and regulate gene transcription. Given their functional importance, dysfunction of YEATS domain proteins is often linked to human diseases, such as cancer. YEATS domain is, therefore, emerging as a promising drug target for cancer therapy.

Chemical probes for YEATS domain are useful tools to interrogate the functions of YEATS domains in epigenetic regulation, as well as offer potential therapeutic agents targeting the YEATS domain. In the pursuit of optimized chemical tools for the YEATS domain, medium to high-throughput screening (HTS)-compatible biochemical assays are easily accessed, but cell-based assays capable of supporting large-scale screening and validation of the probes are lacking. FRAP assay has been widely used to assess the cellular efficacy of bromodomain inhibitors in living cells, and recently, has also been applied to the evaluation of YEATS domain inhibitors^{50, 83}. However, due to the large cell-to-cell variations, this assay requires the analysis of significant number of cells (usually between 20 to 50) in each condition. Moreover, FRAP assay has a relative narrow dynamic range, especially for YEATS domain proteins which only transiently bind with chromatin and exhibiting very high mobility. These limitations of FRAP assay have restricted its scale to low throughput.

The cellular thermal shift assay (CETSA) is another well-established and facile technique for assaying inhibitor target engagement via ligand-induced thermal stabilization. While the use of immunoblotting for final readout for CETSA inherently limits the scale at which it can be implemented. Some groups have combined CETSA with NanoLuc technology to simplify the final detection and make it compatible with HTS assays, however the signal to noise ratio of these assays is still low (usually 2-3 folds of background luciferase signal), do not support the robust evaluation and comparison of different inhibitors in cells⁸⁴.

More recently, a NanoLuc-based complementary assay (NanoBiT) was also reported to evaluate the activity of YEATS domain inhibitors in cells. In the NanoBiT assay, the large fragment of NanoLuc (LgBiT) was fused with GAS41 protein and the small fragment of NanoLuc (SmBiT) was linked to histone H3.3. The co-expression of both proteins resulted in luciferase signal reflecting the interaction of GAS41 with in situ acetylated histone H3.3, disruption of the interaction by inhibitors will, therefore, reduce the luciferase signal. However, in situ acetylated histone proteins only account for a small population of total histone proteins, that was, only a small fraction of expressed SmBiT-H3.3 proteins was modified, contributing to the formation of intact NanoLuc and generating of luciferase signal. So, not surprisingly, the luciferase signal of this assay was only 2 folds over background, limited its broad applications.

Given the drawbacks of current methods for cell-based evaluation of YEATS domain inhibitors, a new technology was on demand. We thought that our genetically encoded YEATS domain probe could be used to develop a robust technology for this purpose.

### 6.4.2 A Luciferase-Based Two-Hybrid Assay for In Vivo YEATS Domain Inhibitor Evaluation and Screening

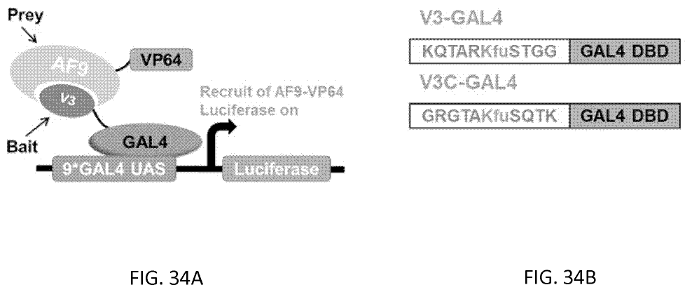

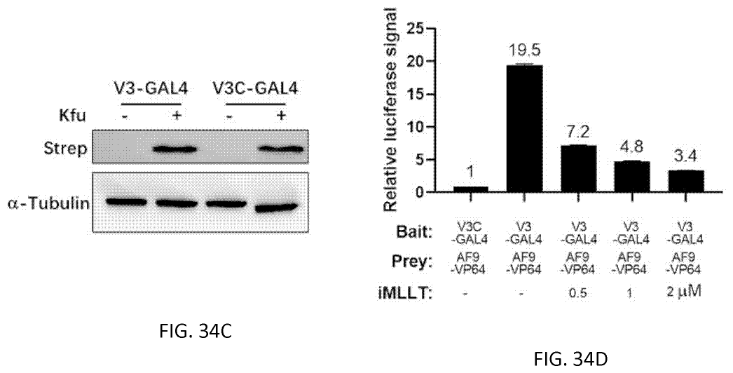

To solve the problems in current cell-based methods, as well as develop a robust cell-based technology for the in vivo screening and validation of YEATS domain inhibitors. We sought out to take advantage of the well-developed luciferase-based two-hybrid assay. In the two-hybrid assay, the genetically encoded YEATS domain probe was fused with a GAL4 DNA binding domain and served as ‘bait’, while a transcription activation domain (VP64) was linked to AF9 protein and acted as ‘prey’ (FIG. 34a, b, c). When co-express ‘bait’ and ‘prey’ proteins along with a luciferase gene under the control of 9 copies of GAL4 upstream activation DNA sequence (UAS), the ‘bait’ will bind with the ‘prey’ protein and form a protein complex (V3-GAL4/AF9-VP64) on the UAS of luciferase gene, activating the transcription of luciferase gene (FIG. 34a). The activation level of luciferase is around 20 folds, much higher than previously reported luciferase-based methods (usually ˜2 folds).

If a AF9 YEATS domain inhibitor, iMLLT⁸³, which disrupting the ‘bait’ and ‘prey’ interaction, was added to the cells, the formation of V3-GAL4/AF9-VP64 complex will be disrupted, and the transcription of luciferase was, thus, repressed upon inhibitor treatment (FIG. 34d). As shown in FIG. 34d, the luciferase signal was inversely proportional to the inhibitor concentration, demonstrating the ability this luciferase-based two-hybrid assay to evaluate the activity of different YEATS domain inhibitors in cells.

### 6.5 Experimental Methods

### 6.5.1 Plasmid Construction

pEVOL-KfuRS was constructed based on pEVOL-pAzF (from Addgene). MjtRNA_{CUA} gene in pEVOL-pAzF was first replaced by PylT gene. ProK promoter-PylT-proK terminator fragment was generated by multiple rounds of PCR. The first round of PCR was performed with primers TGF179 and TGF180 using pRep-PylT as the template. The products were used as the template for the next round of PCR. After four rounds of PCR with primers TGF181, TGF182, TGF183, and TGF184, ProK promoter-PylT-proK terminator fragment was generated with ApaLI and SphI sites at two ends. Then an intermediate vector pEVOL-PylT plasmid was generated by inserting ProK promoter-PylT-proK terminator fragment into pEVOL-pAzF. Two copies of KfuRS gene were successively inserted into pEVOL-PylT. The first copy of KfuRS gene was subcloned from pBK-KfuRS to pEVOL-PylT by double digestion (NdeI and PstI) and ligation. The second copy of KfuRS was amplified by PCR with primers TGF185 and TGF186 from pBK-KfuRS vector creating SpeI and SalI sites. Vector fragment was amplified by PCR with primers TGF193 and TGF194 from pEVOL-PylT (containing one copy KfuRS) creating SpeI and SalI sites. Then two fragments were joined by double digestion and ligation to generate pEVOL-KfuRS.

pBad-V1 and pBad-V1C were constructed based on pBad-sfGFP vector by adding NLS-GGGG-H3(4-13) fragment to the N-terminal of sfGFP. NLS-GGGG-H3(4-13, K9TAG) fragment of pBad-V1 was generated by PCR with primers TGF319, TGF320, and TGF321, NLS-GGGG-H3(4-13) fragment of pBad-V1C was generated by PCR with primers TGF319, TGF320, and TGF322. Vector fragment was generated by PCR with primers TGF317 and TGF318 from pBad-sfGFP. Insert and vector fragments were jointed by basic seamless cloning kit (TransGen).

pBad-V2C was constructed by deleting the H3(4-13) sequence from pBad-V1C using PCR-based mutagenesis with primers TGF339 and TGF340. pBad-V3C was constructed by adding the H3(4-13) sequence to pBad-sfGFP using PCR-based mutagenesis with primers TGF342 and TGF343.

pBad-V3 was constructed by adding the H3(4-13, K9TAG) sequence to pBad-sfGFP using PCR-based mutagenesis with primers TGF341 and TGF342. pBad-V3-1 and pBad-V3-2 were constructed the same as pBad-V3 except that using primers TGF342 and TGF344 for pBad-V3-1 and primers TGF342 and TGF345 for pBad-V3-2.

pCMV-MmKfuRS was modified from pCMV-MmPylRS-AF (from Addgene). PCR-based mutagenesis was used to introduce the point mutations with primers TGF348 and TGF349. pCMV-MbKfuRS was modified from pCMV-MbPylRS-DiZPK (from Addgene) using PCR-based mutagenesis with primers TGF329 and universal BGH-rev. pNEU-hMbKfuRS-4×PylTM15 was modified from pNEU-hMbPylRS-4×PylTM15 (from Addgene) using PCR-based mutagenesis with primers TGF346 and TGF347.

pCMV-V3-NLS was constructed by inserting V3-NLS into pCMV vector using basic seamless cloning kit (TransGen). V3-NLS fragment was amplified from pUC57-V3-NLS (synthesized by BGI, contain human codon-optimized sfGFP) by PCR with primers TGF356 and TGF357. Vector fragment was PCR amplified from pCMV vector with primer TGF354 and TGF355. pNEU-V3-NLS-4×PylTM15 was constructed by inserting V3-NLS fragment into pNEU vector using basic seamless cloning kit (TransGen). V3-NLS fragment was amplified from pUC57-V3-NLS by PCR with primers TGF352 and TGF353. Vector fragment was PCR amplified from pCMV vector with primer TGF350 and TGF351. pCMV-V3-NLS-PylTM15 was constructed from pCMV-V3-NLS by PCR-based mutagenesis with primers TGF387 and TGF388. pCMV-V3C-NLS-PylTM15 was constructed from pCMV-V3-NLS-PylTM15 by PCR-based mutagenesis with primers TGF379 and TGF380. pCMV-V3-PylTM15 was constructed from pCMV-V3-NLS-PylTM15 by PCR-based mutagenesis with primers TGF396 and TGF397. pCMV-V3-NES-PylTM15 was constructed from pCMV-V3-NLS-PylTM15 by PCR-based mutagenesis with primers TGF389 and TGF390.

pCMV-V3-mCherry-NLS-PylTM15 was constructed from pCMV-V3-NLS-PylTM15 by basic seamless cloning kit (TransGen). Insert fragment, mCherry, was amplified from pMaRSC (Addgene) with primers TGF434 and TGF435, vector fragment was amplified from pCMV-V3-NLS-PylTM15 with primers TGF431 and TGF432. pCMV-V3C-mCherry-NLS-PylTM15 was constructed the same as pCMV-V3-mCherry-NLS-PylTM15 except that vector fragment was amplified from pCMV-V3C-NLS-PylTM15 with primers TGF432 and TGF433.

To construct pcDNA3.1-AF9-sfGFP, basic seamless cloning was used. The vector fragment was amplified from pMaRSC (with pcDNA3.1 backbone) with primers TGF375 and TGF430. The first insert fragment, AF9, was amplified from cDNA with primers TGF377 and TGF429, the second fragment, sfGFP, was amplified from pCMV-V3-NLS with primers TGF427 and TGF428. Then pcDNA3.1-AF9-sfGFP plasmid was formed by simultaneously fusing three fragments by basic seamless cloning. pcDNA3.1-AF9F59A-sfGFP was constructed from pcDNA3.1-AF9-sfGFP by PCR-based mutagenesis with primers TGF425 and TGF426.

### 6.5.2 PylRS Library Construction

The degenerate primers used for library construction were ordered from IDT. All the PCR for library construction was performed using the proofreading high fidelity DNA polymerase from Vazyme.

Randomization mutations at Y271, L274, and C313 were introduced by a PCR-based method. The first stage PCR consists of three individual reactions, 1) primers TGF105&TGF106 to introduce randomization mutations to Y271 and L274 sites, 2) primers TGF107&TGF157 to introduce randomization mutations to C313, 3) primer TGF109&TGF110 to amplify the C-terminal fragment of PylRS. pBK-PylRS (Y349F) plasmid was used as the template for the PCR. The PCR products were then digested with DpnI to remove the template plasmid and purified by gel or column.

The second stage overlap extension PCR was performed by mixing equal molar of each fragment and amplified for 15 cycles. Then end primers (TGF105&TGF110) were added to amplify the full-length PylRS from the mixture. The full-length PylRS bearing randomization mutations at desired positions were purified by gel, digested with NdeI and PstI, and ligated into pre-digested pBK vector with T₄ ligase (NEB). The ligation products were used to transform DH10B chemical competent cells (Invitrogen), after recover for 1 hour in SOC medium, E. coli culture was added to 0.5 L LB medium containing 50 μg/mL kanamycin and incubated at 37° C. for 12 h, a small portion of culture were also spread on LB agar plate containing 50 μg/mL kanamycin. Plasmid encoding PylRS library was extracted from the culture, size of the library was calculate based on the number of colonies on LB agar plate, quality of the library was checked by sequencing randomly chosen colonies from the LB agar plate.

### 6.5.3 Double Sieve Selection

In positive selection, E. coli DH10B harboring the positive selection plasmid pREP-PylT were transformed with the plasmid encoding PylRS library, plated on LB agar plate containing 50 μg/mL kanamycin and 10 μg/mL tetracycline (LB-KT), yielding 10 folds of the library size colonies. Colonies were scraped from the plate and incubated (37° C., 250 rpm) in 100 mL LB-KT to saturate. This culture was diluted 1:50 into fresh LB-KT plus 1 mM Kfu; at O.D 600 nm about 0.5, this culture was diluted (make sure at least 10 fold of library size cells were plated) and plated on LB plate containing 50 μg/mL chloramphenicol and 1 mM Kfu. After 48 h incubation at 37° C., cells on the plate were scraped, cultured in 50 mL LB-KT to saturate. The plasmid was extracted. pBK plasmid encoding PylRS library was separated from the positive selection plasmid by gel purification.

In negative selection, 50 ng of the pBK plasmid after the positive selection was used to transform DH10B cell harboring pAC-Bar plasmid; cells were plated on LB plate containing 50 μg/mL kanamycin and 100 μg/mL ampicillin (LB-KA) and 0.2% arabinose, incubated at 37° C. for overnight.

Cells passed negative selection were combined, cultured in 50 mL LB-KA to saturate, the plasmid was extracted. pBK plasmid encoding PylRS library was separated from the negative selection plasmid by gel purification. 50 ng pBK plasmid after the negative selection was used to transform DH10B cell harboring pAC-sfGFP150TAG; the cells were plated on LB-KT plate plus 0.2% arabinose and 1 mM Kfu and incubated at 37° C. for 1 day and RT for another 1 or 2 days to promote sfGFP expression. Then the visible green colonies (under 365 nm ultraviolet light) were picked and sent for sequencing.

### 6.5.4 SfGFP Fluorescence Assay in E. coli

Plasmids encoding PylRS/PyltRNA pair and fluorescence reporter (i.e. sfGFP150TAG, V3) were co-transformed to DH10B cells and plated on LB agar plate supplement with corresponding antibiotics. Colonies were inoculated to LB supplement with corresponding antibiotics and cultured at 37° C. for overnight. Then the overnight culture was 1:50 diluted in 100 NL fresh LB medium in 96 well plate and incubated with shaking (37° C., 250 rpm), 1 mM Kfu and 0.2% arabinose were added when O.D. 600 reached 0.4, the culture was continue incubated (37° C., 250 rpm) for 20 h. For sfGFP fluorescence measurement, the culture was 10-fold diluted with PBS; sfGFP fluorescence from the cell suspension was measured by plate reader (PerkinElmer) with excitation 485 nm and emission 535 nm.

### 6.5.5 SfGFP150Kfu Expression and Purification

DH10B cells harboring pEVOL-KfuRS and pBad-sfGFP150TAG plasmid were cultured in LB plus corresponding antibiotics. Protein expression was induced by adding 0.2% arabinose and 1 mM Kfu when O.D. 600 reached 0.4. After expression at 37° C. for 20 h, cells were harvested and washed with PBS. Protein was released from the cells by sonication in lysis buffer (50 mM HEPES pH 7.5, 500 mM NaCl, 10 mM Imidazole, 5% glycerol, freshly added 1 mM PMSF, protease inhibitor cocktail). The clear supernatant was separated from the precipitate by centrifugation (30 min, 25000 g, 4° C.), Ni-NTA agarose beads (Invitrogen) were added to the clear supernatant and incubated with agitation for 1 h at 4° C. The mixture was poured into a column (Biorad) and washed with 20 bed volumes of wash buffer (50 mM HEPES pH 7.5, 500 mM NaCl, 30 mM Imidazole, 5% glycerol). Proteins were eluted from the beads with 5 bed volumes of elution buffer (50 mM HEPES pH 7.5, 500 mM NaCl, 250 mM Imidazole, 5% glycerol) and dialyzed to storage buffer (50 mM HEPES pH 7.5, 150 mM NaCl, 5% glycerol). The purified proteins were snap freezed by liquid nitrogen and stored in −80° C. fridge. The quality of purified proteins was analyzed by 12% SDS-PAGE and gel-filtration.

### 6.5.6 V3 and V3C Expression and Purification in E. coli

DH10B cells harboring pEVOL-KfuRS and pBad-V3-2 plasmid were cultured in LB plus corresponding antibiotics. Protein expression was induced by adding 0.2% arabinose, 10 mM NAM, and 1 mM Kfu when O.D. 600 reached 0.4. After expression at 37° C. for 20 h, cells were harvested and washed with PBS. Protein purification was the same as sfGFP150Kfu except that 10 mM NAM was added to lysis buffer to prevent CobB deacylation. V3C was expressed and purified the same as V3.

### 6.5.7 Evaluation of Amber Suppression Efficiency in Mammalian Cells

HEK293T cells were cultured in complete growth medium at 37° C. in a 5% C02 atmosphere. The day before transfection seeded HEK293T cells in 24-well plates at a density of 1-1.5×10⁵ cells per well in 0.5 mL complete growth medium and let them reach 60-80% confluency at the time of transfection. 1 h prior to transfection, 1 mM Kfu was added to corresponding wells. Cells were transfected by lipofectamine 3000 reagent (Thermo) following the manufacture's instruction. 250 ng of each plasmid (plasmid encoding KfuRS/PyltRNA pair and plasmid encoding inhibitor) were used per well. 48 h after transfection, the medium was removed, and cells were lysed by adding 100 μL of SDS lysis buffer (20 mM Tris-HCl pH 8.0, 150 mM NaCl, 1.5 mM MgCl₂, 1% SDS, add Benzonase nuclease (Thermo) before use) to the wells. After 15 min at RT with gentle shaking, the lysate was clarified by centrifugation (20,000 g, 10 min, RT), the concentration of the clear lysate was measured by BCA kit (Thermo) and adjusted to 1 mg/mL before fluorescence measurement. SfGFP fluorescence of the lysate was measured by plate reader (PerkinElmer) with excitation 485 nm and emission 535 nm.

### 6.5.8 FRAP

HEK 293T cells were seeded in 3.5 cm confocal dish at a density of 4-6×10⁵ cells per well in 2 mL complete growth medium and let them reach 60-80% confluency at the time of transfection. 1 mM Kfu was added to corresponding wells 1 hour before transfection. Cells were transfected with pcDNA3.1-AF9-sfGFP or pcDNA3.1-AF9(F59A)-sfGFP plasmids (1 μg/well) and plasmids required for the expression of genetically encoded inhibitor (pNEU-KfuRS 800 ng/well, pCMV-V3-mCherry-NLS-PylTM15, 1.2 μg/well) or control inhibitor (pCMV-V3C-mCherry-NLS-PylTM15, 300 ng/well) by lipofectamine 3000 reagent (Thermo) following manufacture's instruction. 2.5 μM SAHA was added 6 hours after transfection, and FRAP was conducted 24 hours after the transfection. Cells were washed once with phenol red-free medium 1 hour before the FRAP experiment and cultured in phenol red-free medium during the whole experiment. The FRAP was performed with Carl Zeiss LSM710 NLO confocal microscope using a 40× oil-immersion objective. An argon laser (488 nm) was used for photobleaching and fluorescence imaging of sfGFP. The detector was set to detect fluorescence between 500 and 550 nm. For V3 or V3C group, only cells expressing both sfGFP and mCherry were chosen for FRAP. A 4.5 μm² circular region of the selected cell nucleus was bleached after five prescans, by 100% laser power (488 nm), and 20 iterations. Fluorescence recovery of sfGFP was monitored by a time series imaging. The images were acquired with a zoom factor of 14, frame size of 512 pixels×512 pixels, line-stepping of 1, and bidirectional scanning, which had a time interval of approximately 0.3 seconds.

The average fluorescence intensity was measured for three regions at each imaging time point, a bleached region (F_{bl}), a non-bleached reference region (F_{ref}) to correct for the FRAP curve for photofading caused by imaging, the location of reference region should be as far apart as possible from the bleached region. Moreover, a background region (F_{bg}) locates outside the nucleus to control for background fluorescence was also measured.

The relative fluorescence intensity in the bleached region at each time point F(t)ₙₒᵣₘ was calculated from equation 1, where F(i) is the average intensity of regions in the five pre-bleach scans.

$${F ( t )}_{norm} = \frac{{F ( t )}_{bl} - {F ( t )}_{bg}}{{F ( t )}_{ref} - {F ( t )}_{bg}} \times \frac{{F ( i )}_{ref} - {F ( i )}_{bg}}{{F ( i )}_{bl} - {F ( i )}_{bg}} \qquad equation 1$$

One-phase association curves were fitted to the normalized data using GraphPad Prism 8.2.1, which has the formula shown in equation 2.

y=y₀+(plateau−y₀)×(1−e^{-k*x}) equation 2:

Half time of fluorescence recovery (t_{1/2}) was returned by the curve fitting.

### 6.4.9 Isothermal Titration Calorimetry Measurements

Experiments were conducted at 25° C. on a MicroCal PEAQ-ITC titration calorimeter (MicroCal). The buffer for the titration is (150 mM NaCl, 50 mM HEPES, 1 mM TCEP, pH 7.5). Proteins were dialyzed to the titration buffer for overnight before the experiment. The reaction cell contained 200 μL of 30 μM YEATS domain proteins, which was titrated with 300 μM V3 or V3C proteins (10-fold higher than the YEATS domain protein concentration used). The titration contained 20 injections, first 0.5 μL, and all subsequent injections of 2 μL. The binding isotherm was fit with MicroCal PEAQ-ITC analysis software, using a single set of independent sites to determine the thermodynamic dissociation constants and stoichiometry.

### 6.5.9 Immunoblotting

Whole cell lysate was extracted by SDS lysis buffer (20 mM Tris-HCl pH 8.0, 150 mM NaCl, 1.5 mM MgCl₂, 1% SDS, add Benzonase nuclease (Thermo) before use). Protein concentrations were measured by BCA assay (Thermo) and adjusted to the same concentration. Proteins were then resolved by SDS-PAGE and transferred onto a PVDF membrane. The membrane was blocked by 5% non-fat milk in Tris-buffered saline (TBS) with 0.1% Tween-20 (TBST) for 1 hour at room temperature and was incubated with the primary antibody in antibody dilution buffer (5% bovine serum albumin in TBST) with gentle shaking for overnight at 4° C. The membrane was then washed with TBST three times and incubated with HRP conjugated secondary antibodies (Santa Cruz Biotechnologies) in 5% non-fat milk in TBST for 1 hour at room temperature. After washing by TBST three times, the proteins were visualized with SuperSignal™ west dura extended duration substrate using MyECL Imager system (Thermo Fisher Scientific).

### 6.5.10 Immunofluorescence

HEK 293 T cells were transfected with plasmids encoding the genetically encoded inhibitor. 24 h after transfection, cells were fixed with 4% polyformaldehyde in PBS for 15 min at room temperature, permeabilized with 0.5% Triton X-100 in PBS for 5 min, and blocked for 30 min at room temperature using blocking buffer (5% bovine serum albumin in PBS containing 0.1% Triton X-100). Cells were incubated with the primary antibody in blocking buffer overnight at 4° C. and washed three times with PBST (0.1% Tween-20 in PBS). Cells were then incubated with secondary antibody in blocking buffer at room temperature for 1 h, followed by counterstaining with DAPI (Thermo Fisher Scientific). After being washed three times by PBST, the cells were then mounted using ProLong antifade reagent (Thermo Fisher Scientific) and analyzed by Zeiss LSM 710 laser scanning confocal microscope.

### 6.5.11 Chromatin Immunoprecipitation (ChIP)

HEK 293T cells were seeded in 10 cm dish, and 1 mM Kfu was added to corresponding cells 1 h prior transfection. Cells were then transfected with pNEU-KfuRS and pCMV-V3-NLS-PylTM15 or pCMV-V3C-NLS-PylTM15 plasmid. 48 h after transfection, cells were cross-linked by addition of 1/10 volume of 10× crosslinking solution (11% formaldehyde, 50 mM HEPES pH 7.3, 100 mM NaCl, 1 mM EDTA pH 8.0, 0.5 mM EGTA pH 8.0) for 10 min and quenched by 0.125 M glycine for 5 min at room temperature, cells were then washed with PBS for three times, frozen in liquid nitrogen and stored in −80° C. Cells were thawed and lysed by cold lysis buffer 1 (50 mM HEPES pH 7.3, 140 mM NaCl, 1 mM EDTA, 10% glycerol, 0.5% NP-40, and 0.25% Triton X-100, Roche protease inhibitor cocktail) and rotated for 10 minutes at 4° C. Lysis buffer 1 was removed and pellets were resuspended in cold lysis buffer 2 (10 mM Tris-HCl pH 8.0, 200 mM NaCl, 1 mM EDTA pH 8.0 and 0.5 mM EGTA pH 8.0, Roche protease inhibitor cocktail) and rotated for 10 minutes at 4° C. Lysis buffer 2 was removed and pellets were resuspended in cold sonication buffer (50 mM HEPES pH 7.3, 140 mM NaCl, 1 mM EDTA, 1 mM EGTA, 1% Triton X-100, 0.1% Na-deoxycholate, 0.1% SDS, Roche protease inhibitor cocktail). The chromatin was sonicated to a DNA fragment ranging from 1000 bp to 1500 bp using a VCX750 ultrasonic processors (Sonics & Materials) with microtip (35% amplitude; 1 s on, 4 s off for 1.2 min). The chromatin was then cleared by centrifugation (20,000 g, 10 min). To quantify the chromatin, an aliquot of cleared chromatin was reverse cross-linked by heating at 95° C. for 20 min and then treated with 0.2 mg/mL RNase A (Thermo Fisher Scientific) for 20 min at 37° C. DNA was recovered using QIAquick PCR Purification Kit (QIAGEN) and quantified by NanoDrop spectrophotometers (Thermo Fisher Scientific). The cleared chromatin was diluted to 25 ng/μL by sonication buffer and 10 μg chromatin was used for one immunoprecipitation reaction (5 μg chromatin was saved as input sample).

Protein A/G magnetic beads (Thermo Scientific, ChIP grade) were washed with blocking buffer (5 mg/mL BSA in PBS pH 7.4) three times. The primary antibody was incubated with protein A/G magnetic beads in blocking buffer for overnight at 4° C. Antibody-beads mixture was washed with blocking buffer three times and then added to diluted chromatin and incubated at 4° C. for overnight. The bound chromatin was sequentially washed with 0.5 mL cold sonication buffer for twice, with 0.5 mL cold sonication buffer supplemented with 500 mM NaCl for once, with cold LiCI wash buffer (20 mM Tris pH 8.0, 1 mM EDTA, 250 mM LiCl, 0.5% NP-40, 0.5% Na-deoxycholate) for once, and with TE supplemented with 50 mM NaCl for once. Finally, beads were resuspended in 100 μL elution buffer (50 mM Tris-HCl pH 8, 10 mM EDTA, and 1% SDS), and chromatin was eluted by incubating at 65° C. with vortexing for 15 min. The eluate was clarified by centrifugation, and the clear supernatant, together with the input sample, was reverse cross-linked at 65° C. for overnight. After treated with 0.2 mg/mL RNase A (Thermo Fisher Scientific) for 2 h at 37° C. and 0.2 mg/mL protease K (Thermo Fisher Scientific) for 30 min at 55° C., DNA was recovered using QIAquick PCR purification kit (QIAGEN) and subjected to quantitative PCR analyses.

### 6.5.12 Quantitative PCR Analysis (qPCR)

DNA recovered from ChIP was quantified by qPCR, which was performed using Power SYBR Green PCR Master Mix (Invitrogen) on an ABI StepOnePlus system following the manual's instructions. The relative DNA amount was analyzed using the ΔΔCt method and normalized to the input sample. Primers used for ChIP-qPCR were listed in Appendix Table 1.

### 6.5.13 LacO-lacR Tethering Experiment

U2OS-LacO was purchased from kerafast company, and cultured in DMEM medium supplemented with 10% FBS and 100 μg/mL hygromycin. One day prior transfection, U2OS-LacO cells were seeded in 3.5 cm confocal dish in antibiotic-free medium. One hour prior transfection, U2OS-LacO cells were changed to antibiotic-free medium plus 2 mM Kfu. The transfection was performed with Effectene (Qiagen) following manufacture's instruction.

Plasmids encoding AF9-mCherry, KfuRS and V3-sfGFP-LacR/V3C-sfGFP-LacR were transfected at 1:1:1 ratio (250 ng each/3.5 cm dish). Living cell imaging was performed with LSM710 NLO confocal microscope 24 hours after transfection.

### 6.5.14 Synthesis of 2-Furancarbonyl Lysine

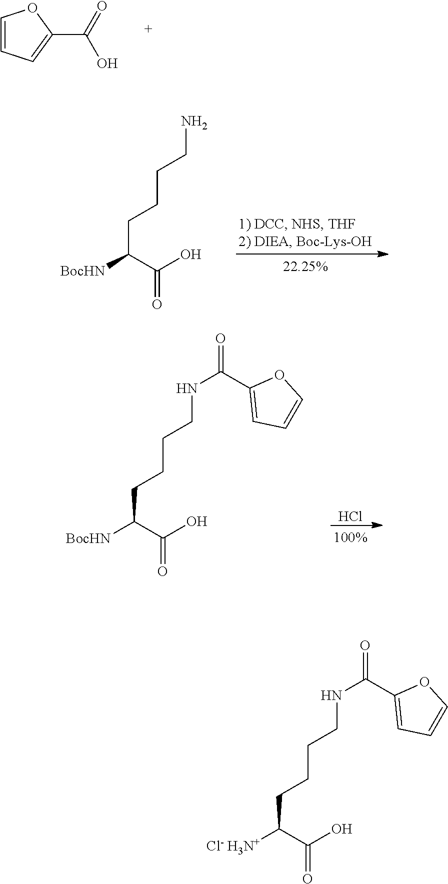

Furan acid (3 eq.) and N-Hydroxysuccinimide (2.9 eq.) was dissolved in anhydrous THF and cooled to 0° C. N,N′-Dicyclohexylcarbodiimide (2.9 eq.) was added to the mixture in two portions. The mixture was returned to room temperature and stirred for 24 hours. The resulting slurry was filtered through a pad of celite. Diisopropylethylamine (5.8eq.) was added to the filtrate, followed by the addition of Fmoc-lys-OH·HCl (2.5eq.). The mixture was stirred at room temperature for another 24 hours. Solvent was removed, the resulting residue was taken up by ethyl acetate and washed with 0.1M HCl, brine and dried with anhydrous Na₂SO₄. After removal of solvent, the residue was purified by flash column chromatography.

The resulting compound from last step was dissolved in 4 M HCl/THF (1:1). The mixture was stirred at room temperature for 3 h. Then solvent was removed under reduced pressure, the resulting yellow solid obtained was the product (2-furancarbonyl lysine).

Yellow solid, 100%. 1H NMR (500 MHz, MeOD) δ 7.66 (d, J=1.2 Hz, 1H), 7.10 (dd, J=2.5, 0.8 Hz, 1H), 6.58 (dd, J=3.3, 1.6 Hz, 1H), 3.98 (t, J=5.8 Hz, 1H), 3.38 (t, J=7.0 Hz, 2H), 2.06-1.87 (m, 2H), 1.73-1.62 (m, 2H), 1.61-1.42 (m, 2H). 13C NMR (126 MHz, MeOD) δ 171.79, 160.98, 148.96, 146.33, 115.16, 112.95, 53.84, 39.65, 31.15, 30.10, 23.34. HRMS (ESI) calculated m/z for [M+H]⁺: 241.1183. found 241.1183.

### 6.6. Appendix

TABLE 2
List of DNA oligo used

| Oligo | Sequence (5′-3′) |
|---|---|
| TGF111 | GCTAGATCTGGGAACCTGATCATGTAGATCGAATGGA |
| | CTCTAAATCCGTTCAGCC (SEQ ID NO: 4) |
| TGF112 | GATACTAGTTGGCGGAAACCCCGGGAATCTAACCCG |
| | GCTGAACGGATTTAGAGTC (SEQ ID NO: 5) |
| TGF113 | CTAGATCTATGACTAGTATCCTTAGCGAAAGCTAA |
| | (SEQ ID NO: 6) |
| TGF114 | ATACTAGTCATAGATCTAGCGTTACAAGTATTACA |
| | (SEQ ID NO: 7) |
| TGF117 | TCCCATGGTTATGACAACTTGACGGCTACATCATTCA C |
| | (SEQ ID NO: 8) |
| TGF118 | CGCATATGTTAATTCCTCCTGTTAGCCCAAAAAACGG G |
| | (SEQ ID NO: 9) |
| TGF119 | CACTGCAGGAAATGAGGCCGCTCATGGC (SEQ ID NO: 10) |
| TGF120 | CGGAATTCGTAACGCCAGGGTTTTCCCAG (SEQ ID NO: 11) |
| TGF121 | ACCATGGTGGCTTACTATGTTGGCACTGATGAGGGTG |
| | (SEQ ID NO: 12) |
| TGF122 | GACTGCAGATGCGCCGCGTGCGGCTG |
| | (SEQ ID NO: 13) |
| TGF141 | AGTCTAGATGCGTTTCTACAAACTCTTTTGTTTATTTT TC |
| | (SEQ ID NO: 14) |
| TGF142 | AGCTGCAGAGTGGAACGAAAACTCACGTTAAGGG |
| | (SEQ ID NO: 15) |
| TGF156 | GGGCCTGGTAAAATCCTATCGAGTTTTCGMNNATAGT |
| | TCSCAAGAGTCG |
| | (SEQ ID NO: 16) |
| TGF157 | CCCGAGTACATCCCGAACCCATCTGMNNGAAGTTCA |
| | CCATAG |
| | (SEQ ID NO: 17) |
| TGF158 | GCGTTCAAGACCAAAACCTGCACCTATMNNTGGTTTG |
| | TCAATACCCC |
| | (SEQ ID NO: 18) |
| TGF159 | ATAGGTGCAGGTTTTGGTCTTGAACGC |
| | (SEQ ID NO: 19) |
| TGF169-1 | AAAGTTCAGTATCATTATTAATACCCATTCTCTCCACG |
| | TATTC (SEQ ID NO: 20) |
| TGF169-1 | AAAGTTCAGTATCATTATTAATACCCCATCTCTCCAC |
| | GTATTC (SEQ ID NO: 21) |
| TGF169-1 | AAAGTTCAGTATCATTATTAATACCAHNTCTCTCCAC |
| | GTATTC (SEQ ID NO: 22) |
| TGF169-1 | AAAGTTCAGTATCATTATTAATACCTKBTCTCTCCACG |
| | TATTC (SEQ ID NO: 23) |
| TGF170 | GGTATTAATAATGATACTGAACTTTC (SEQ ID NO: 24) |
| TGF171-1 | GTAAAATCCTATCGAGTTTTCGAGCATAGTTCATAAG |
| | AGTCGGGGCAAG (SEQ ID NO: 25) |
| TGF171-2 | GTAAAATCCTATCGAGTTTTCGAGCATAGTTCCAAAG |
| | AGTCGGGGCAAG (SEQ ID NO: 26) |
| TGF171-3 | GTAAAATCCTATCGAGTTTTCGAGCATAGTTAHNAAG |
| | AGTCGGGGCAAG (SEQ ID NO: 27) |
| TGF171-4 | GTAAAATCCTATCGAGTTTTCGAGCATAGTTTKBAAG |
| | AGTCGGGGCAAG (SEQ ID NO: 28) |
| TGF172-1 | CGTGCATTATATCAAGAGTATCCCCCATGACCATACA |
| | GGAATC (SEQ ID NO: 29) |
| TGF172-2 | CGTGCATTATATCAAGAGTATCCCCCCAGACCATACA |
| | GGAATC (SEQ ID NO: 30) |
| TGF172-3 | CGTGCATTATATCAAGAGTATCCCCAHNGACCATACA |
| | GGAATC (SEQ ID NO: 31) |
| TGF172-4 | CGTGCATTATATCAAGAGTATCCCCTKBGACCATACA |
| | GGAATC (SEQ ID NO: 32) |
| TGF173 | GGGGATACTCTTGATATAATGCACG (SEQ ID NO: 33) |
| TGF174-1 | GTTTGTCAATACCCCATTCTCTATCCATAGAAACTGG |
| | CCCGAC (SEQ ID NO: 34) |
| TGF174-1 | GTTTGTCAATACCCCATTCTCTATCCCAAGAAACTGG |
| | CCCGAC (SEQ ID NO: 35) |
| TGF174-1 | GTTTGTCAATACCCCATTCTCTATCAHNAGAAACTGG |
| | CCCGAC (SEQ ID NO: 36) |
| TGF174-1 | GTTTGTCAATACCCCATTCTCTATCTKBAGAAACTGG |
| | CCCGAC (SEQ ID NO: 37) |
| TGF175 | GATAGAGAATGGGGTATTGACAAAC (SEQ ID NO: 38) |
| TGF179 | GTATCTGCGCAGTAAGATGCGCCCCGCATTGGGAACC |
| | TGATCATGTAGATC (SEQ ID NO: 39) |
| TGF180 | AGCCTGCTCGTTGAGCAGGCTTTTCGAATTTGGCGGA |
| | AACCCCGGGAATC (SEQ ID NO: 40) |
| TGF181 | GCATTTTGCTATTAAGGGATTGACGAGGGCGTATCTG |
| | CGCAGTAAGATGC (SEQ ID NO: 41) |
| TGF182 | GTCTCGAGCATGCAAAAAAGCCTGCTCGTTGAGCAGG C |
| | (SEQ ID NO: 42) |
| TGF183 | GGCCTGCTGACTTTCTCGCCGATCAAAAGGCATTTTG |
| | CTATTAAGGG (SEQ ID NO: 43) |
| TGF184 | CAGTGCACGGCTAACTAAGCGGCCTGCTGACTTTCTC GCC |
| | (SEQ ID NO: 44) |
| TGF185 | CGACTAGTATGGATAAAAAACCATTAGATG (SEQ ID NO: 45) |
| TGF193 | GCACTAGTAATTCCTCCTGTTAGCCCAAAAAAAC |
| | (SEQ ID NO: 46) |
| TGF194 | AAGTCGACCATCATCATCATCATCATTGAG (SEQ ID NO: 47) |
| TGF255 | GCTCAAAAGCAGGTAACTATATAACAAGACTAAGGC |
| | AAACCGGATCCCCGGGTTAATTAA (SEQ ID NO: 48) |
| TGF256 | CCAGACCTGATGAAATTCTTGCGCATAACGTCGCCAT |
| | CTGGAATTCGAGCTCGTTTAAAC (SEQ ID NO: 49) |
| TGF257 | GTTACTGAAGAGTACGTGAGCG (SEQ ID NO: 50) |
| TGF258 | ATGAAAGAAGAACCTCAGTGGC (SEQ ID NO: 51) |
| TGF281 | CTCAAGCTTATGGTATATCGTAACAGGTCAAAGAGCG |
| | (SEQ ID NO: 52) |
| TGF283 | TATTCTAGAGACTACAGTATAAAAATAAATAAAAATG |
| | GGCATCACAGAAAC (SEQ ID NO: 53) |
| TGF293 | TCTCTAGAGAAAACCTGTATTTTCAGTCCGGCGCCGG |
| | CACCCCGGTGACCGCCCCGCTGG (SEQ ID NO: 54) |
| TGF294 | GTGACCGCCCCGCTGGCGGGCACTATCTGGAAGGTGC |
| | TGGCCAGCGAAGGCCAGACGGTG (SEQ ID NO: 55) |
| TGF295 | GCGAAGGCCAGACGGTGGCCGCAGGCGAGGTGCTGC |
| | TGATTCTGGAAGCCATGAAGATGG (SEQ ID NO: 56) |
| TGF296 | CTGGAAGCCATGAAGATGGAAACCGAAATCCGCGCC |
| | GCGCAGGCCGGGACCGTGCGCGG (SEQ ID NO: 57) |
| TGF297 | GGCCGGGACCGTGCGCGGTATCGCGGTGAAAGCCGG |
| | CGACGCGGTGGCGGTCGGCGACAC (SEQ ID NO: 58) |
| TGF298 | GGAGATCTTCACGCCAGGGTCATCAGGGTGTCGCCGA |
| | CCGCCAC (SEQ ID NO: 59) |
| TGF317 | GGTTAATTCCTCCTGTTAGCC (SEQ ID NO: 60) |
| TGF318 | GTTAGCAAAGGTGAAGAACTG (SEQ ID NO: 61) |
| TGF319 | GGCTAACAGGAGGAATTAACCATGAAAAGGCCGGCG |
| | GCCACGAAAAAGGCCGGCCAGGCA (SEQ ID NO: 62) |
| TGF320 | GAAAAAGGCCGGCCAGGCAAAAAAGAAAAAGGGTG |
| | GCGGCGGTAGCAAGCAGACCGCCCG (SEQ ID NO: 63) |
| TGF321 | CAGTTCTTCACCTTTGCTAACCCCTCCGGTGGACTAAC |
| | GGGCGGTCTGCTTGC (SEQ ID NO: 64) |
| TGF322 | CAGTTCTTCACCTTTGCTAACCCCTCCGGTGGATTTAC |
| | GGGCGGTCTGCTTGC (SEQ ID NO: 65) |
| TGF339 | GGCGGTAGCGTTAGCAAAGGTGAAGAACTGTTTACC |
| | GGCGTTGTG (SEQ ID NO: 66) |
| TGF340 | TTTGCTAACGCTACCGCCGCCACCCTTTTTCTTTTTTG |
| | CCTGGC (SEQ ID NO: 67) |
| TGF341 | TTAACCATGAAGCAGACCGCCCGTTAGTCCACCGGAG |
| | GGGTTAGCAAAGGTGAAGAACTG (SEQ ID NO: 68) |
| TGF342 | TCTGCTTCATGGTTAATTCCTCCTGTTAGCCC |
| | (SEQ ID NO: 69) |
| TGF343 | TTAACCATGAAGCAGACCGCCCGTAAATCCACCGGA |
| | GGGGTTAGCAAAGGTGAAGAACTG (SEQ ID NO: 70) |
| TGF344 | TTAACCATGAAGCAGACCGCCCGTTAGAGTACCGGA |
| | GGGGTTAGCAAAGGTGAAGAACTG (SEQ ID NO: 71) |
| TGF345 | TTAACCATGAAGCAGACCGCCCGTTAGAGCACCGGA |
| | GGGGTTAGCAAAGGTGAAGAACTG (SEQ ID NO: 72) |
| TGF329 | AGAATTTACTATGGTGAACTTCTTTCAGATGGGTTCG |
| | GGATGTACTC (SEQ ID NO: 73) |
| TGF346 | CATGCTGGCCCCCACCCTGTATAACTACGCGCGGAAA |
| | CTGGACCGGATCCTG (SEQ ID NO: 74) |
| TGF347 | GCAGCCGCTGCCCATCTGAAAGAAGTTGACCATTGTA |
| | AACTCTTCCAGG (SEQ ID NO: 75) |
| TGF348 | CATGCTGGCCCCCAACCTGTATAACTACGCGCGGAAA |
| | CTGGATCGCGCTC (SEQ ID NO: 76) |
| TGF349 | GCAGCCGCTGCCCATCTGAAAAAAGTTCAGCATTGTA |
| | AACTCTTCCAG (SEQ ID NO: 77) |
| TGF350 | GGTGGCGCTAGCCAGCTTGG (SEQ ID NO: 78) |
| TGF351 | GCGGCCGCTTTAAACCCGC (SEQ ID NO: 79) |
| TGF352 | CCAAGCTGGCTAGCGCCACCATGAAGCAGACCGCCC |
| | GTTAG (SEQ ID NO: 80) |
| TGF353 | GCGGGTTTAAAGCGGCCGCTTACTTCTTCTTCTTTGCC |
| | TGGCCG (SEQ ID NO: 81) |
| TGF354 | GGTGGCAAGCTTCCGTGCAG (SEQ ID NO: 82) |
| TGF355 | ACCCGCTGATCAGCCTCGAC (SEQ ID NO: 83) |
| TGF356 | CTGCACGGAAGCTTGCCACCATGAAGCAGACCGCCC |
| | GTTAG (SEQ ID NO: 84) |
| TGF357 | GTCGAGGCTGATCAGCGGGTTTACTTCTTCTTCTTTGC |
| | CTGGCCG (SEQ ID NO: 85) |
| TGF387 | GGATTTAGAGTCCGTTCGGTCTCCCTGACCAGGTTTC |
| | CGGTGTTTCGTC (SEQ ID NO: 86) |
| TGF388 | GAACGGACTCTAAATCCGTTCAGCCGGGTTCGATTCC |
| | CGGGGTTTCCGC (SEQ ID NO: 87) |
| TGF379 | CCCGTAAAAGCACCGGAGGGGTGTCCAAGGG |
| | (SEQ ID NO: 88) |
| TGF380 | GGTGCTTTTACGGGCGGTCTGCTTCATGGTGG |
| | (SEQ ID NO: 89) |
| TGF389 | CCGCTTGAGAGACTTACTCTTTAAACCCGCTGATCAG |
| | CCTCG (SEQ ID NO: 90) |
| TGF390 | AGTAAGTCTCTCAAGCGGTGGTAGTCCGCCTCCGCCA |
| | TGGTGATG (SEQ ID NO: 91) |
| TGF396 | GCAGCTAAACCCGCTGATCAGCCTCGACTGTGCCTTC |
| | TAGTTG (SEQ ID NO: 92) |
| TGF397 | CAGCGGGTTTAGCTGCCCTTGTACAGCTCGTCCATGC CG |
| | (SEQ ID NO: 93) |
| TGF431 | CCTTGCTCACCCCTCCGGTGCTCTAACGG (SEQ ID NO: 94) |
| TGF432 | GCTGTACAAGCACCATCACCATCACCATGGC |
| | (SEQ ID NO: 95) |
| TGF433 | CCTTGCTCACCCCTCCGGTGCTTTTACGG (SEQ ID NO: 96) |
| TGF434 | CACCGGAGGGGTGAGCAAGGGCGAGGAGG |
| | (SEQ ID NO: 97) |
| TGF435 | GGTGATGGTGCTTGTACAGCTCGTCCATGCC |
| | (SEQ ID NO: 98) |
| TGF375 | GGTGGCGCTAGCCAGCTTG (SEQ ID NO: 99) |
| TGF376 | ATGGTGAGCAAGGGCGAGG (SEQ ID NO: 100) |
| TGF425 | CGAAAGCGCTCCTAGGCCAAAAAGAGTGTGCAAAG |
| | (SEQ ID NO: 101) |
| TGF426 | CTAGGAGCGCTTTCGTGCAAGTGGAAGACGACTTTC |
| | (SEQ ID NO: 102) |
| TGF427 | TGGAACATCCGTGTCCAAGGGCGAGGAACTG |
| | (SEQ ID NO: 103) |
| TGF428 | GTTTTTGTTCGCTGCCCTTGTACAGCTCGTC |
| | (SEQ ID NO: 104) |
| TGF429 | CCTTGGACACGGATGTTCCAGATGTTTCCAGGTAACT C |
| | (SEQ ID NO: 105) |
| TGF430 | CAAGGGCAGCGAACAAAAACTTATTTCTGAAGAAGA |
| | TCTGTAACTCGAG (SEQ ID NO: 106) |
| TGF105 | GAGGAATCCCATATGGATAAAAAACCATTAGATG |
| | (SEQ ID NO: 107) |
| TGF106 | GGGCCTGGTAAAATCCTATCGAGTTTTCGMNNATAGT |
| | TMNNAAGAGTCG (SEQ ID NO: 108) |
| TGF107 | CGAAAACTCGATAGGATTTTACCAGGCCC |
| | (SEQ ID NO: 109) |
| TGF157 | CCCGAGTACATCCCGAACCCATCTGMNNGAAGTTCA |
| | CCATAG (SEQ ID NO: 110) |
| TGF109 | CAGATGGGTTCGGGATGTACTCGGG (SEQ ID NO: 111) |
| TGF110 | GAAACTGCAGTTATAGATTGGTTGAAATCCC |
| | (SEQ ID NO: 112) |
| MYC-ChIP-F | AAGGGAGGCGAGGATGTGT (SEQ ID NO: 113) |
| MYC-ChIP-F | TTCGCCCTGGTTTTTCCAA (SEQ ID NO: 114) |
| PABPC1-ChIP-F | CAGCGGCAGTGGATCGA (SEQ ID NO: 115) |
| PABPC1-ChIP-F | CAACCGGAATTGAAAACTACTCAA (SEQ ID NO: 116) |

### REFERENCES

1. Kouzarides, T. Chromatin modifications and their function. Cell 128, 693-705 (2007).
2. Kouzarides, T. SnapShot: Histone-modifying enzymes. Cell 131, 822 (2007).
3. Taverna, S. D., Li, H., Ruthenburg, A. J., Allis, C. D. & Patel, D. J. How chromatin-binding modules interpret histone modifications: lessons from professional pocket pickers. Nat Struct Mol Biol 14, 1025-1040 (2007).
4. Musselman, C. A., Lalonde, M. E., Cote, J. & Kutateladze, T. G. Perceiving the epigenetic landscape through histone readers. Nat Struct Mol Biol 19, 1218-1227 (2012).
5. Patel, D. J. & Wang, Z. Readout of epigenetic modifications. Annual review of biochemistry 82, 81-118 (2013).
6. Chi, P., Allis, C. D. & Wang, G. G. Covalent histone modifications—miswritten, misinterpreted and mis-erased in human cancers. Nature reviews. Cancer 10, 457-469 (2010).
7. Bhaumik, S. R., Smith, E. & Shilatifard, A. Covalent modifications of histones during development and disease pathogenesis. Nat Struct Mol Biol 14, 1008-1016 (2007).
8. Audia, J. E. & Campbell, R. M. Histone Modifications and Cancer. Cold Spring Harbor perspectives in biology 8, a019521 (2016).
9. Kim, H. J. & Bae, S. C. Histone deacetylase inhibitors: molecular mechanisms of action and clinical trials as anti-cancer drugs. American journal of translational research 3, 166-179 (2011).
10. Filippakopoulos, P. & Knapp, S. Targeting bromodomains: epigenetic readers of lysine acetylation. Nature reviews. Drug discovery 13, 337-356 (2014).
11. Li, X. et al. Chemical Proteomic Profiling of Bromodomains Enables the Wide-Spectrum Evaluation of Bromodomain Inhibitors in Living Cells. Journal of the American Chemical Society 141, 11497-11505 (2019).
12. Li, Y. et al. AF9 YEATS domain links histone acetylation to DOT1L-mediated H3K79 methylation. Cell 159, 558-571 (2014).
13. Zhao, D., Li, Y., Xiong, X., Chen, Z. & Li, H. YEATS Domain-A Histone Acylation Reader in Health and Disease. J Mol Biol 429, 1994-2002 (2017).
14. Schulze, J. M., Wang, A. Y. & Kobor, M. S. YEATS domain proteins: a diverse family with many links to chromatin modification and transcription. Biochemistry and cell biology=Biochimie et biologie cellulaire 87, 65-75 (2009).
15. Collins, E. C. et al. Mouse Af9 is a controller of embryo patterning, like Mll, whose human homologue fuses with Af9 after chromosomal translocation in leukemia. Mol Cell Biol 22, 7313-7324 (2002).
16. Calvanese, V. et al. MLLT3 governs human haematopoietic stem-cell self-renewal and engraftment. Nature (2019).
17. Li, Y. et al. Molecular Coupling of Histone Crotonylation and Active Transcription by AF9 YEATS Domain. Mol Cell 62, 181-193 (2016).
18. Zhang, Q. et al. Structural Insights into Histone Crotonyl-Lysine Recognition by the AF9 YEATS Domain. Structure (London, England: 1993) 24, 1606-1612 (2016).
19. Erb, M. A. et al. Transcription control by the ENL YEATS domain in acute leukaemia. Nature 543, 270-274 (2017).
20. Wan, L. et al. ENL links histone acetylation to oncogenic gene expression in acute myeloid leukaemia. Nature 543, 265-269 (2017).
21. Mi, W. et al. YEATS2 links histone acetylation to tumorigenesis of non-small cell lung cancer. Nature communications 8, 1088 (2017).
22. Hsu, C. C. et al. Recognition of histone acetylation by the GAS41 YEATS domain promotes H2A.Z deposition in non-small cell lung cancer. Genes & development 32, 58-69 (2018).
23. Hsu, C. C. et al. Gas41 links histone acetylation to H2A.Z deposition and maintenance of embryonic stem cell identity. Cell discovery 4, 28 (2018).
24. Li, X. et al. Structure-guided development of YEATS domain inhibitors by targeting pi-pi-pi stacking. Nature chemical biology 14, 1140-1149 (2018).
25. Chin, J. W. Expanding and reprogramming the genetic code of cells and animals. Annual review of biochemistry 83, 379-408 (2014).
26. Chin, J. W. Expanding and reprogramming the genetic code. Nature 550, 53-60 (2017).
27. Wan, W., Tharp, J. M. & Liu, W. R. Pyrrolysyl-tRNA synthetase: an ordinary enzyme but an outstanding genetic code expansion tool. Biochimica et biophysica acta 1844, 1059-1070 (2014).
28. Dumas, A., Lercher, L., Spicer, C. D. & Davis, B. G. Designing logical codon reassignment
   Expanding the chemistry in biology. Chem Sci 6, 50-69 (2015).
29. Crnkovic, A., Suzuki, T., Soll, D. & Reynolds, N. M. Pyrrolysyl-tRNA synthetase, an aminoacyl-tRNA synthetase for genetic code expansion. Croatica chemica acta. Arhiv za kemiju 89, 163-174 (2016).
30. Wang, L., Brock, A., Herberich, B. & Schultz, P. G. Expanding the genetic code of Escherichia coli. Science 292, 498-500 (2001).
31. Wang, L., Xie, J. & Schultz, P. G. Expanding the genetic code. Annu Rev Bioph Biom 35, 225-249 (2006).
32. Cooley, R. B. et al. Structural basis of improved second-generation 3-nitro-tyrosine tRNA synthetases. Biochemistry 53, 1916-1924 (2014).
33. Kim, C. H., Kang, M., Kim, H. J., Chatterjee, A. & Schultz, P. G. Site-specific incorporation of epsilon-N-crotonyllysine into histones. Angewandte Chemie 51, 7246-7249 (2012).
34. Wang, Y. S., Fang, X. Q., Wallace, A. L., Wu, B. & Liu, W. S. R. A Rationally Designed Pyrrolysyl-tRNA Synthetase Mutant with a Broad Substrate Spectrum. Journal of the American Chemical Society 134, 2950-2953 (2012).
35. Guo, L. T. et al. Polyspecific pyrrolysyl-tRNA synthetases from directed evolution. Proc. Natl. Acad. Sci. U.S.A. 111, 16724-16729 (2014).
36. Ryu, Y. & Schultz, P. G. Efficient incorporation of unnatural amino acids into proteins in Escherichia coli. Nature methods 3, 263-265 (2006).
37. Young, T. S., Ahmad, I., Yin, J. A. & Schultz, P. G. An enhanced system for unnatural amino acid mutagenesis in E. coli. J Mol Biol 395, 361-374 (2010).
38. Pott, M., Schmidt, M. J. & Summerer, D. Evolved sequence contexts for highly efficient amber suppression with noncanonical amino acids. ACS chemical biology 9, 2815-2822 (2014).
39. Xu, H. et al. Re-exploration of the Codon Context Effect on Amber Codon-Guided Incorporation of Noncanonical Amino Acids in Escherichia coli by the Blue-White Screening Assay. Chembiochem 17, 1250-1256 (2016).
40. Chemla, Y., Ozer, E., Algov, I. & Alfonta, L. Context effects of genetic code expansion by stop codon suppression. Current opinion in chemical biology 46, 146-155 (2018).
41. Bossi, L. & Roth, J. R. The influence of codon context on genetic code translation. Nature 286, 123-127 (1980).
42. Bossi, L. Context effects: translation of UAG codon by suppressor tRNA is affected by the sequence following UAG in the message. J Mol Biol 164, 73-87 (1983).
43. Miller, J. H. & Albertini, A. M. Effects of surrounding sequence on the suppression of nonsense codons. J Mol Biol 164, 59-71 (1983).
44. Schwer, B., Bunkenborg, J., Verdin, R. O., Andersen, J. S. & Verdin, E. Reversible lysine acetylation controls the activity of the mitochondrial enzyme acetyl-CoA synthetase 2. Proc Natl Acad Sci USA 103, 10224-10229 (2006).
45. Neumann, H., Peak-Chew, S. Y. & Chin, J. W. Genetically encoding N-epsilon-acetyllysine in recombinant proteins. Nature chemical biology 4, 232-234 (2008).
46. Serfling, R. et al. Designer tRNAs for efficient incorporation of non-canonical amino acids by the pyrrolysine system in mammalian cells. Nucleic acids research 46, 1-10 (2018).
47. Schmied, W. H., Elsasser, S. J., Uttamapinant, C. & Chin, J. W. Efficient multisite unnatural amino acid incorporation in mammalian cells via optimized pyrrolysyl tRNA synthetase/tRNA expression and engineered eRF1. Journal of the American Chemical Society 136, 15577-15583 (2014).
48. French, C. A. et al. BRD-NUT oncoproteins: a family of closely related nuclear proteins that block epithelial differentiation and maintain the growth of carcinoma cells. Oncogene 27, 2237-2242 (2008).
49. Filippakopoulos, P. et al. Selective inhibition of BET bromodomains. Nature 468, 1067-1073 (2010).
50. Philpott, M. et al. Assessing cellular efficacy of bromodomain inhibitors using fluorescence recovery after photobleaching. Epigenetics & chromatin 7, 14 (2014).
51. Zhang, M. S. et al. Biosynthesis and genetic encoding of phosphothreonine through parallel selection and deep sequencing. Nature methods (2017).
52. Bryson, D. I. et al. Continuous directed evolution of aminoacyl-tRNA synthetases. Nature chemical biology 13, 1253-1260 (2017).
53. Suzuki, T. et al. Crystal structures reveal an elusive functional domain of pyrrolysyl-tRNA synthetase. Nature chemical biology 13, 1261-1266 (2017).
54. Liu, C. C. & Schultz, P. G. Adding new chemistries to the genetic code. Annual review of biochemistry 79, 413-444 (2010).
55. Neumann, H., Wang, K., Davis, L., Garcia-Alai, M. & Chin, J. W. Encoding multiple unnatural amino acids via evolution of a quadruplet-decoding ribosome. Nature 464, 441-444 (2010).
56. Guo, J. T., Melancon, C. E., Lee, H. S., Groff, D. & Schultz, P. G. Evolution of Amber Suppressor tRNAs for Efficient Bacterial Production of Proteins Containing Nonnatural Amino Acids. Angewandte Chemie-International Edition 48, 9148-9151 (2009).
57. Fan, C. G., Xiong, H., Reynolds, N. M. & Soll, D. Rationally evolving tRNA(Pyl) for efficient incorporation of noncanonical amino acids. Nucleic acids research 43 (2015).
58. Park, H. S. et al. Expanding the Genetic Code of Escherichia coli with Phosphoserine.

Science 333, 1151-1154 (2011).
59. Ernst, A. et al. A strategy for modulation of enzymes in the ubiquitin system. Science 339, 590-595 (2013).
60. Gorelik, M. et al. A Structure-Based Strategy for Engineering Selective Ubiquitin Variant Inhibitors of Skp1-Cul1-F-Box Ubiquitin Ligases. Structure (London, England: 1993) 26, 1226-1236 e1223 (2018).
61. Garg, P. et al. Structural and Functional Analysis of Ubiquitin-based inhibitors that Target the Backsides of E2 Enzymes. J Mol Biol (2019).
62. Teyra, J. et al. Structural and Functional Characterization of Ubiquitin Variant Inhibitors of USP15. Structure (London, England: 1993) 27, 590-605 e595 (2019).
63. Canny, M. D. et al. Inhibition of 53BP1 favors homology-dependent DNA repair and increases CRISPR-Cas9 genome-editing efficiency. Nature biotechnology 36, 95-102 (2018).
64. Leung, I., Jarvik, N. & Sidhu, S. S. A Highly Diverse and Functional Naive Ubiquitin Variant Library for Generation of Intracellular Affinity Reagents. J Mol Biol 429, 115-127 (2017).
65. Guntas, G. et al. Engineering a genetically encoded competitive inhibitor of the KEAP1-NRF2 interaction via structure-based design and phage display. Protein engineering, design & selection: PEDS 29, 1-9 (2016).
66. Heumuller, M., Glock, C., Rangaraju, V., Biever, A. & Schuman, E. M. A genetically encodable cell-type-specific protein synthesis inhibitor. Nature methods 16, 699-702 (2019).
67. Vlieghe, P., Lisowski, V., Martinez, J. & Khrestchatisky, M. Synthetic therapeutic peptides: science and market. Drug Discov Today 15, 40-56 (2010).
68. Miersch, S. & Sidhu, S. S. Intracellular targeting with engineered proteins. F1000Research 5(2016).
69. Yanagisawa, T. et al. Multistep engineering of pyrrolysyl-tRNA synthetase to genetically encode N(epsilon)-(o-azidobenzyloxycarbonyl) lysine for site-specific protein modification. Chemistry & biology 15, 1187-1197 (2008).
70. Nguyen, D. P. et al. Genetic Encoding and Labeling of Aliphatic Azides and Alkynes in Recombinant Proteins via a Pyrrolysyl-tRNA Synthetase/tRNA(CUA) Pair and Click Chemistry. Journal of the American Chemical Society 131, 8720-+(2009).
71. Takimoto, J. K., Dellas, N., Noel, J. P. & Wang, L. Stereochemical basis for engineered pyrrolysyl-tRNA synthetase and the efficient in vivo incorporation of structurally divergent non-native amino acids. ACS chemical biology 6, 733-743 (2011).
72. Wang, Y. S. et al. The de novo engineering of pyrrolysyl-tRNA synthetase for genetic incorporation of L-phenylalanine and its derivatives. Molecular bioSystems 7, 714-717 (2011).
73. Lacey, V. K., Louie, G. V., Noel, J. P. & Wang, L. Expanding the Library and Substrate Diversity of the Pyrrolysyl-tRNA Synthetase to Incorporate Unnatural Amino Acids Containing Conjugated Rings. Chembiochem 14, 2100-2105 (2013).
74. Xiao, H. et al. Genetic Incorporation of Histidine Derivatives Using an Engineered Pyrrolysyl-tRNA Synthetase. ACS chemical biology 9, 1092-1096 (2014).
75. Elsasser, S. J., Ernst, R. J., Walker, O. S. & Chin, J. W. Genetic code expansion in stable cell lines enables encoded chromatin modification. Nature methods 13, 158-164 (2016).
76. Roy, G. et al. Development of a high yielding expression platform for the introduction of non-natural amino acids in protein sequences. mAbs 12, 1684749 (2020).
77. Beckwith, J. Fifty years fused to lac. Annu Rev Microbiol 67, 1-19 (2013).
78. Hansen, L. H., Knudsen, S. & Sorensen, S. J. The effect of the lacY gene on the induction of IPTG inducible promoters, studied in Escherichia coli and Pseudomonas fluorescens. Current microbiology 36, 341-347 (1998).
79. Straight, A. F., Belmont, A. S., Robinett, C. C. & Murray, A. W. GFP tagging of budding yeast chromosomes reveals that protein-protein interactions can mediate sister chromatid cohesion. Current biology: CB 6, 1599-1608 (1996).
80. Heun, P., Laroche, T., Shimada, K., Furrer, P. & Gasser, S. M. Chromosome dynamics in the yeast interphase nucleus. Science 294, 2181-2186 (2001).
81. Soutoglou, E. & Misteli, T. Activation of the cellular DNA damage response in the absence of DNA lesions. Science 320, 1507-1510 (2008).
82. Brocken, D. J. W., Tark-Dame, M. & Dame, R. T. dCas9: A Versatile Tool for Epigenome Editing. Current issues in molecular biology 26, 15-32 (2018).
83. Moustakim, M. et al. Discovery of an MLLT1/3 YEATS Domain Chemical Probe. Angewandte Chemie 57, 16302-16307 (2018).
84. Asiaban, J. N. et al. Cell-Based Ligand Discovery for the ENL YEATS Domain. ACS chemical biology 15, 895-903 (2020).

### Exemplary Systems and Methods are Set Out in the Following Items:

Item 1. A 2-furancarbonyl lysine having the formula:

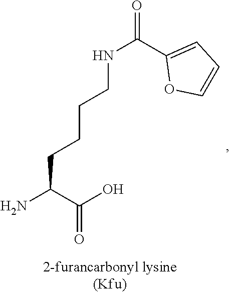 [SMILES: N[C@@H](CCCCNC(=O)c1ccco1)C(=O)O]

or salts thereof.
Item 2. A method of making a polypeptide comprising a 2-furancarbonyl lysine, said method comprises translation of a RNA encoding said polypeptide, wherein said RNA comprises an amber stop codon, and wherein said translation is carried out in the presence of a tRNA charged with 2-furancarbonyl lysine and the translation terminates at the amber stop codon.
Item 3. The method of any of the preceding items wherein the tRNA charged with 2-furancarbonyl lysine is supplied by providing a combination of tRNA capable of being charged with 2-furancarbonyl lysine, a tRNA synthetase capable of charging said tRNA with 2-furancarbonyl lysine, and in the presence of 2-furancarbonyl lysine.
Item 4. The method of any of the preceding items wherein the tRNA synthetase capable of charging said tRNA with 2-furancarbonyl lysine comprises Methanosarcina barkeri pyrrolysyl-tRNA synthetase (MbPylRS) with three mutations relative to the wild-type sequence wherein the mutations are L274A and C313F and Y349F.
Item 5. The method of any of the preceding items wherein the tRNA capable of being charged with 2-furancarbonyl lysine comprises Methanosarcina barkeri tRNA_{CUA}.
Item 6. A polypeptide comprising: (i) a histone H3-derived decapeptide; and (ii) a partner protein that fused with the H3-derived decapeptide.
Item 7. The polypeptide of any of the preceding items wherein said histone H3-derived decapeptide is from histone H3 residue 4-13 (KQTARKSTGG) (SEQ ID NO:1) comprising a 2-furancarbonyl lysine at lysine 9 position.
Item 8. The polypeptide of any of the preceding items wherein said H3-derived decapeptide is capable of binding with YEATS domains.
Item 9. The polypeptide of any of the preceding items comprises AF9.
Item 10. The polypeptide of any of the preceding items wherein the partner protein comprises a superfolder GFP (sfGFP) protein.
Item 11. A method of disrupting the interaction of YEATS domain of AF9 with chromatin comprises the step of expressing the polypeptide of any of the preceding items in mammalian cells.
Item 12. The polypeptide of any of the preceding items wherein the partner protein comprises DNA binding proteins, transcription factors and dCas9 protein.
Item 13. The polypeptide of any of the preceding items wherein the DNA binding protein comprises Lac repressor (LacR) protein.
Item 14. A method of recruiting YEATS domain protein to a specific genomic locus comprising the step of expressing the polypeptide of any of the preceding items in mammalian cells comprising a genome.
Item 15. The method of any of the preceding items wherein the YEATS domains comprise AF9.
Item 16. The method of any of the preceding items wherein the specific genomic locus comprises Lac operator (LacO) arrays which is stably integrated into the genome of the mammalian cells.
Item 17. The polypeptide of any of the preceding items wherein the DNA binding protein comprises GAL4 DNA binding domain (DBD).
Item 18. A method of recruiting YEATS-VP64 fusion protein to the GAL4 upstream activating sequence (UAS) sequence upstream of a luciferase gene by genetically express the polypeptide of any of the preceding items in mammalian cells.
Item 19. The method of any of the preceding items wherein the YEATS domains comprise AF9.
Item 20. A vector expressing the polypeptide of any of the preceding items wherein said histone H3-derived decapeptide comprises one or more 2-furancarbonyl lysine.
Item 21. A system or a cell comprising the vector of any of the preceding items.
Item 22. A method of treating a disorder comprising administering the polypeptide of any of the preceding items.
Item 23. A method of screening for a molecule that modulates YEATS domain proteins, said method comprises: (i) providing the vector of any of the preceding items; (ii) detecting binding between the polypeptide and a target molecule; and (iii) identifying a target molecule.
Item 24. The method of any of the preceding items further comprising the steps of: (i) determining whether the binding between the polypeptide and a target molecule is capable of moderating a DNA binding protein; and (ii) producing a detectable effect.
Item 25. The method of any of the preceding items wherein the method of screening is a high throughput screening.
Item 26. The method of any of the preceding items wherein the target molecule is further assessed as a candidate drug.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

What is claimed:

1. A 2-furancarbonyl lysine having the formula: 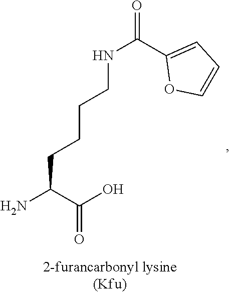 [SMILES: N[C@@H](CCCCNC(=O)c1ccco1)C(=O)O]
or salts thereof.

2. A method of making a polypeptide comprising a 2-furancarbonyl lysine, said method comprises translation of a RNA encoding said polypeptide, wherein said RNA comprises an amber stop codon, and wherein said translation is carried out in the presence of a tRNA charged with 2-furancarbonyl lysine and the translation terminates at the amber stop codon.

3. The method of claim 2 wherein the tRNA charged with 2-furancarbonyl lysine is supplied by providing a combination of tRNA capable of being charged with 2-furancarbonyl lysine, a tRNA synthetase capable of charging said tRNA with 2-furancarbonyl lysine, and in the presence of 2-furancarbonyl lysine.

4. The method of claim 3 wherein the tRNA synthetase capable of charging said tRNA with 2-furancarbonyl lysine comprises Methanosarcina barkeri pyrrolysyl-tRNA synthetase (MbPylRS) with three mutations relative to the wild-type sequence wherein the mutations are L274A and C313F and Y349F.

5. The method of claim 3 wherein the tRNA capable of being charged with 2-furancarbonyl lysine comprises Methanosarcina barkeri tRNA_{CUA}.

6. A polypeptide comprising: (i) a histone H3-derived decapeptide; and (ii) a partner protein that fused with the H3-derived decapeptide.

7. The polypeptide of claim 6 wherein said histone H3-derived decapeptide is from histone H3 residue 4-13 (KQTARKSTGG) (SEQ ID NO:1) comprising a 2-furancarbonyl lysine at lysine 9 position.

8. The polypeptide of claim 6 wherein said H3-derived decapeptide is capable of binding with YEATS domains.

9. The polypeptide of claim 8 comprises AF9.

10. The polypeptide of claim 6 wherein the partner protein comprises a superfolder GFP (sfGFP) protein.

11. A method of disrupting the interaction of YEATS domain of AF9 with chromatin comprises the step of expressing the polypeptide of claim 6 in mammalian cells.

12. The polypeptide of claim 6 wherein the partner protein comprises DNA binding proteins, transcription factors and dCas9 protein.

13. The polypeptide of claim 12 wherein the DNA binding protein comprises Lac repressor (LacR) protein.

14. A method of recruiting YEATS domain protein to a specific genomic locus comprising the step of expressing the polypeptide of claim 12 in mammalian cells comprising a genome.

15. The method of claim 14 wherein the YEATS domains comprise AF9.

16. The method of claim 14 wherein the specific genomic locus comprises Lac operator (LacO) arrays which is stably integrated into the genome of the mammalian cells.

17. The polypeptide of claim 12 wherein the DNA binding protein comprises GAL4 DNA binding domain (DBD).

18. A method of recruiting YEATS-VP64 fusion protein to the GAL4 upstream activating sequence (UAS) sequence upstream of a luciferase gene by genetically express the polypeptide of claim 17 in mammalian cells.

19. The method of claim 18 wherein the YEATS domains comprise AF9.

20. A vector expressing the polypeptide of claim 6 wherein said histone H3-derived decapeptide comprises one or more 2-furancarbonyl lysine.

21. A system or a cell comprising the vector of claim 20.

22. A method of treating a disorder comprising administering the polypeptide of claim 6.

23. A method of screening for a molecule that modulates YEATS domain proteins, said method comprises: (i) providing the vector of claim 20; (ii) detecting binding between the polypeptide and a target molecule; and (iii) identifying a target molecule.

24. The method of claim 23 further comprising the steps of: (i) determining whether the binding between the polypeptide and a target molecule is capable of moderating a DNA binding protein; and (ii) producing a detectable effect.

25. The method of claim 24 wherein the method of screening is a high throughput screening.

26. The method of claim 24 wherein the target molecule is further assessed as a candidate drug.
